(19) United States
(12) Patent Application Publication
(10) Pub. No.: US 2010/0120778 A1
(43) Pub. Date: May 13, 2010

(54) QUINOXALINE-BASED LXR MODULATORS

(75) Inventors: Baihua Hu, Audubon, PA (US); Jay Edward Wrobel, Lawrenceville, NJ (US); James Winfield Jetter, Norristown, PA (US); David John O'Neill, Collegeville, PA (US); Charles William Mann, Plymouth Meeting, PA (US); Rayomand Jal Unwalla, Eagleville, PA (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/614,167

(22) Filed: Nov. 6, 2009

Related U.S. Application Data
(60) Provisional application No. 61/112,456, filed on Nov. 7, 2008.

(51) Int. Cl. A61K 31/498; C07D 241/36; A61P 1/16; A61P 9/10; A61P 9/00; A61P 3/04; A61P 3/06; A61P 25/28; A61P 3/10; A61P 29/00; A61P 19/04

(52) U.S. Cl. 514/249; 544/353; 544/354

(57) ABSTRACT

Disclosed are quinoxaline-based modulators of Liver X receptors (LXRs) and related methods. The modulators include compounds of formula (I):

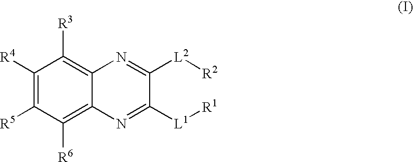

wherein:
each of L¹ and L² is, independently, a bond, —O— or —NH—;
R² is C₆-C₁₀ aryl or heteroaryl including 5-10 atoms, each of which is (i) substituted with 1 R⁹, and (ii) optionally further substituted with from 1-4 R^{e}; and
each of R⁴ and R⁵ is, independently (i) hydrogen; or (ii) halo; or (iii) C₁-C₆ alkyl or C₁-C₆ haloalkyl, each of which is optionally substituted with from 1-3 R^{a};
and R¹, R³, R⁶, R⁹, R^{a} and R^{e} are defined herein. In general, these compounds can be used for treating or preventing one or more diseases, disorders, conditions or symptoms mediated by LXRs.

### TECHNICAL FIELD

This invention relates generally to quinoxaline-based modulators of Liver X receptors (LXRs) and related methods.

### BACKGROUND

Atherosclerosis is among the leading causes of death in developed countries. Some of the independent risk factors associated with atherosclerosis include the presence of relatively high levels of serum LDL cholesterol and relatively low levels of serum HDL cholesterol in affected patients. As such, some anti-atherosclerotic therapy regimens include the administration of agents (e.g., statins) to reduce elevated serum LDL cholesterol levels.

Agents that increase patient HDL cholesterol levels can also be useful in anti-atherosclerotic therapy regimens. HDL cholesterol is believed to play a major role in the transport of cholesterol from peripheral tissues to the liver for metabolism and excretion (this process is sometimes referred to as “reverse cholesterol transport”). ABCA1 is a transporter gene involved in HDL production and reverse cholesterol transport. Upregulation of ABCA1 can therefore result in increased reverse cholesterol transport as well as inhibition of cholesterol absorption in the gut. In addition, HDL is also believed to inhibit the oxidation of LDL cholesterol, reduce the inflammatory response of endothelial cells, inhibit the coagulation pathway, and promote the availability of nitric oxide.

Liver X receptors (LXRs), originally identified in the liver as orphan receptors, are members of the nuclear hormone receptor super family and are believed to be involved in the regulation of cholesterol and lipid metabolism. LXRs are ligand-activated transcription factors and bind to DNA as obligate heterodimers with retinoid X receptors. While LXRα is generally found in tissues such as liver, kidney, adipose tissue, intestine and macrophages, LXRβ displays a ubiquitous tissue distribution pattern. Activation of LXRs by oxysterols (endogenous ligands) in macrophages results in the expression of several genes involved in lipid metabolism and reverse cholesterol transport including the aforementioned ABCA1; ABCG1; and ApoE. See, e.g., Koldamova, et al., J. Biol. Chem. 2003, 278, 13244.

Studies have been conducted in LXRα knock-out (k/o), LXRβ k/o and double k/o mice to determine the physiological role of LXRs in lipid homeostasis and atherosclerosis. The data from these studies suggested that in double k/o mice on normal chow diet, increased cholesterol accumulation was observed in macrophages (foam cells) of the spleen, lung and arterial wall. The increased cholesterol accumulation was believed to be associated with the presence of reduced serum HDL cholesterol and increased LDL cholesterol, even though the total cholesterol levels in the mice were about normal. While LXRα k/o mice did not appear to show significant changes in hepatic gene expression, LXRβ k/o mice showed 58% decrease in hepatic ABCA1 expression and 208% increase in SREBP1c expression suggesting that LXRβ may be involved in the regulation of liver SREBP1c expression.

Data obtained from studies employing two different atherosclerotic mouse models (ApoE k/o and LDLR k/o) suggest that agonists of LXRα or β can be relatively effective in upregulating ABCA1 expression in macrophages. For example, inhibition of atherosclerotic lesions could be observed when ApoE k/o and LDLR k/o mice were treated with LXRα or β agonists for 12 weeks. The tested agonists were observed to have variable effects on serum cholesterol and lipoprotein levels and appeared to cause a relatively significant increase in serum HDL cholesterol and triglyceride levels. These in vivo data were found to be consistent with in vitro data obtained for the same agonists in macrophages.

In addition to the lipid and triglyceride effects described above, it is also believed that activation of LXRs results in the inhibition of inflammation and proinflammatory gene expression. This hypothesis is based on data obtained from studies employing three different models of inflammation (LPS-induced sepsis, acute contact dermatitis of the ear and chronic atherosclerotic inflammation of the artery wall). These data suggest that LXR modulators can mediate both the removal of cholesterol from the macrophages and the inhibition of vascular inflammation.

For a review of LXR biology and LXR modulators, see, e.g., Goodwin, et al., Current Topics in Medicinal Chemistry 2008, 8, 781; and Bennett, et al., Current Medicinal Chemistry 2008, 15, 195.

For studies related to atherosclerosis, see, e.g., Scott, J. N. Engl. J. Med. 2007, 357, 2195; Joseph, et al., PNAS 2002, 99, 7604; Tangirala, et. al., PNAS, 2002, 99, 11896; and Bradley, et al., Journal of Clinical Investigation 2007, 117, 2337-2346.

For studies related to inflammation, see, e.g., Fowler, et al., Journal of Investigative Dermatology 2003, 120, 246; and US 2004/0259948.

For studies related to Alzheimer's disease, see, e.g., Koldamova, et al., J. Biol. Chem. 2005, 280, 4079; Sun, et al., J. Biol. Chem. 2003, 278, 27688; and Riddell, et al., Mol. Cell. Neurosci. 2007, 34, 621.

For studies related to diabetes, see, e.g., Kase, et al., Diabetologia 2007, 50, 2171; and Liu, et al., Endocrinology 2006, 147, 5061.

For studies related to skin aging, see, e.g., WO 2004/076418; WO 2004/103320; and US 2008/0070883.

For studies related to arthritis, see, e.g., Chintalacharuvu, et. al., Arthritis a& Rheumatism 2007, 56, 1365; and WO 2008/036239.

### SUMMARY

This invention relates generally to quinoxaline-based modulators of Liver X receptors (LXRs) and related methods.

In one aspect, this invention features a compound having formula (I):

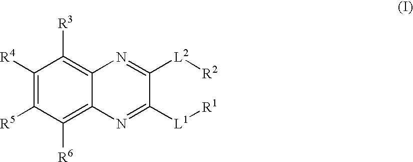

in which:

each of L¹ and L² is, independently, a bond, —O— or —NH—;

R¹ is:

(i) hydrogen; or

(ii) C₁-C₆ alkyl or C₁-C₆ haloalkyl, each of which is optionally substituted with from 1-3 R^{a}; or

(iii) C₂-C₆ alkenyl or C₂-C₆ alkynyl, each of which is optionally substituted with from 1-3 R^{b}; or

(iv) C₃-C₇ cycloalkyl optionally substituted with from 1-3 R^{c}; or

(v) NR⁷R⁸, wherein R⁷ and R⁸ at each occurrence is, independently, hydrogen, C₁-C₆ alkyl, or C₃-C₇ cycloalkyl;

R² is C₆-C₁₀ aryl or heteroaryl including 5-10 atoms, each of which is:

(i) substituted with 1 R⁹, and

(ii) optionally further substituted with from 1-4 R^{e}; wherein:

R⁹ is WA, wherein:

W at each occurrence is, independently, a bond; —O—; —NR¹⁰— wherein R¹⁰ is hydrogen, C₁-C₆ alkyl, or C₃-C₇ cycloalkyl; C₁₋₆ alkylene, C₂₋₆ alkenylene, or C₂₋₆ alkynylene; or —(C₁₋₆ alkylene)W¹—;

W¹ at each occurrence is, independently, —O—, —NH— or —N(C₁₋₆ alkyl)-;

A at each occurrence is, independently, C₆-C₁₀ aryl or heteroaryl including 5-10 atoms, each of which is:

(i) substituted with 1 R¹¹, and

(ii) optionally further substituted with from 1-4 R^{g};

R¹¹ at each occurrence is, independently:

(i) —W²—S(O)ₙR¹² or —W²—S(O)ₙNR¹³R¹⁴; or

(ii) —W²—C(O)OR¹⁵; or

(iii) —W²—C(O)NR¹³R¹⁴; or

(iv) —W²—CN; or

(v) C₁-C₈ alkyl or C₁-C₈ haloalkyl, each of which is:
(a) substituted with 1 R^{h}, and
(b) optionally further substituted with from 1-3 R^{a}; or

(vi) —NR¹⁶R¹⁷;

wherein:

W² at each occurrence is, independently, a bond; C₁₋₆ alkylene; C₂₋₆ alkenylene; C₂₋₆ alkynylene; C₃₋₆ cycloalkylene; —O(C₁₋₆ alkylene)-; —NH(C₁₋₆ alkylene)-; or —N(C₁-C₆ alkyl)(C₁₋₆ alkylene)-;

n at each occurrence is, independently, 1 or 2;

R¹² at each occurrence is, independently:

(i) C₁-C₆ alkyl or C₁-C₆ haloalkyl, each of which is optionally substituted with from 1-3 R^{a}; or

(ii) C₂-C₆ alkenyl or C₂-C₆ alkynyl, each of which is optionally substituted with from 1-3 R^{b}; or

(iii) C₃-C₈ cycloalkyl, C₃-C₈ cycloalkenyl, C₇-C₁₁ aralkyl, or heteroaralkyl including 6-11 atoms, each of which is optionally substituted with from 1-3 R^{c}; or

(iv) C₆-C₁₀ aryl or heteroaryl including 5-10 atoms, each of which is optionally substituted with from 1-3 R^{d};

R¹³ and R¹⁴ are each, independently, hydrogen; R¹² or heterocyclyl including 3-8 atoms or a heterocycloalkenyl including 3-10 atoms, each of which is optionally substituted with from 1-3 R^{c}; or

R¹³ and R¹⁴ together with the nitrogen atom to which they are attached form a heterocyclyl including 3-8 atoms or a heterocycloalkenyl including 3-8 atoms, each of which is optionally substituted with from 1-3 R^{c};

R¹⁵ at each occurrence is, independently, hydrogen or R¹²;

one of R¹⁶ and R¹⁷ is hydrogen or C₁-C₃ alkyl; and the other of R¹⁶ and R¹⁷ is:

(i) —S(O)ₙR¹²; or

(ii) —C(O)R¹²; or

(iii) —C(O)OR¹³; or

(iv) —C(O)NR¹³R¹⁴; or

(v) C₁-C₈ alkyl or C₁-C₈ haloalkyl, each of which is:
(a) substituted with 1 R^{h}, and
(b) optionally further substituted with from 1-3 R^{a};

each of R³ and R⁶ is, independently:

(i) hydrogen; or

(ii) halo; or

(iii) C₁-C₆ alkyl or C₁-C₆ haloalkyl, each of which is optionally substituted with from 1-3 R^{a}; or

(iv) nitro; hydroxy; C₁-C₆ alkoxy; C₁-C₆ haloalkoxy; C₁-C₆ thioalkoxy; C₁-C₆ thiohaloalkoxy; cyano; or S(O)_{z}(C₁-C₃ alkyl), wherein z is 1 or 2;

each of R⁴ and R⁵ is, independently:

(i) hydrogen; or

(ii) halo; or

(iii) C₁-C₆ alkyl or C₁-C₆ haloalkyl, each of which is optionally substituted with from 1-3 R^{a};

R^{a} at each occurrence is, independently:

(i) NR^{m}Rⁿ; hydroxy; C₁-C₆ alkoxy or C₁-C₆ haloalkoxy; or

(ii) C₃-C₇ cycloalkyl optionally substituted with from 1-3 substituents independently selected from NR^{m}Rⁿ; hydroxy; C₁-C₆ alkyl, C₁-C₆ alkoxy and C₁-C₆ haloalkoxy;

R^{b} at each occurrence is, independently:

(i) halo; NR^{m}Rⁿ; hydroxy; C₁-C₆ alkoxy or C₁-C₆ haloalkoxy; or

(ii) C₃-C₇ cycloalkyl optionally substituted with from 1-3 substituents independently selected from NR^{m}Rⁿ; hydroxy; C₁-C₆ alkyl, C₁-C₆ alkoxy and C₁-C₆ haloalkoxy;

R^{c} at each occurrence is, independently:

(i) halo; NR^{m}Rⁿ; hydroxy; C₁-C₆ alkoxy or C₁-C₆ haloalkoxy; or

(ii) C₁-C₆ alkyl or C₁-C₆ haloalkyl; or

(iii) C₂-C₆ alkenyl or C₂-C₆ alkynyl;

R^{d} at each occurrence is, independently:

(i) halo; NR^{m}Rⁿ; hydroxy; C₁-C₆ alkoxy or C₁-C₆ haloalkoxy; or cyano; or

(ii) C₁-C₆ alkyl or C₁-C₆ haloalkyl, each of which is optionally substituted with from 1-3 R^{a}; or

(iii) C₂-C₆ alkenyl or C₂-C₆ alkynyl, each of which is optionally substituted with from 1-3 R^{b};

R^{e} at each occurrence is, independently, C₁-C₆ alkyl; C₁-C₆ haloalkyl; halo; hydroxy; NR^{m}Rⁿ; C₁-C₆ alkoxy; C₁-C₆ haloalkoxy; cyano; or phenyl, which is optionally substituted with from 1-4 R^{d};

R^{g} at each occurrence is, independently, C₁-C₆ alkyl; C₁-C₆ haloalkyl; halo; hydroxy; NR^{m}Rⁿ; C₁-C₆ alkoxy; C₁-C₆ haloalkoxy; or cyano;

R^{h} at each occurrence is, independently, hydroxyl, C₁-C₆ alkoxy, or C₁-C₆ haloalkoxy; C₃-C₈ cycloalkoxy or C₃-C₈ cycloalkenyloxy, each of which is optionally substituted with from 1-3 R^{c}; or C₆-C₁₀ aryloxy or heteroaryloxy including 5-10 atoms, each of which is optionally substituted with from 1-3 R^{d};

each of R^{m} and Rⁿ at each occurrence is, independently, hydrogen, C₁-C₆ alkyl, or C₁-C₆ haloalkyl;

or an N-oxide and/or a pharmaceutically acceptable salt thereof.

In one aspect, this invention features a compound of formula (I-A):

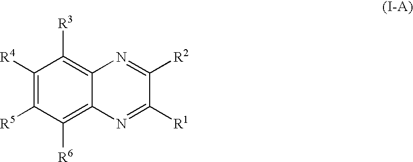

in which R¹, R², R³, R⁴, R⁵, and R⁶ can be as defined anywhere herein.

In one aspect, this invention relates to any subgenera of formula (I) or (I-A) described herein.

In one aspect, this invention relates to any of the specific quinoxaline compounds delineated herein. In some embodiments, the compound of formula (I) or (I-A) can be selected from the title compounds of Examples 1-42 and 44-83; or a pharmaceutically acceptable salt and/or N-oxide thereof.

In one aspect, this invention features a composition (e.g., a pharmaceutical composition), which includes a compound of formula (I) or (I-A) (including any subgenera or specific compounds thereof), or a salt (e.g., a pharmaceutically acceptable salt), or an N-oxide, or a prodrug thereof and a pharmaceutically acceptable carrier. In some embodiments, the composition can include an effective amount of the compound or the salt thereof. In some embodiments, the composition can further include an additional therapeutic agent.

In one aspect, this invention features a dosage form, which includes from about 0.05 milligrams to about 2,000 milligrams (e.g., from about 0.1 milligrams to about 1,000 milligrams, from about 0.1 milligrams to about 500 milligrams, from about 0.1 milligrams to about 250 milligrams, from about 0.1 milligrams to about 100 milligrams, from about 0.1 milligrams to about 50 milligrams, or from about 0.1 milligrams to about 25 milligrams) of formula (I) or (I-A) (including any subgenera or specific compounds thereof), or a salt (e.g., a pharmaceutically acceptable salt), or an N-oxide, or a prodrug thereof. The dosage form can further include a pharmaceutically acceptable carrier and/or an additional therapeutic agent.

The invention also relates generally to modulating (e.g., activating) LXRs with the quinoxaline compounds described herein. In some embodiments, the methods can include, e.g., contacting an LXR in a sample (e.g., a tissue, a cell free assay medium, a cell-based assay medium) with a compound of formula (I) or (I-A) (including any subgenera or specific compounds thereof) or a salt (e.g., a pharmaceutically acceptable salt), or an N-oxide, or a prodrug thereof. In other embodiments, the methods can include administering a compound of formula (I) (including any subgenera or specific compounds thereof) or a salt (e.g., a pharmaceutically acceptable salt), or an N-oxide, or a prodrug thereof to a subject (e.g., a mammal, e.g., a human, e.g., a human having or at risk of having one or more of the diseases or disorders described herein).

In one aspect, this invention also relates generally to methods of treating (e.g., controlling, relieving, ameliorating, alleviating, slowing the progression of, delaying the onset of, or reducing the risk of developing) or preventing one or more LXR-mediated diseases or disorders in a subject (e.g., a subject in need thereof). The methods include administering to the subject an effective amount of a compound of formula (I) or (I-A) (including any subgenera or specific compounds thereof) or a salt (e.g., a pharmaceutically acceptable salt), or an N-oxide, or a prodrug thereof. LXR-mediated diseases or disorders can include, e.g., cardiovascular diseases (e.g., acute coronary syndrome, restenosis, or coronary artery disease), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging or connective tissue diseases.

In another aspect, this invention relates to methods of modulating (e.g., increasing) serum HDL cholesterol levels in a subject (e.g., a subject in need thereof), which includes administering to the subject an effective amount of a compound of formula (I) or (I-A) (including any subgenera or specific compounds thereof) or a salt (e.g., a pharmaceutically acceptable salt), or an N-oxide, or a prodrug thereof.

In another aspect, this invention relates to methods of modulating (e.g., decreasing) serum LDL cholesterol levels in a subject (e.g., a subject in need thereof), which includes administering to the subject an effective amount of a compound of formula (I) or (I-A) (including any subgenera or specific compounds thereof) or a salt (e.g., a pharmaceutically acceptable salt), or an N-oxide, or a prodrug thereof.

In another aspect, this invention relates to methods of modulating (e.g., increasing) reverse cholesterol transport in a subject (e.g., a subject in need thereof), which includes administering to the subject an effective amount of a compound of formula (I) or (I-A) (including any subgenera or specific compounds thereof) or a salt (e.g., a pharmaceutically acceptable salt), or an N-oxide, or a prodrug thereof.

In another aspect, this invention relates to methods of modulating (e.g., decreasing or inhibiting) cholesterol absorption in a subject (e.g., a subject in need thereof), which includes administering to the subject an effective amount of a compound of formula (I) or (I-A) (including any subgenera or specific compounds thereof) or a salt (e.g., a pharmaceutically acceptable salt), or an N-oxide, or a prodrug thereof.

In a further aspect, this invention relates to methods of preventing or treating a cardiovascular disease (e.g., acute coronary syndrome, restenosis, or coronary artery disease), which includes administering to a subject in need thereof an effective amount of a compound of formula (I) or (I-A) (including any subgenera or specific compounds thereof) or a salt (e.g., a pharmaceutically acceptable salt), or an N-oxide, or a prodrug thereof.

In one aspect, this invention relates to methods of preventing or treating atherosclerosis and/or atherosclerotic lesions, which includes administering to a subject in need thereof an effective amount of a compound of formula (I) or (I-A) (including any subgenera or specific compounds thereof) or a salt (e.g., a pharmaceutically acceptable salt), or an N-oxide, or a prodrug thereof.

In another aspect, this invention relates to methods of preventing or treating diabetes (e.g., type I diabetes or type II diabetes), which includes administering to a subject in need thereof an effective amount of a compound of formula (I) or (I-A) (including any subgenera or specific compounds thereof) or a salt (e.g., a pharmaceutically acceptable salt), or an N-oxide, or a prodrug thereof.

In a further aspect, this invention relates to methods of preventing or treating Syndrome X, which includes administering to a subject in need thereof an effective amount of a compound of formula (I) or (I-A) (including any subgenera or specific compounds thereof) or a salt (e.g., a pharmaceutically acceptable salt), or an N-oxide, or a prodrug thereof.

In one aspect, this invention relates to methods of preventing or treating obesity, which includes administering to a subject in need thereof an effective amount of a compound of formula (I) or (I-A) (including any subgenera or specific compounds thereof) or a salt (e.g., a pharmaceutically acceptable salt), or an N-oxide, or a prodrug thereof.

In another aspect, this invention relates to methods of preventing or treating a lipid disorder (e.g., one or more of dyslipidemia, hyperlipidemia, hypertriglyceridemia, hypercholesterolemia, low HDL and/or high LDL), which includes administering to a subject in need thereof an effective amount of a compound of formula (I) or (I-A) (including any subgenera or specific compounds thereof) or a salt (e.g., a pharmaceutically acceptable salt), or an N-oxide, or a prodrug thereof.

In a further aspect, this invention relates to methods of preventing or treating a cognitive disorder (e.g., Alzheimer's disease or dementia), which includes administering to a subject in need thereof an effective amount of a compound of formula (I) or (I-A) (including any subgenera or specific compounds thereof) or a salt (e.g., a pharmaceutically acceptable salt), or an N-oxide, or a prodrug thereof.

In one aspect, this invention relates to methods of preventing or treating dementia, which includes administering to a subject in need thereof an effective amount of a compound of formula (I) or (I-A) (including any subgenera or specific compounds thereof) or a salt (e.g., a pharmaceutically acceptable salt), or an N-oxide, or a prodrug thereof.

In another aspect, this invention relates to methods of preventing or treating Alzheimer's disease, which includes administering to a subject in need thereof an effective amount of a compound of formula (I) or (I-A) (including any subgenera or specific compounds thereof) or a salt (e.g., a pharmaceutically acceptable salt), or an N-oxide, or a prodrug thereof.

In a further aspect, this invention relates to methods of preventing or treating an inflammatory disease (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), which includes administering to a subject in need thereof an effective amount of a compound of formula (I) or (I-A) (including any subgenera or specific compounds thereof) or a salt (e.g., a pharmaceutically acceptable salt), or an N-oxide, or a prodrug thereof.

In another aspect, this invention relates to methods of preventing or treating rheumatoid arthritis, which includes administering to a subject in need thereof an effective amount of a compound of formula (I) or (I-A) (including any subgenera or specific compounds thereof) or a salt (e.g., a pharmaceutically acceptable salt), or an N-oxide, or a prodrug thereof.

In a further aspect, this invention relates to methods of preventing or treating celiac, which includes administering to a subject in need thereof an effective amount of a compound of formula (I) or (I-A) (including any subgenera or specific compounds thereof) or a pharmaceutically acceptable salt or prodrug thereof.

In a further aspect, this invention relates to methods of preventing or treating thyroiditis, which includes administering to a subject in need thereof an effective amount of a compound of formula (I) or (I-A) (including any subgenera or specific compounds thereof) or a salt (e.g., a pharmaceutically acceptable salt), or an N-oxide, or a prodrug thereof.

In one aspect, this invention relates to methods of treating a connective tissue disease (e.g., osteoarthritis or tendonitis), which includes administering to a subject (e.g., a mammal, e.g., a human) in need thereof an effective amount of a compound of formula (I) or (I-A) (including any subgenera or specific compounds thereof) or a salt (e.g., a pharmaceutically acceptable salt), or an N-oxide, or a prodrug thereof. In embodiments, the compound of formula (I) or (I-A) inhibits (e.g., reduces or otherwise diminishes) cartilage degradation. In embodiments, the compound of formula (I) or (I-A) induces (e.g., increases or otherwise agments) cartilage regeneration. In embodiments, the compound of formula (I) or (I-A) inhibits (e.g., reduces or otherwise diminishes) cartilage degradation and induces (e.g., increases or otherwise agments) cartilage regeneration. In embodiments, the compound of formula (I) or (I-A) inhibits (e.g., reduces or otherwise diminishes) aggrecanase activity. In embodiments, the compound of formula (I) or (I-A) inhibits (e.g., reduces or otherwise diminishes) elaboration of pro-inflammatory cytokines in osteoarthritic lesions.

In another aspect, this invention relates to methods of treating or preventing skin aging, the method comprising administering (e.g., topically administering) to a subject (e.g., a mammal, e.g., a human) in need thereof an effective amount of a compound of formula (I) or (I-A) (including any subgenera or specific compounds thereof) or a salt (e.g., a pharmaceutically acceptable salt), or an N-oxide, or a prodrug thereof. In embodiments, the skin aging can be derived from chronological aging, photoaging, steroid-induced skin thinning, or a combination thereof.

The term “skin aging” includes conditions derived from intrinsic chronological aging (for example, deepened expression lines, reduction of skin thickness, inelasticity, and/or unblemished smooth surface), those derived from photoaging (for example, deep wrinkles, yellow and leathery surface, hardening of the skin, elastosis, roughness, dyspigmentations (age spots) and/or blotchy skin), and those derived from steroid-induced skin thinning. Accordingly, another aspect is a method of counteracting UV photodamage, which includes contacting a skin cell exposed to UV light with an effective amount of a compound of formula (I) or (I-A).

In some embodiments, the compound of formula (I) or (I-A) (including any subgenera or specific compounds thereof) or a salt (e.g., a pharmaceutically acceptable salt), or an N-oxide, or a prodrug thereof does not substantially increase serum and/or hepatic triglyceride levels of the subject.

In some embodiments, the administered compound of formula (I) or (I-A) (including any subgenera or specific compounds thereof) or a salt (e.g., a pharmaceutically acceptable salt), or an N-oxide, or a prodrug thereof can be an LXR agonist (e.g., an LXRα agonist or an LXRβ agonist).

In some embodiments, the subject can be a subject in need thereof (e.g., a subject identified as being in need of such treatment). Identifying a subject in need of such treatment can be in the judgment of a subject or a health care professional and can be subjective (e.g. opinion) or objective (e.g. measurable by a test or diagnostic method). In some embodiments, the subject can be a mammal. In certain embodiments, the subject is a human.

In a further aspect, this invention also relates to methods of making compounds described herein. Alternatively, the method includes taking any one of the intermediate compounds described herein and reacting it with one or more chemical reagents in one or more steps to produce a compound described herein.

In one aspect, this invention relates to a packaged product. The packaged product includes a container, one of the aforementioned compounds in the container, and a legend (e.g., a label or an insert) associated with the container and indicating administration of the compound for treatment and control of the diseases or disorders described herein.

In embodiments, any compound, composition, or method can also include any one or more of the features below (alone or in combination) and/or delineated in the detailed description and/or in the claims.

Each of L¹ and L² can be a bond. One of L¹ and L² (e.g., L¹) can be a bond, and the other of L¹ and L² (e.g., L²) is —O—.

R¹ can be C₁-C₆ alkyl or C₁-C₆ haloalkyl. In certain embodiments, R¹ can be C₁-C₃ alkyl (e.g., CH₃).

In some embodiments, when L¹ is —NH— or —O—, then R¹ can be other than hydrogen and/or other than NR⁷R⁸.

R² can be C₆-C₁₀ aryl, which is (a) substituted with 1 R⁹; and (b) optionally further substituted with from 1-4 R^{e}.

In certain embodiments, R² can be phenyl, which is (a) substituted with 1 R⁹; and (b) optionally further substituted with from 1-4 R^{e}. For example, R² can have formula (A-2):

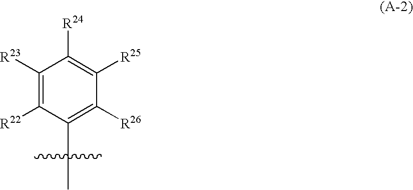

in which:

one of R²³ and R²⁴ is R⁹, and the other of R²³ and R²⁴ is hydrogen, and each of R²², R²⁵, and R²⁶ is, independently, hydrogen or R^{e}.

R²³ can be R⁹, and R²⁴ can be hydrogen. R²⁴ can be R⁹, and R²³ can be hydrogen. Each of R²², R²⁵, and R²⁶ can be hydrogen. One of R²², R²⁵, and R²⁶ (e.g., R²⁶) can be R^{e} (e.g., halo, e.g., chloro) and the other two are hydrogen.

W can be —O—.

W can be a bond.

A can be C₆-C₁₀ aryl, which is (a) substituted with 1 R¹¹; and (b) optionally further substituted with from 1-4 R^{g}.

In certain embodiments, A can be phenyl, which is (a) substituted with 1 R¹¹; and (b) optionally further substituted with from 1-4 R^{g}. For example, A can have formula (B-1):

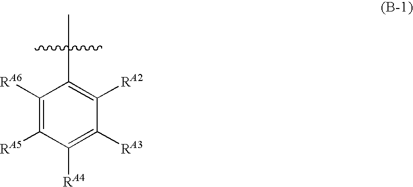

in which:

one of R^{A3} and R^{A4} is R¹¹, the other of R^{A3} and R^{A4} is hydrogen; and each of R^{A2}, R^{A5}, and R^{A6} is, independently, hydrogen or R^{g}.

R¹¹ can be —W²—S(O)ₙR¹². In embodiments, W² can be a bond, and n is 2. R¹² can be C₁-C₆ alkyl, optionally substituted with from 1-2 R^{a}. For example, R¹² can be CH₃.

R² can have formula (A-2) as described herein, in which:

one of R²³ and R²⁴ can have formula (C-1):

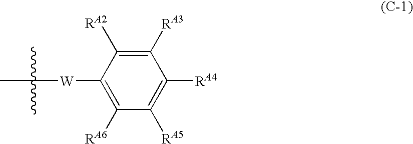

in which one of R^{A2}, R^{A3}, R^{A4}, R^{A5}, and R^{A6} is R¹¹, and the others are each, independently, hydrogen or R^{g}; and the other of R²³ and R²⁴ can be hydrogen; and each of R²², R²⁵, and R²⁶ is, independently, hydrogen or R^{e}.

Embodiments can include, for example, one or more of the following features.

R²³ can have formula (C-1), and R²⁴ can be hydrogen. Each of R²², R²⁵, and R²⁶ can be hydrogen. One of R²², R²⁵, and R²⁶ (e.g., R²⁶) can be R^{e} (e.g., halo, e.g., chloro) and the other two are hydrogen.

W can be —O—. W can be a bond.

One of R^{A3} and R^{A4} can be R¹¹, and the other of R^{A3} and R^{A4} is hydrogen; and each of R^{A2}, R^{A5}, and R^{A6} is, independently, hydrogen or R^{g}.

R¹¹ can be —W²—S(O)ₙR¹².

R^{A3} can be R¹¹, and R^{A4} is hydrogen. R¹¹ can be —W²—S(O)ₙR¹². W² can be a bond, and n can be 2. R¹² can be C₁-C₆ alkyl, optionally substituted with from 1-2 R^{a}. For example, R¹² can be CH₃. R¹² can be C₁-C₆ alkyl substituted with 1 R^{a}. R^{a} can be, e.g., hydroxyl or NR^{m}Rⁿ.

Each of R^{A2}, R^{A5}, and R^{A6} can be hydrogen. R^{A5} can be R^{g} (e.g., halo), and each of R^{A2} and R^{A6} is hydrogen.

R¹¹ can be —W²—S(O)ₙNR¹³R¹⁴. W² can be a bond, and one of R¹³ and R¹⁴ can be C₁-C₃ alkyl, and the other of R¹³ and R¹⁴ can be hydrogen.

One of R³ and R⁶ can be:

(i) halo; or (ii) C₁-C₆ alkyl or C₁-C₆ haloalkyl, each of which is optionally substituted with from 1-3 R^{a}; or (iii) nitro; hydroxy; C₁-C₆ alkoxy; C₁-C₆ haloalkoxy; C₁-C₆ thioalkoxy; C₁-C₆ thiohaloalkoxy; cyano; or S(O)_{z}(C₁-C₃ alkyl), wherein z is 1 or 2;

and the other of R³ and R⁶ can be:

(i) hydrogen; or (ii) halo; or (iii) C₁-C₆ alkyl or C₁-C₆ haloalkyl, each of which is optionally substituted with from 1-3 R^{a}; or (iv) nitro; hydroxy; C₁-C₆ alkoxy; C₁-C₆ haloalkoxy; C₁-C₆ thioalkoxy; C₁-C₆ thiohaloalkoxy; cyano; or S(O)_{z}(C₁-C₃ alkyl), wherein z is 1 or 2.

One of R³ and R⁶ can be:

(i) halo; or (ii) C₁-C₆ alkyl or C₁-C₆ haloalkyl, each of which is optionally substituted with from 1-3 R^{a}; or (iii) nitro; hydroxy; C₁-C₆ alkoxy; C₁-C₆ haloalkoxy; C₁-C₆ thioalkoxy; C₁-C₆ thiohaloalkoxy; cyano; or S(O)_{z}(C₁-C₃ alkyl), wherein z is 1 or 2;

and the other of R³ and R⁶ can be hydrogen.

One of R³ and R⁶ can be:

(i) halo; or (ii) C₁-C₆ haloalkyl; or (iii) C₁-C₆ alkoxy; C₁-C₆ haloalkoxy; or cyano;

and the other of R³ and R⁶ can be hydrogen.

One of R³ and R⁶ can be halo (e.g., chloro), the other of R³ and R⁶ is hydrogen.

One of R³ and R⁶ can be C₁-C₄ haloalkyl (e.g., perfluoroalkyl, e.g., CF₃), the other of R³ and R⁶ can be hydrogen.

Each of R⁴ and R⁵ can be hydrogen.

In some embodiment, the compound can have formula (VI):

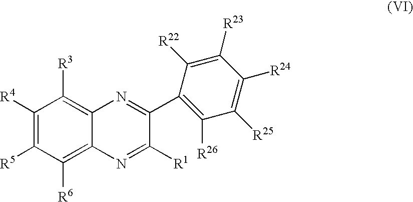

in which:

R¹ is:

(i) hydrogen; or

(ii) C₁-C₃ alkyl or C₁-C₃ haloalkyl; or

(iii) NR⁷R⁸;

one of R³ and R⁶ is:

(i) halo; or

(ii) C₁-C₄ haloalkyl; or

(iii) C₁-C₆ alkoxy; C₁-C₆ haloalkoxy; or cyano;

and the other of R³ and R⁶ is hydrogen;

each of R⁴ and R⁵ is hydrogen;

one of R²³ and R²⁴ can have formula (C-1) as described herein, in which one of R^{A2}, R^{A3}, R^{A4}, R^{A5}, and R^{A6} can be R¹¹, and the others can each be, independently, hydrogen or R^{g}; and W is a bond or —O—;

and the other of R²³ and R²⁴ is hydrogen, and

each of R²², R²⁵, and R²⁶ is, independently, hydrogen or R^{e}.

Embodiments can include, for example, one or more of the following features.

R¹ can be CH₃.

R²³ can have formula (C-1), and R²⁴ can be hydrogen. Each of R²², R²⁵, and R²⁶ can be hydrogen. One of R²², R²⁵, and R²⁶ (e.g., R²⁶) can be R^{e} (e.g., halo, e.g., chloro) and the other two are hydrogen.

One of R^{A3} and R^{A4} can be R¹¹, and the other of R^{A3} and R^{A4} can be hydrogen; and each of R^{A2}, R^{A5}, and R^{A6} can be, independently, hydrogen or R^{g}. R^{A3} can be R¹¹, and R^{A4} can be hydrogen. R¹¹ can be —W²—S(O)ₙR¹². W² can be a bond, and n can be 2. R¹² can be C₁-C₆ alkyl, optionally substituted with from 1-2 R^{a} (e.g., CH₃). Each of R^{A2}, R^{A5}, and R^{A6} can be hydrogen; or R^{A5} can be R^{g}, and each of R^{A2} and R^{A6} can be hydrogen. R¹¹ can be —W²—S(O)ₙNR¹³R¹⁴, in which W² can be a bond, and one of R¹³ and R¹⁴ is C₁-C₃ alkyl, and the other of R¹³ and R¹⁴ is hydrogen.

R³ can be hydrogen, and R⁶ can be CF₃.

R³ can be CF₃, and R⁶ can be hydrogen.

The term “mammal” includes organisms, which include mice, rats, cows, sheep, pigs, rabbits, goats, horses, monkeys, dogs, cats, and humans.

“An effective amount” refers to an amount of a compound that confers a therapeutic effect (e.g., treats, inhibits, controls, relieves, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect). An effective amount of the compound described above may range from about 0.01 mg/kg to about 1000 mg/kg, (e.g., from about 0.1 mg/kg to about 100 mg/kg, from about 1 mg/kg to about 100 mg/kg). Effective doses will also vary depending on route of administration, as well as the possibility of co-usage with other agents.

The term “halo” or “halogen” refers to any radical of fluorine, chlorine, bromine or iodine.

In general, and unless otherwise indicated, substituent (radical) prefix names are derived from the parent hydride by either (i) replacing the “ane” in the parent hydride with the suffixes “yl,” “diyl,” “triyl,” “tetrayl,” etc.; or (ii) replacing the “e” in the parent hydride with the suffixes “yl,” “diyl,” “triyl,” “tetrayl,” etc. (here the atom(s) with the free valence, when specified, is (are) given numbers as low as is consistent with any established numbering of the parent hydride). Accepted contracted names, e.g., adamantyl, naphthyl, anthryl, phenanthryl, furyl, pyridyl, isoquinolyl, quinolyl, and piperidyl, and trivial names, e.g., vinyl, allyl, phenyl, and thienyl are also used herein throughout. Conventional numbering/lettering systems are also adhered to for substituent numbering and the nomenclature of fused, bicyclic, tricyclic, polycyclic rings.

The term “alkyl” refers to a saturated hydrocarbon chain that may be a straight chain or branched chain, containing the indicated number of carbon atoms. For example, C₁-C₆ alkyl indicates that the group may have from 1 to 6 (inclusive) carbon atoms in it. Any atom can be optionally substituted, e.g., by one or more substitutents (e.g., such as those delineated in any definition of R^{a} described herein). Examples of alkyl groups include without limitation methyl, ethyl, n-propyl, isopropyl, and tert-butyl.

The terms “alkylene,” “alkenylene,” “alkynylene,” and “cycloalkylene” refer to divalent straight chain or branched chain alkyl (e.g., —CH₂—), alkenyl (e.g., —CH═CH—), alkynyl (e.g., —C≡C—); or cycloalkyl moieties, respectively.

The term “haloalkyl” refers to an alkyl group, in which at least one hydrogen atom is replaced by halo. In some embodiments, more than one hydrogen atom (e.g., 2, 3, 4, 5, 6, 7, 8, 9, 10, 11, 12, 13, or 14) are replaced by halo. In these embodiments, the hydrogen atoms can each be replaced by the same halogen (e.g., fluoro) or the hydrogen atoms can be replaced by a combination of different halogens (e.g., fluoro and chloro). “Haloalkyl” also includes alkyl moieties in which all hydrogens have been replaced by halo (sometimes referred to herein as perhaloalkyl, e.g., perfluoroalkyl, such as trifluoromethyl). Any atom can be optionally substituted, e.g., by one or more substituents (e.g., such as those delineated in any definition of R^{a} described herein).

The term “aralkyl” refers to an alkyl moiety in which an alkyl hydrogen atom is replaced by an aryl group. One of the carbons of the alkyl moiety serves as the point of attachment of the aralkyl group to another moiety. Any ring or chain atom can be optionally substituted e.g., by one or more substituents (e.g., such as those delineated in any definition of R^{c} described herein). Non-limiting examples of “aralkyl” include benzyl, 2-phenylethyl, and 3-phenylpropyl groups.

The term “heteroaralkyl” refers to an alkyl moiety in which an alkyl hydrogen atom is replaced by a heteroaryl group. One of the carbons of the alkyl moiety serves as the point of attachment of the aralkyl group to another moiety. Heteroaralkyl includes groups in which more than one hydrogen atom on an alkyl moiety has been replaced by a heteroaryl group. Any ring or chain atom can be optionally substituted e.g., by one or more substituents (e.g., such as those delineated in any definition of R^{c} described herein). Heteroaralkyl can include, for example, 2-pyridylethyl.

The term “alkenyl” refers to a straight or branched hydrocarbon chain containing the indicated number of carbon atoms and having one or more carbon-carbon double bonds. Any atom can be optionally substituted, e.g., by one or more substituents (e.g., such as those delineated in any definition of R^{b} described herein). Alkenyl groups can include, e.g., allyl, 1-butenyl, and 2-hexenyl. One of the double bond carbons can optionally be the point of attachment of the alkenyl substituent. The term “alkynyl” refers to a straight or branched hydrocarbon chain containing the indicated number of carbon atoms and having one or more carbon-carbon triple bonds. Any atom can be optionally substituted, e.g., by one or more substituents (e.g., such as those delineated in any definition of R^{b} described herein). Alkynyl groups can include, e.g., ethynyl, propargyl, and 3-hexynyl. One of the triple bond carbons can optionally be the point of attachment of the alkynyl substituent.

The term “alkoxy” refers to an —O-alkyl radical. The term “mercapto” refers to an SH radical. The term “thioalkoxy” refers to an —S-alkyl radical. The terms “aryloxy” and “heteroaryloxy” refer to an —O-aryl radical and —O-heteroaryl radical, respectively. The terms “thioaryloxy” and “thioheteroaryloxy” refer to an —S-aryl radical and —S-heteroaryl radical, respectively.

The terms “aralkoxy” and “heteroaralkoxy” refer to an —O-aralkyl radical and —O-heteroaralkyl radical, respectively. The terms “thioaralkoxy” and “thioheteroaralkoxy” refer to an —S-aralkyl radical and —S-heteroaralkyl radical, respectively. The term “cycloalkoxy” refers to an —O-cycloalkyl radical. The terms “cycloalkenyloxy” and “heterocycloalkenyloxy” refer to an —O-cycloalkenyl radical and —O-heterocycloalkenyl radical, respectively. The term “heterocyclyloxy” refers to an —O-heterocyclyl radical. The term “thiocycloalkoxy” refers to an —S-cycloalkyl radical. The terms “thiocycloalkenyloxy” and “thioheterocycloalkenyloxy” refer to an —S-cycloalkenyl radical and —S-heterocycloalkenyl radical, respectively. The term “thioheterocyclyloxy” refers to an —S-heterocyclyl radical.

The term “heterocyclyl” refers to a fully saturated monocyclic, bicyclic, tricyclic or other polycyclic ring system having one or more (e.g., 1-4) heteroatom ring atoms independently selected from O, N, or S. The heteroatom or ring carbon is the point of attachment of the heterocyclyl substituent to another moiety. Any atom can be optionally substituted, e.g., by one or more substituents (e.g., such as those delineated in any definition of R^{c} described herein). Heterocyclyl groups can include, e.g., tetrahydrofuryl, tetrahydropyranyl, piperidyl (piperidino), piperazinyl, morpholinyl (morpholino), pyrrolinyl, and pyrrolidinyl.

The term “heterocycloalkenyl” refers to partially unsaturated monocyclic, bicyclic, tricyclic, or other polycyclic hydrocarbon groups having one or more (e.g., 1-4) heteroatom ring atoms independently selected from O, N, or S. A ring carbon (e.g., saturated or unsaturated) or heteroatom is the point of attachment of the heterocycloalkenyl substituent. Any atom can be optionally substituted, e.g., by one or more substituents (e.g., such as those delineated in any definition of R^{c} described herein). Heterocycloalkenyl groups can include, e.g., tetrahydropyridyl, dihydropyranyl, 4,5-dihydrooxazolyl, 4,5-dihydro-1H-imidazolyl, 1,2,5,6-tetrahydro-pyrimidinyl, and 5,6-dihydro-2H-[1,3]oxazinyl.

The term “cycloalkyl” refers to a fully saturated monocyclic, bicyclic, tricyclic, or other polycyclic hydrocarbon groups. Any atom can be optionally substituted, e.g., by one or more substituents (e.g., such as those delineated in any definition of R^{c} described herein). A ring carbon serves as the point of attachment of a cycloalkyl group to another moiety. Cycloalkyl moieties can include, e.g., cyclopropyl, cyclobutyl, cyclopentyl, cyclohexyl, cycloheptyl, adamantyl, and norbornyl (bicycle[2.2.1]heptyl).

The term “cycloalkenyl” refers to partially unsaturated monocyclic, bicyclic, tricyclic, or other polycyclic hydrocarbon groups. A ring carbon (e.g., saturated or unsaturated) is the point of attachment of the cycloalkenyl substituent. Any atom can be optionally substituted e.g., by one or more substituents (e.g., such as those delineated in any definition of R^{c} described herein). Cycloalkenyl moieties can include, e.g., cyclohexenyl, cyclohexadienyl, or norbornenyl.

The term “aryl” refers to an aromatic monocyclic or bicyclic hydrocarbon ring system, wherein any ring atom can be optionally substituted, e.g., by one or more substituents (e.g., such as those delineated in any definition of R^{d} described herein). Aryl moieties include phenyl and naphthyl.

The term “heteroaryl” refers to an aromatic monocyclic or bicyclic hydrocarbon groups having one or more (e.g., 1-6) heteroatom ring atoms independently selected from O, N, or S (and mono and dioxides thereof, e.g., N→O⁻, S(O), SO₂). Any atom can be optionally substituted, e.g., by one or more substituents (e.g., such as those delineated in any definition of R^{d} described herein). Heteroaryl groups include pyridyl, thienyl, furyl (furanyl), imidazolyl, isoquinolyl, quinolyl and pyrrolyl.

The descriptor C(O) refers to a carbon atom that is doubly bonded to an oxygen atom.

The term “substituent” refers to a group “substituted” on, e.g., an alkyl, haloalkyl, cycloalkyl, alkenyl, alkynyl, aralkyl, heteroaralkyl, heterocyclyl, heterocycloalkenyl, cycloalkenyl, aryl, or heteroaryl group at any atom of that group. In one aspect, the substituent(s) (e.g., R^{a}) on a group are independently any one single, or any combination of two or more of the permissible atoms or groups of atoms delineated for that substituent. In another aspect, a substituent may itself be substituted with any one of the above substituents.

In general, when a definition for a particular variable includes both hydrogen and non-hydrogen (halo, alkyl, aryl, etc.) possibilities, the term “substituent(s) other than hydrogen” refers collectively to the non-hydrogen possibilities for that particular variable.

Descriptors such as “C₁-C₆ which is optionally substituted with from 1-5 R^{a}” (and the like) is intended to include both an unsubstituted C₁-C₆ alkyl group and a C₁-C₆ alkyl group that is substituted with from 1-5 R^{a}. The use of a substituent (radical) prefix names such as alkyl without the modifier “optionally substituted” or “substituted” is understood to mean that the particular substituent is unsubstituted. However, the use of “haloalkyl” without the modifier “optionally substituted” or “substituted” is still understood to mean an alkyl group, in which at least one hydrogen atom is replaced by halo.

In some embodiments, the compounds have agonist activity for genes involved with HDL production and cholesterol efflux (e.g., ABCA1) and antagonist activity for genes involved with triglyceride synthesis (e.g., SREBP-1c).

The details of one or more embodiments of the invention are set forth in the description below. Other features and advantages of the invention will be apparent from the description and from the claims.

### DETAILED DESCRIPTION

This invention relates generally to quinoxaline-based modulators of Liver X receptors (LXRs) and related methods.

The quinoxaline-based LXR modulators have the general formula (I):

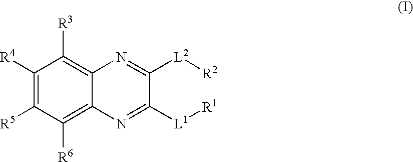

Here and throughout this specification, L¹, L², R¹, R², R³, R⁴, R⁵, R⁶, R⁷, R⁸, R⁹, R¹⁰, R¹¹, R¹², R¹³, R¹⁴, R¹⁵, R¹⁶, R¹⁷, W, W¹, W², A, R^{a}, R^{b}, R^{c}, R^{d}, R^{e}, R^{g}, R^{h}, R^{m}, Rⁿ, z, and n can be as defined anywhere herein.

In some embodiments, the quinoxaline-based LXR modulators have formula (I-A) as described herein. Here and throughout this specification, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, W, W¹, W², A, R^{a}, R^{b}, R^{c}, R^{d}, R^{e}, R^{g}, R^{h}, R^{m}, Rⁿ, z, and n can be as defined anywhere herein.

For ease of exposition, it is also understood that where in this specification (including the claims), a group is defined by “as defined anywhere herein” (or the like), the definitions for that particular group include the first occurring and broadest generic definition as well as any sub-generic and specific definitions delineated anywhere in this specification.

Variables L¹ and L²

In some embodiments, each of L¹ and L² can be a bond.

In some embodiments, one of L¹ and L² can be a bond. In certain embodiments, L¹ can be a bond. In certain embodiments, one of L¹ and L² (e.g., L¹) can be a bond, and the other of L¹ and L² (e.g., L¹) can be —O— or NH (e.g., —O—).

In some embodiments, when L¹ is —NH— or —O—, then R¹ can be other than hydrogen and/or other than NR⁷R⁸.

Variable R¹

In some embodiments, R¹ can be:

(1-i) hydrogen; or

(1-ii) C₁-C₆ (e.g., C₁-C₃) alkyl or C₁-C₆ (e.g., C₁-C₄ or C₁-C₃) haloalkyl, each of which is optionally substituted with from 1-3 (e.g., 1-2, 1) R^{a}; or

(1-iii) C₂-C₆ alkenyl or C₂-C₆ alkynyl, each of which is optionally substituted with from 1-3 (e.g., 1-2, 1) R^{b}; or

(1-iv) C₃-C₇ (e.g., C₃-C₆) cycloalkyl, which is optionally substituted with from 1-3 (e.g., 1-2, 1) R^{c}; or

(1-v) NR⁷R⁸, wherein R⁷ and R⁸ at each occurrence is, independently, hydrogen, C₁-C₆ (e.g., C₁-C₃) alkyl, or C₃-C₇ (e.g., C₃-C₆) cycloalkyl;

In some embodiments, R¹ can be:

(1-ii) C₁-C₆ (e.g., C₁-C₃) alkyl or C₁-C₆ (e.g., C₁-C₄ or C₁-C₃) haloalkyl, each of which is optionally substituted with from 1-3 (e.g., 1-2, 1) R^{a}; or

(1-iii) C₂-C₆ alkenyl or C₂-C₆ alkynyl, each of which is optionally substituted with from 1-3 (e.g., 1-2, 1) R^{b}; or

(1-iv) C₃-C₇ (e.g., C₃-C₆) cycloalkyl, which is optionally substituted with from 1-3 (e.g., 1-2, 1) R^{c}; or

(1-v) NR⁷R⁸, wherein R⁷ and R⁸ at each occurrence is, independently, hydrogen, C₁-C₆ (e.g., C₁-C₃) alkyl, or C₃-C₇ (e.g., C₃-C₆) cycloalkyl;

In some embodiments, R¹ can be hydrogen.

In certain embodiments, R¹ can be (1-i), (1-iia) C₁-C₆ (e.g., C₁-C₃) alkyl, which is optionally substituted with from 1-3 (e.g., 1-2, 1) R^{a}; (1-iii); (1-iv); or (1-v).

In certain embodiments, R¹ can be (1-i), (1-iib) C₁-C₆ (e.g., C₁-C₃) haloalkyl, which is optionally substituted with from 1-3 (e.g., 1-2, 1) R^{a}; (1-iii); (1-iv); or (1-v).

In embodiments, R¹ can be other than (1-v).

In some embodiments, when L¹ is —NH— or —O—, then R¹ can be other than hydrogen and/or other than NR⁷R⁸.

In some embodiments, R¹ can be any one of: (1-i), (1-ii), (1-iia), (1-iib), (1-iii), (1-iv), and (1-v). In certain embodiments, R¹ can be hydrogen. In other embodiments, R¹ can be a substituent other than hydrogen. For example, R¹ can be C₁-C₆ (e.g., C₁-C₃) alkyl or C₁-C₆ (e.g., C₁-C₄) haloalkyl, each of which is optionally substituted with from 1-3 (e.g., 1-2, 1) R^{a}; e.g., C₁-C₆ (e.g., C₁-C₃) alkyl, which is optionally substituted with from 1-3 (e.g., 1-2, 1) R^{a}.

In some embodiments, R¹ can be any two of: (1-i), (1-ii), (1-iia), (1-iib), (1-iii), (1-iv), and (1-v). In certain embodiments, R¹ can be hydrogen and any one of (1-i), (1-ii), (1-iia), (1-iib), (1-iii), (1-iv), and (1-v). In other embodiments, R¹ can be any two of (1-ii), (1-iia), (1-iib), (1-iii), (1-iv), and (1-v), e.g., any two of (1-ii), (1-iia), (1-iib), (1-iii), and (1-iv).

In some embodiments, R¹ can be any three of: (1-i), (1-ii), (1-iia), (1-iib), (1-iii), (1-iv), and (1-v). In certain embodiments, R¹ can be hydrogen and any two of (1-ii), (1-iia), (1-iib), (1-iii), (1-iv), and (1-v); e.g., any two of (1-ii), (1-iia), (1-iib), (1-iii), and (1-iv). In other embodiments, R¹ can be any three of (1-ii), (1-iia), (1-iib), (1-iii), (1-iv), and (1-v); any three of (1-ii), (1-iia), (1-iib), (1-iii), and (1-iv).

In some embodiments, R¹ can be C₁-C₆ (e.g., C₁-C₃) alkyl, which is optionally substituted with from 1-3 (e.g., 1-2, 1) R^{a}. In certain embodiments, R¹ can be C₁-C₆ (e.g., C₁-C₃) alkyl. For example, R¹ can be methyl (CH₃), ethyl (CH₂CH₃), or isopropyl (CH(CH₃)₂). In certain embodiments, R¹ can be methyl (CH₃).

In some embodiments, R¹ can be C₁-C₆ (e.g., C₁-C₄ or C₁-C₃) haloalkyl (e.g., perhaloalkyl). For example, R¹ can be CF₃.

In some embodiments, R¹ can be NR⁷R⁸, in which R⁷ and R⁸ at each occurrence can be, independently, hydrogen or (e.g., C₁-C₃) C₁-C₆ alkyl. For example, each of R⁷ and R⁸ can be hydrogen.

Variable R²

In some embodiments, R² can be C₆-C₁₀ (e.g., phenyl) aryl, which is (i) substituted with 1 R⁹ and (ii) optionally substituted with from 1-4 (e.g., 1-3, 1-2, 1) R^{e}. In these and in the following embodiments related to variables R², R⁹ and R^{e} can be as defined anywhere herein.

In some embodiments, R² can be C₆-C₁₀ aryl, which is (i) substituted with 1 R⁹ and (ii) optionally substituted with 1 or 2 R^{e}.

In embodiments, when R² is aryl and substituted with one (or more) R^{e}, each R^{e} can be independently of one another: halo (e.g., chloro); C₁-C₃ alkyl; C₁-C₃ haloalkyl (e.g., C₁-C₃ fluoroalkyl, e.g., 1-5 fluorines can be present; or C₁-C₃ perfluoroalkyl); CN; hydroxyl; NR^{m}Rⁿ (e.g., NH₂, monoalkylamino, or dialkylamino, in which each alkyl portion can independently include, e.g., from 1-3 carbon atoms); C₁-C₃ alkoxy; C₁-C₃ haloalkoxy, or C₆-C₁₀ aryl (e.g., phenyl) which is optionally substituted with from 1-4 (e.g., 1-3, 1-2, or 1) R^{d}.

In certain embodiments, when R² is aryl and substituted with R^{e}, each R^{e} can be independently of one another: C₁-C₃ alkyl; C₁-C₃ haloalkyl, e.g., C₁-C₃ perfluoroalkyl; halo (e.g., fluoro or chloro); CN, or phenyl which is optionally substituted with from 1-5 (e.g., 1-3, 1-2, or 1) R^{d}.

In certain embodiments, when R² is aryl and substituted with R^{e}, each R^{e} can be independently of one another: C₁-C₃ alkyl; C₁-C₃ haloalkyl, e.g., C₁-C₃ perfluoroalkyl; halo (e.g., fluoro or chloro), or phenyl which is optionally substituted with from 1-5 (e.g., 1-3, 1-2, or 1) R^{d}.

In certain embodiments, when R² is aryl and substituted with R^{e}, each R^{e} can be independently of one another halo (e.g., fluoro or chloro).

In certain embodiments, R² can be phenyl, which is (i) substituted with 1 R⁹ and (ii) optionally substituted with from 1-4 (e.g., 1-3, 1-2, or 1) R^{e} (e.g., halo, e.g., fluoro or chloro). In other embodiments, R² can be phenyl, which is substituted only with 1 R⁹.

In certain embodiments, R² can have formula (A), in which R⁹ (i.e., the moiety —WA) can be attached to a ring carbon that is ortho, meta, or para (e.g., meta or para, e.g., meta) with respect to the ring carbon that connects the phenyl ring to the 2- or 3-position of the quinoxalinering, and R^{e}, when present can be connected to ring carbons that are not occupied by WA. For example, R² can have formula (A-1), in which R⁹ (WA) is attached to the ring carbon that is meta with respect to the ring carbon that connects the phenyl ring to the 2- or 3-position of the quinoxalinering in formula (I).

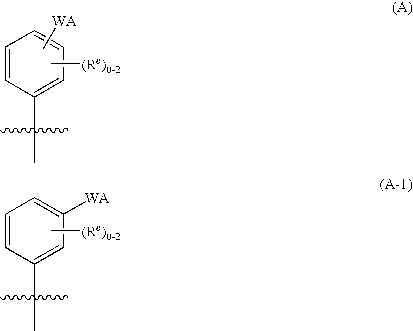

In certain embodiments, R² can have formula (A-2):

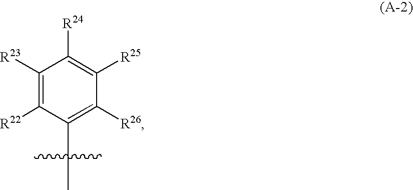

in which one of R²³ and R²⁴ (e.g., R²³) is R⁹, and the other of R²³ and R²⁴ (e.g., R²⁴) is hydrogen, and each of R²², R²⁵, and R²⁶ is, independently, hydrogen or R^{e}.

In embodiments, each of R²², R²⁵, and R²⁶ can be hydrogen. In other embodiments, each of R²², R²⁵, and R²⁶ can be a substituent other than hydrogen. In still other embodiments, one or two of R²², R²⁵, and R²⁶ can be R^{e}, and the other(s) are hydrogen.

In certain embodiments, one of R²², R²⁵, and R²⁶ can be R^{e}, and the other two are hydrogen. In embodiments, R²⁶ can be R^{e}, and each of R²² and R²⁵ can be hydrogen. In these embodiments, R^{e} can be: halo (e.g., chloro or fluoro, e.g., chloro); C₁-C₃ alkyl; C₁-C₃ haloalkyl (e.g., C₁-C₃ fluoroalkyl, e.g., 1-5 fluorines can be present; or C₁-C₃ perfluoroalkyl); or C₆-C₁₀ aryl (e.g., phenyl) which is optionally substituted with from 1-5 R^{d}. For example, R^{e} can be halo (e.g., fluoro or chloro). In other embodiments, R^{e} can be phenyl, which is optionally substituted with from 1-4 R^{d}.

In some embodiments, R² can be heteroaryl including 5-10 (e.g., 5-6) atoms, which is (i) substituted with 1 R⁹ and (ii) optionally substituted with from 1-4 (e.g., 1-3, 1-2, 1) R^{e}. In these and in the following embodiments related to variable R², R⁹ and R^{e} can be as defined anywhere herein.

In some embodiments, R² can be heteroaryl including 5-10 atoms, which is (i) substituted with 1 R⁹ and (ii) optionally substituted with 1 or 2 R^{e}.

In embodiments, when R² is heteroaryl and substituted with R^{e}, each R^{e} can be independently as defined anywhere herein. For example, each R^{e} can be independently of one another: C₁-C₃ alkyl; C₁-C₃ haloalkyl, e.g., C₁-C₃ perfluoroalkyl; halo (e.g., chloro); e.g., each R^{e} can be halo (e.g., chloro).

In some embodiments, R² can be heteroaryl including 5-6 atoms, which is (i) substituted with 1 R⁹ and (ii) optionally substituted with 1 or 2 R^{e}.

In some embodiments, R² can be heteroaryl including 8-10 atoms, which is (i) substituted with 1 R⁹ and (ii) optionally substituted with 1 or 2 R^{e}.

In certain embodiments, R² can be pyridyl, pyrimidinyl, thienyl, furyl, quinolinyl, oxazolyl, thiazolyl, imidazolyl, isoxazolyl, indolyl, benzo[1,3]-dioxolyl, benzo[1,2,5]-oxadiazolyl, isochromenyl-1-one, 3-H-isobenzofuranyl-1-one (e.g., pyridyl, thienyl, or indolyl, e.g., pyridyl), each of which is (i) substituted with 1 R⁹ and (ii) optionally substituted with 1 or 2 R^{e}. For example, R² can be pyridyl substituted with 1 R⁹.

Variable W

In some embodiments, W can be —O— or a bond.

In other embodiments, W can be —W¹(C₁₋₆ alkylene)-. In certain embodiments, W¹ can be —O—. For example, W can be —O(C₁₋₃ alkylene)- (e.g., —OCH₂—).

In some embodiments, W can be —NR¹⁰— (e.g., —NH—).

In some embodiments, W can be —(C₁₋₆ alkylene)W¹—. In certain embodiments, W¹ is —NH—; or W¹ can be —O—. In certain embodiments, W can be —(C₁₋₃ alkylene)NH— (e.g., —CH₂NH—). In certain embodiments, W can be —(C₁₋₃ alkylene)O— (e.g., —CH₂O—).

In still other embodiments, W can be C₂-C₄ alkenylene (e.g., —CH═CH—); C₂-C₄ alkynylene (e.g., —C≡C—); or C₁₋₃ alkylene (e.g., CH₂).

In certain embodiments, W is other than NR¹⁰. In certain embodiments, is other than —NH— or —N(C₁-C₆ alkyl).

Variable A

In general, A is an aromatic or heteroaromatic ring system that is (a) substituted with one R¹¹; and (b) optionally substituted with one or more R^{g}.

In some embodiments, A can be C₆-C₁₀ (e.g., phenyl) aryl, which is (a) substituted with 1 R¹¹; and (b) optionally further substituted with from 1-4 (e.g., 1-3, 1-2, 1, e.g., 1-2) R^{g}. In these and in the following embodiments related to variable A, R¹¹ and R^{g} can be as defined anywhere herein.

In some embodiments, A can be C₆-C₁₀ aryl, which is (i) substituted with 1 R¹¹ and (ii) optionally further substituted with from 1-2 R^{g}.

In embodiments, when A is aryl and substituted with one or more R^{g}, each R^{g} can be independently of one another:
halo (e.g., chloro or fluoro); or
C₁-C₆ (e.g., C₁-C₃) haloalkoxy; or
C₁-C₆ (e.g., C₁-C₃) alkoxy; NR^{m}Rⁿ; or
cyano; or
C₁-C₆ (e.g., C₁-C₃) alkyl or C₁-C₆ (e.g., C₁-C₃) haloalkyl.

In certain embodiments, R^{g} can be halo (e.g., chloro).

In some embodiments, A can be phenyl, which is (i) substituted with 1 R¹¹ and (ii) optionally substituted with from 1-4 (e.g., 1-3, 1-2, 1) R^{g}.

In some embodiments, A can be phenyl, which is (i) substituted with 1 R¹¹ and (ii) optionally substituted with from 1-2 R^{g}.

In these embodiments, R¹¹ can be attached to a ring carbon that is ortho, meta, or para (e.g., meta or para, e.g., meta) with respect to the ring carbon that connects the phenyl ring to W.

In certain embodiments, A can have formula (B-1):

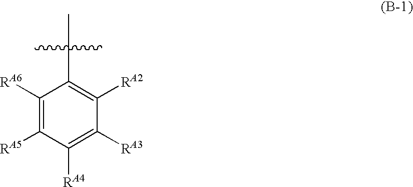

in which one of R^{A3} and R^{A4} is R¹¹, the other of R^{A3} and R^{A4} and each of R^{A2}, R^{A5}, and R^{A6} is, independently, hydrogen or R^{g}, in which R^{g} can be as defined anywhere herein.

In embodiments, one of R^{A3} and R^{A4} can be R¹¹, the other of R^{A3} and R^{A4} can be hydrogen; and each of R^{A2}, R^{A5}, and R^{A6} can be, independently, hydrogen or R^{g}.

In certain embodiments, R^{A3} can be R¹¹. For example, R^{A3} can be R¹¹, R^{A4} can be hydrogen, and each of R^{A2}, R^{A5}, and R^{A6} can be hydrogen. As another example, R^{A3} can be R¹¹; R^{A4} can be hydrogen; one of R^{A2}, R^{A5}, and R^{A6} (e.g., R^{A5}) can be R^{g} (e.g., halo) and the other two of R^{A2}, R^{A5}, and R^{A6} can be hydrogen.

In certain embodiments, R^{A4} can be R¹¹. For example, R^{A4} can be R¹¹, R^{A3} can be hydrogen, and each of R^{A2}, R^{A5}, and R^{A6} can be hydrogen. As another example, R^{A3} can be R¹¹; R^{A4} can be hydrogen; one of R^{A2}, R^{A5}, and R^{A6} can be R^{g} (e.g., halo) and the other two of R^{A2}, R^{A5}, and R^{A6} can be hydrogen.

In some embodiments, A can be heteroaryl including 5-10 atoms, which is (a) substituted 1 R¹¹; and (b) is optionally substituted with from 1-3 (e.g., 1-2, 1) R^{g}. In these and in the following embodiments related to variable, R¹¹ and R^{g} can be as defined anywhere herein.

In certain embodiments, A can be pyrrolyl, pyridyl, pyridyl-N-oxide, pyrazolyl, pyrimidinyl, thienyl, furyl, quinolinyl, oxazolyl, thiazolyl, imidazolyl, isoxazolyl, indolyl, benzo[1,3]-dioxolyl, benzo[1,2,5]-oxadiazolyl, isochromenyl-1-one, 3-H-isobenzofuranyl-1-one (e.g., pyridyl, thienyl, or indolyl, e.g., pyridyl), which is (i) substituted with 1 R¹¹ and (ii) optionally substituted with 1-3 (e.g., 1-2, 1) R^{g}.

In certain embodiments, A can be pyrrolyl, pyridyl, pyrimidinyl, pyrazolyl, thienyl, furyl, quinolyl, oxazolyl, thiazolyl, imidazolyl, or isoxazolyl, each of which is (a) substituted with 1 R¹¹; and (b) is optionally substituted with from 1-3 (e.g., 1-2, 1) R^{g}.

In certain embodiments, A can be pyridyl, pyrimidinyl, thienyl, furyl, oxazolyl, thiazolyl, imidazolyl, or isoxazolyl, each of which is (a) substituted with 1 R¹¹; and (b) is optionally substituted with from 1-3 (e.g., 1-2, 1) R^{g}.

In certain embodiments, A can be pyridyl in which W is attached to the 2- or 3-position of the pyridiyl ring. For example, A can be pyridyl in which W is attached to the 2-position of the pyridyl ring, and R¹¹ is attached to the 4- or the 6-position of the pyridyl ring. Such rings can be further substituted with 1, 2 or 3 R^{g} (e.g., halo, e.g., chloro; or NR^{m}Rⁿ, e.g., NH₂).

Variable R¹¹

R¹¹ can be:

(11-i) —W²—S(O)ₙR¹² or —W²—S(O)ₙNR¹³R¹⁴; or

(11-ii) —W²—C(O)OR¹⁵; or

(11-iii) —W²—C(O)NR¹³R¹⁴; or

(11-iv) C₁-C₁₂ alkyl or C₁-C₁₂ haloalkyl, each of which is:
(a) substituted with from 1 R^{h}, and
(b) optionally further substituted with from 1-5 R^{a};

or

(11-v) —NR¹⁶R¹⁷.

In some embodiments, R¹¹ can be:

(11-i′)—W²—S(O)ₙR¹²; or

(11-ii), (11-iii), (11-iv), or (11-v).

In some embodiments, R¹¹ can be any one of: (11-i), (11-i′), (11-ii), (11-iii), (11-iv), or (11-v). In certain embodiments, R¹¹ can be —W²—S(O)ₙR¹²or —W²—S(O)ₙNR¹³R¹⁴ (e.g., —W²—S(O)ₙR¹²). In other embodiments, R¹¹ can be —W²—C(O)OR¹⁵ or —W²—C(O)NR¹³R¹⁴; or NR¹⁶R¹⁷.

In some embodiments, R¹¹ can be any two of: (11-i), (11-i′), (11-ii), (11-iii), (11-iv), or (11-v). In certain embodiments, R¹¹ can be —W²—S(O)ₙR¹² or —W²—S(O)ₙNR¹³R¹⁴ (e.g., —W²—S(O)ₙR¹²) and any one of (11-ii), (11-iii), (11-iv), or (11-v). For example, R¹¹ can be:
—W²—S(O)ₙR¹² or —W²—S(O)ₙNR¹³R¹⁴ (e.g., —W²—S(O)ₙR¹²); and
—W²—C(O)OR¹⁵ or —W²—C(O)NR¹³R¹⁴; or NR¹⁶R¹⁷.

In other embodiments, R¹¹ can be any two of (11-ii), (11-iii), (11-iv), or (11-v).

In some embodiments, R¹¹ can be any three of: (11-i), (11-i′), (11-ii), (11-iii), (11-iv), or (11-v).

In certain embodiments, R¹¹ can be —W²—S(O)ₙR¹², —W²—S(O)ₙNR¹³R¹⁴, and —W²—C(O)OR¹⁵ or —W²—C(O)NR¹³R¹⁴; or NR¹⁶R¹⁷.

In certain embodiments, R¹¹ can be:
—W²—S(O)ₙR¹² or —W²—S(O)ₙNR¹³R¹⁴ (e.g., —W²—S(O)ₙR¹²); and
—W²—C(O)OR¹⁵; and
any one of (11-iii), (11-iv), or (11-v).

In other embodiments, R¹¹ can be (11-iii), (11-iv), or (11-v).

In some embodiments, W² can be a bond.

In some embodiments, R¹¹ can be —W²—S(O)ₙR¹² (e.g., —W²—S(O)ₙR¹², in which n is 2). In embodiments, W² can be a bond, i.e., R¹¹ is connected to variable A by the sulfur (S) atom of the sulfinyl or the sulfonyl group.

In some embodiments, R¹² can be C₁-C₆ (e.g., C₁-C₅) alkyl or C₁-C₆ (e.g., C₁-C₅ or C₁-C₃) haloalkyl, optionally substituted with from 1-2 R^{a}.

In certain embodiments, R¹² can be C₂-C₆ alkyl, that is substituted with from 1-2 (e.g., 1) R^{a}.

In certain embodiments, R¹² can be unsubstituted branched or unbranched C₁-C₆ (e.g., C₁-C₂, C₁-C₃, C₁-C₅, C₂-C₆, C₃, C₄, or C₃-C₆) alkyl. For example, R¹² can be methyl (CH₃). As another example, R¹² can be ethyl (CH₂CH₃). As a further example, R¹² can be isopropyl (CH(CH₃)₂).

In certain embodiments, R¹² can be branched or unbranched C₂-C₆ (e.g., C₃-C₆ or C₃-C₅) alkyl, which is substituted with 1 R^{a}. In embodiments, R^{a} can be: hydroxyl; C₁-C₆ (e.g., C₁-C₃) alkoxy; NR^{m}Rⁿ. For example, R^{a} can be hydroxyl, C₁-C₆ (e.g., C₁-C₃) alkoxy, or NR^{m}Rⁿ. In certain embodiments, R^{a} (e.g., hydroxyl) can be attached to a secondary or tertiary carbon atom of the alkyl group or a primary carbon of the alkyl group. In embodiments, R¹² can be hydroxyl substituted C₃-C₆ (e.g., C₃-C₅) alkyl. In other embodiments, R¹² can be C₃-C₆ (e.g., C₃-C₅) alkyl that is substituted with an amino group (NH₂) or a secondary or tertiary amino group.

In certain embodiments, R¹² can be branched or unbranched C₁-C₆ haloalkyl (e.g., having from 1-3, 1-2, or 1 halo).

In certain embodiments, R¹² can be C₇-C₁₁ aralkyl (e.g., benzyl), optionally substituted with from 1-3 (e.g., 1-2, 1) R^{c}.

In certain embodiments, R¹² can be C₆-C₁₀ aryl, optionally substituted with from 1-2 R^{d}.

In some embodiments, R¹¹ can be —W²—S(O)ₙNR¹³R¹⁴ (e.g., —W²—S(O)₂NR¹³R¹⁴, in which n is 2). In embodiments, W² can be a bond, i.e., R¹¹ is connected to variable A by the sulfur (S) atom of the sulfinamide or sulfonamide group.

In certain embodiments, one or both of R¹³ and R¹⁴ can be hydrogen. In certain embodiments, R¹¹ can be —S(O)₂NH₂.

In other embodiments, one of R¹³ and R¹⁴ can be hydrogen, and the other of R¹³ and R¹⁴ can be C₁-C₆ (e.g., C₁-C₃) alkyl optionally substituted with 1 R^{a}; C₃-C₇ cycloalkyl optionally substituted with 1 R^{c}; or heterocyclyl including 3-8 atoms or a heterocycloalkenyl including 3-10 atoms, each of which is optionally substituted with from 1-3 R^{c}.

In certain embodiments, R¹³ and R¹⁴ can each be, independently of one another: C₁-C₆ (e.g., C₁-C₃) alkyl optionally substituted with 1 R^{a}; C₃-C₇ cycloalkyl optionally substituted with 1 R^{c}; or heterocyclyl including 3-8 atoms or a heterocycloalkenyl including 3-10 atoms, each of which is optionally substituted with from 1-3 R^{c}.

In still other embodiments, R¹³ and R¹⁴ together with the nitrogen atom to which they are attached can form a heterocyclyl including 3-8 (e.g., 3-6) atoms or a heterocycloalkenyl including 3-8 (e.g., 3-6) atoms, each of which is optionally substituted with from 1-3 (1-2, 1) R^{c}. In some embodiments, the heterocyclyl can further include one or more additional ring heteroatoms (e.g., N, O, or S).

In certain embodiments, R¹³ and R¹⁴ together with the nitrogen atom to which they are attached can form a heterocyclyl including 3-8 (e.g., 3-6, or 5-6) atoms, which is optionally substituted with from 1-3 (1-2, 1) R^{c}. For example, R¹³ and R¹⁴ together with the nitrogen atom to which they are attached can form a morpholinyl, piperidyl, pyrrolidinyl, or piperazinyl ring, each of which is optionally substituted with from 1-3 (1-2, 1) R^{c}.

In some embodiments, R¹¹ can be —W²—C(O)OR¹⁵. In some embodiments, W² can be C₁-C₆ alkylene; or a bond. In certain embodiments, W² can be C₁-C₆ alkylene. For example, W² can be C₁-C₃ alkylene, such as CH₂ or CH₂CH₂. In other embodiments, W² can be a bond. In some embodiments, R¹⁵ can be: (i) hydrogen; or (ii) C₁-C₆ (e.g., C₁-C₃) alkyl.

In some embodiments, R¹¹ can be —W²—C(O)NR¹³R¹⁴

Embodiments can include, for example, any one or more of the features described above in conjunction with —W²—S(O)ₙNR¹³R¹⁴ and/or —W²—C(O)OR¹⁵.

In some embodiments, R¹¹ can be: C₁-C₆ alkyl or C₁-C₆ haloalkyl, each of which is (a) substituted with from 1 R^{h}, and (b) optionally further substituted with from 1 or 2 R^{a}; or

In certain embodiments, R^{h} at each occurrence can be, independently, hydroxyl, C₁-C₆ alkoxy, C₁-C₆ haloalkoxy; C₃-C₈ cycloalkoxy, which is optionally substituted with from 1-3 R^{c}; or C₆-C₁₀ aryloxy or heteroaryloxy including 5-10 atoms, each of which is optionally substituted with from 1-3 R^{d}.

In certain embodiments, R¹¹ can have the following formula: —C(R¹¹¹)(R¹¹²)(R^{h}), in which each of R¹¹¹ and R¹¹² is, independently, C₁-C₇ alkyl or C₁-C₇ haloalkyl, each of which is optionally further substituted with from 1 or 2 R^{a} (e.g., R^{a} can be C₃-C₇ cycloalkyl, which is optionally substituted with from 1-5 R^{c}); and R^{h} can be as defined anywhere herein.

In some embodiments, R¹¹ can be —NR¹⁶R¹⁷, one of R¹⁶ and R¹⁷ is hydrogen or C₁-C₃ alkyl (e.g., hydrogen); and the other of R¹⁶ and R¹⁷ can be:

(i) —S(O)ₙR¹²; or

(ii) —C(O)OR¹⁵; or

(iii) —C(O)NR¹³R¹⁴; or

(iv) C₁-C₁₂ alkyl or C₁-C₁₂ haloalkyl, each of which is:
(a) substituted with 1 R^{h}, and
(b) optionally further substituted with from 1-5 R^{a}.

In certain embodiments, one of R¹⁶ and R¹⁷ is hydrogen, and the other of R¹⁶ and R¹⁷ is —S(O)ₙR¹².

In embodiments, each of n, R¹², R¹³, R¹⁴, R¹⁵, R^{h}, R^{a}, and R^{d} can be, independently, as defined anywhere herein.

Variables R⁴ and R⁵

In some embodiments, each of R⁴ and R⁵ can be, independently:

(i) hydrogen; or

(ii) halo; or

(iii) C₁-C₆ alkyl or C₁-C₆ haloalkyl, each of which is optionally substituted with from 1-3 R^{a}; or

In certain embodiments, each of R⁴ and R⁵ can be, independently:

(i) hydrogen; or

(ii) halo; or

(iii) C₁-C₃ alkyl or C₁-C₃ haloalkyl (e.g., perhaloalkyl, e.g., perfluoroalkyl), each of which is optionally substituted with from 1-3 R^{a}.

In certain embodiments, each of R⁴ and R⁵ can be, independently, hydrogen or halo (e.g., fluoro).

In certain embodiments, each of R⁴ and R⁵ can be hydrogen.

In certain embodiments, each of R⁴ and R⁵ can be a substituent other than hydrogen (e.g., halo, e.g., fluoro).

Variable R³ and R⁶

In some embodiments, one of R³ and R⁶ can be:

(i) halo; or

(ii) C₁-C₆ alkyl or C₁-C₆ haloalkyl, each of which is optionally substituted with from 1-3 R^{a} (e.g., C₁-C₆ haloalkyl, each of which is optionally substituted with from 1-3 R^{a}); or

(iii) nitro; hydroxy; C₁-C₆ alkoxy; C₁-C₆ haloalkoxy; C₁-C₆ thioalkoxy; C₁-C₆ thiohaloalkoxy; cyano; or S(O)_{z}(C₁-C₃ alkyl), wherein z is 1 or 2;

and the other of R³ and R⁶ can be:

(i) hydrogen; or

(ii) halo; or

(iii) C₁-C₆ alkyl or C₁-C₆ haloalkyl, each of which is optionally substituted with from 1-3 R^{a} (e.g., C₁-C₆ haloalkyl, each of which is optionally substituted with from 1-3 R^{a}); or

(iv) nitro; hydroxy; C₁-C₆ alkoxy; C₁-C₆ haloalkoxy; C₁-C₆ thioalkoxy; C₁-C₆ thiohaloalkoxy; cyano; or S(O)_{z}(C₁-C₃ alkyl), wherein z is 1 or 2.

In certain embodiments, one of R³ and R⁶ can be:

(i) halo; or

(ii) C₁-C₆ haloalkyl; or

(iii) C₁-C₆ alkoxy; C₁-C₆ haloalkoxy; or; cyano;

and the other of R³ and R⁶ can be:

(i) hydrogen; or

(ii) halo; or

(iii) C₁-C₆ haloalkyl; or

(iv) C₁-C₆ alkoxy; C₁-C₆ haloalkoxy; or cyano.

In some embodiments, one of R³ and R⁶ can be:

(i) halo; or

(ii) C₁-C₆ alkyl or C₁-C₆ haloalkyl, each of which is optionally substituted with from 1-3 R^{a}; or

(iii) nitro; hydroxy; C₁-C₆ alkoxy; C₁-C₆ haloalkoxy; C₁-C₆ thioalkoxy; C₁-C₆ thiohaloalkoxy; cyano; or S(O)_{z}(C₁-C₃ alkyl), wherein z is 1 or 2;

and the other of R³ and R⁶ can be hydrogen.

In certain embodiments, one of R³ and R⁶ can be:

(i) halo; or

(ii) C₁-C₆ haloalkyl; or

(iii) C₁-C₆ alkoxy; C₁-C₆ haloalkoxy; or cyano;

and the other of R³ and R⁶ can be hydrogen.

In certain embodiments, one of R³ and R⁶ can be halo (e.g., chloro), the other of R³ and R⁶ can be hydrogen.

In certain embodiments, one of R³ and R⁶ can be C₁-C₄ haloalkyl (e.g., C₁-C₄ perfluoroalkyl, CF₃), the other of R³ and R⁶ is hydrogen.

In certain embodiments, R³ can be hydrogen, and R⁶ can be CF₃.

In certain embodiments, R³ can be CF₃, and R⁶ can be hydrogen.

In some embodiments, each of R³ and R⁶ can be, independently:

(i) halo; or

(ii) C₁-C₆ alkyl or C₁-C₆ haloalkyl, each of which is optionally substituted with from 1-3 R^{a} (e.g., C₁-C₆ haloalkyl, each of which is optionally substituted with from 1-3 R^{a}); or

(iii) nitro; hydroxy; C₁-C₆ alkoxy; C₁-C₆ haloalkoxy; C₁-C₆ thioalkoxy; C₁-C₆ thiohaloalkoxy; cyano; or S(O)_{z}(C₁-C₃ alkyl), wherein z is 1 or 2.

In certain embodiments, each of R³ and R⁶ can be, independently:

(i) halo; or

(ii) C₁-C₆ haloalkyl; or

(iii) C₁-C₆ alkoxy; C₁-C₆ haloalkoxy; or cyano;

For example, each of R³ and R⁶ can be, independently, halo (e.g., chloro).

In some embodiments, each of R³ and R⁶ can be hydrogen.

A subset of compounds includes those in which R² has formula (A-2):

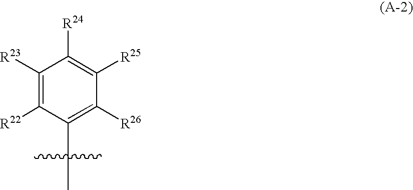

in which:

one of R²³ and R²⁴ can be R⁹ (e.g., one of R²³ and R²⁴ can have formula (C-1):

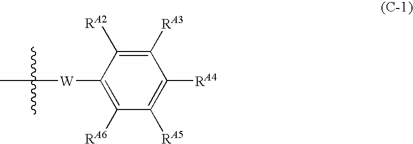

in which one of R^{A2}, R^{A3}, R^{A4}, R^{A5}, and R^{A6} is R¹¹, and the others are each, independently, hydrogen or R^{g});

and the other of R²³ and R²⁴ is hydrogen;

and each of R²², R²⁵, and R²⁶ is, independently, hydrogen or R^{e}.

In these and in the following embodiments, R²², R²³, R²⁴, R²⁵, R²⁶, W, R^{A2}, R^{A3}, R^{A4}, R^{A5}, R^{A6}, R⁹, R¹¹, R^{e}, and R^{g} can be, independently, as defined anywhere herein. By way of example, the compounds can include one or more of the following features.

R²³ can have formula (C-1), and R²⁴ can be hydrogen.

R²³ can be hydrogen, and R²⁴ can have formula (C-1).

Each of R²², R²⁵, and R²⁶ can be hydrogen.

One of R²², R²⁵, and R²⁶ can be R^{e}, and the other two can each be hydrogen. For example, R²⁶ can be R^{e}, and each of R²² and R²⁵ can be hydrogen. R^{e} can be: halo (e.g., chloro or fluoro); C₁-C₃ alkyl; or C₁-C₃ haloalkyl (e.g., C₁-C₃ fluoroalkyl, e.g., 1-5 fluorines can be present; or C₁-C₃ perfluoroalkyl). In certain embodiments, R^{e} can be halo (e.g., chloro or fluoro, e.g., chloro).

W can be —O—.

W can be a bond.

One of R^{A3} and R^{A4} (e.g., R^{A3}) can be R¹¹, and the other of R^{A3} and R^{A4} (e.g., R^{A4}) can be hydrogen; and each of R^{A2}, R^{A5}, and R^{A6} is, independently, hydrogen or R^{g}.

R¹¹ can be —W²—S(O)ₙR¹². W² can be a bond. n can be 2. R¹² can be C₁-C₆ alkyl, optionally substituted with from 1-2 R^{a}. For example, R¹² can be C₁-C₃ alkyl (e.g., CH₃). As another example, R¹² can be C₁-C₆ alkyl substituted with 1 R^{a}, in which R^{a} is hydroxyl or NR^{m}Rⁿ.

Each of R^{A2}, R^{A5}, and R^{A6} can be hydrogen.

R^{A5} can be hydrogen or R^{g}, and each of R^{A2} and R^{A6} is hydrogen. R^{A5} can be R^{g} (e.g., halo).

R^{A3} can be R¹¹, R^{A4} can be hydrogen, and each of R^{A2}, R^{A5}, and R^{A6} can be hydrogen; or R^{A3} can be R¹¹; R^{A4} can be hydrogen; one of R^{A2}, R^{A5}, and R^{A6} (e.g., R^{A5}) can be R^{g} (e.g., halo, e.g., fluoro) and the other two of R^{A2}, R^{A5}, and R^{A6} can be hydrogen.

R^{A4} can be R¹¹, R^{A3} can be hydrogen, and each of R^{A2}, R^{A5}, and R^{A6} can be hydrogen. R^{A3} can be R¹¹; R^{A4} can be hydrogen; one of R^{A2}, R^{A5}, and R^{A6} can be R^{g} (e.g., halo) and the other two of R^{A2}, R^{A5}, and R^{A6} can be hydrogen.

R¹¹ can be —W²—S(O)ₙNR¹³R¹⁴. W² can be a bond, and one of R¹³ and R¹⁴ can be C₁-C₃ alkyl, and the other of R¹³ and R¹⁴ can be hydrogen.

Other embodiments can include one of more other features described herein and present in combination with the features delineated above.

In some embodiments, the compounds can have formula (II):

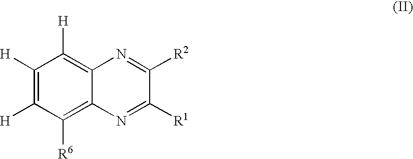

in which each of R¹ and R² can be, independently, as defined anywhere herein (generically, subgenerically, or specifically), and R⁶ is a substituent other than hydrogen (e.g., halo, e.g., chloro; or C₁-C₄ haloalkyl, e.g., C₁-C₄ perfluoroalkyl, e.g., CF₃).

In some embodiments, the compounds can have formula (III):

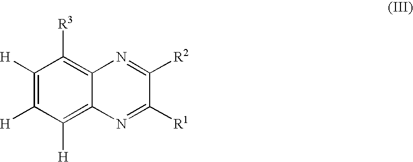

in which each of R¹ and R² can be, independently, as defined anywhere herein (generically, subgenerically, or specifically), and R³ is a substituent other than hydrogen (e.g., halo, e.g., chloro; or C₁-C₄ haloalkyl, e.g., C₁-C₄ perfluoroalkyl, e.g., CF₃).

In some embodiments, the compounds can have formula (IV):

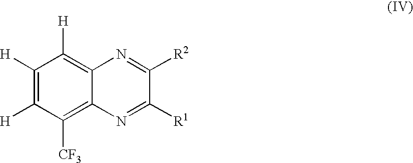

in which each of R¹ and R² can be, independently, as defined anywhere herein (generically, subgenerically, or specifically).

In some embodiments, the compounds can have formula (VI):

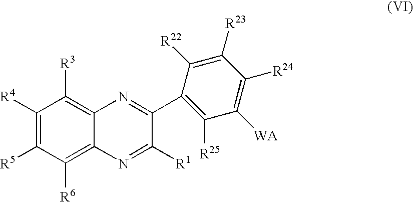

in which each of R¹, R³, R⁴, R⁵, R⁶, R²², R²³, R²⁴, W, and A can be, independently, as defined anywhere herein (generically, subgenerically, or specifically).

In embodiments, the compounds of formulas (II), (III), (IV), and (VI) can include any one or more of the following features.

R¹ can be (i) hydrogen; or (ii) C₁-C₃ alkyl or C₁-C₃ haloalkyl; or (iii) NR⁷R⁸. For example, R¹ can be C₁-C₃ alkyl (e.g., CH₃).

One of R³ and R⁶ can be (i) halo; or (ii) C₁-C₄ haloalkyl; or (iii) C₁-C₆ alkoxy; C₁-C₆ haloalkoxy; or cyano; and the other of R³ and R⁶ is hydrogen. For example, one of R³ and R⁶ can be halo, e.g., chloro; or C₁-C₄ haloalkyl, e.g., C₁-C₄ perfluoroalkyl, e.g., CF₃; and the other of R³ and R⁶ can be hydrogen.

Each of R⁴ and R⁵ can be hydrogen.

R² can have formula (A), (A-1), (A-2), or (C-1) as defined anywhere herein.

W can be —O—.

W can be a bond.

In some embodiments, A can be phenyl, which is (i) substituted with 1 R¹¹ and (ii) optionally substituted with from 1-4 (e.g., 1-3, 1-2, 1) R^{g}, in which R^{g} can be as defined anywhere herein.

A can have formula (B-1). In embodiments, one of R^{A3} and R^{A4} is R¹¹, and the other of R^{A3} and R^{A4} is hydrogen; and each of R^{A2}, R^{A5}, and R^{A6} is, independently, hydrogen or R^{g}, in which R¹¹ and R^{g} can be as defined anywhere herein.

Each of R^{e}, R¹¹, and R^{g} can be, independently, as defined anywhere herein.

R¹¹ can be —W²—S(O)ₙR¹² or —W²—S(O)ₙNR¹³R¹⁴ (e.g., —W²—S(O)ₙR¹²).

Each of R¹², R¹³, R¹⁴, and R¹⁵ can be, independently, as defined anywhere herein (e.g., as defined in conjunction with formula (C-1)).

W², n, R²², R²³, R²⁴, R^{A2}, R^{A3}, R^{A4}, R^{A5}, and R^{A6} can be as defined in conjunction with formula (C-1).

It is understood that the actual electronic structure of some chemical entities cannot be adequately represented by only one canonical form (i.e. Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art recognizes as the predominant resonance form for a particular species.

The compounds described herein can be synthesized according to methods described herein (or variations thereof) and/or conventional, organic chemical synthesis methods from commercially available starting materials and reagents or from starting materials and reagents that can be prepared according to conventional organic chemical synthesis methods. The compounds described herein can be separated from a reaction mixture and further purified by a method such as column chromatography, high-performance liquid chromatography (HPLC), or recrystallization. As can be appreciated by the skilled artisan, further methods of synthesizing the compounds of the formulae herein will be evident to those skilled in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. C. Larock, Comprehensive Organic Transformations, 2d. ed., Wiley-VCH Publishers (1999); P. G. M. Wuts and T. W. Greene, Protective Groups in Organic Synthesis, 4th Ed., John Wiley and Sons (2007); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this invention can be readily prepared according to the following schemes from commercially available starting materials or starting materials which can be prepared using literature procedures. The schemes show the preparation of representative compounds of this invention. It is also possible to make use of variants of these process steps, which in themselves are known to and well within the preparatory skill of the skilled artisan. In the following reaction schemes, R¹ to R⁶, and A are selected from groups defined above.

According to Scheme 1, quinoxalines can be prepared by condensation of 1,2-dioxoalkanes 1 with 1,2-diaminobenzenes 2, typically in a solvent such as ethanol at temperatures, typically 0 to 120° C. These reaction conditions typically provide a mixture of regioisomers 3 and 4 which can be separated by a method such as column chromatography, high-performance liquid chromatography (HPLC), or recrystallization. The structure of 3 and 4 can be assigned by NMR techniques such as ¹H-¹³C HMBC, ¹H-¹H nOe. In compounds 3 and 4, in which T is a protected hydroxyl group such as a methoxy or benzyloxy, deprotection of the hydroxyl group leads to compounds 5 and 6, respectively. Typical conditions for deprotection when T is a methoxy include treatment with HBr or treatment with BBr₃ at elevated temperatures, typically 60-150° C., for 0.1 to 24 h, or other methods known to those skilled in the art. Phenols 5 and 6 can be converted to triflates 7 and 8, respectively, using triflic anhydride or N-phenylbis(trifluoromethanesulfonamide) in the presence of a base such as triethylamine or potassium carbonate. The resulting triflates 7 and 8 can be coupled to an aryl boronic acid or ester under catalysis with a palladium catalyst, a reaction known as a Suzuki reaction to those skilled in the art, to give the biaryl derivatives 9 and 10, respectively.

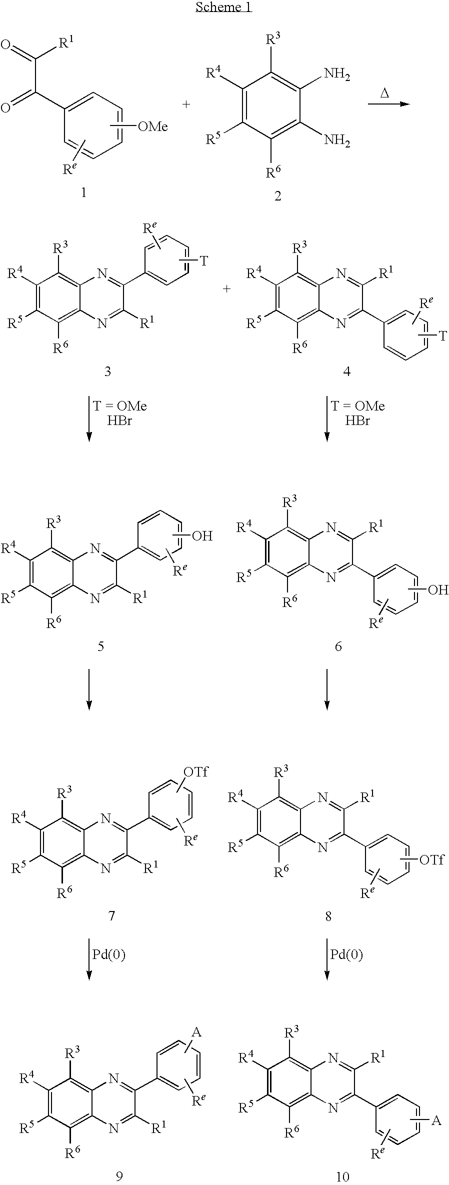

Alternatively, according to Scheme 2, the phenol 5 and 6 can be treated with a halogenated aromatic ring-containing compound X-A (where X is a halogen) to provide biarylether 11 and 12, respectively. If the halogen is a fluorine or chlorine atom, the formation of the biarylether can be accomplished by treatment with a base such as potassium carbonate, typically in a polar solvent such as dimethylformamide or dimethylsulfoxide, at elevated temperatures, typically 100° C. to 150° C. for 1 to 48 hours. Alternatively, where the halogen is a bromine or iodine, the formation of the biarylether can be accomplished with a coupling reaction using a metal catalyst such as a copper salt or a palladium salt in the presence of a base and a solvent such as dioxane at elevated temperatures.

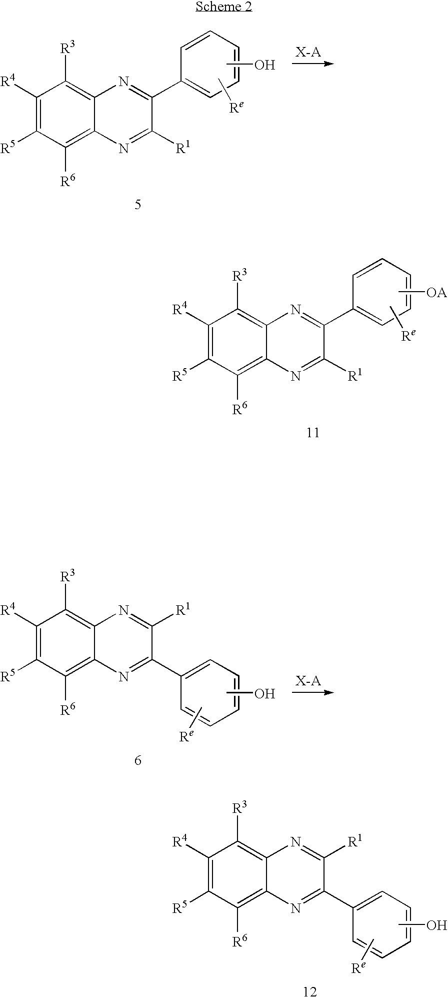

Alternatively, according to Scheme 3, quinoxalines can be prepared by condensation of biaryl 1,2-dioxoalkanes such as 13 with 1,2-diaminobenzenes 2, typically in a solvent such as ethanol at temperatures, typically 0 to 120° C.

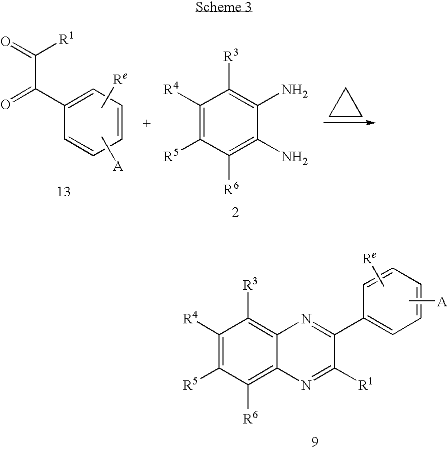

Alternatively, according to Scheme 4, quinoxalines can be prepared by amination of aldehydes such as 14 with 1,2-diaminobenzenes 2, typically in a solvent such as ethanol at temperatures, typically 0 to 120° C. Treatment of the resulting imine 15 with a reagent such as potassium cyanide in a solvent like methanol provides amino quinoxalines 16. This can then be coupled to an aryl boronic acid or ester under catalysis with a palladium catalyst, a reaction known as a Suzuki reaction to those skilled in the art, to give the biaryl derivatives 17.

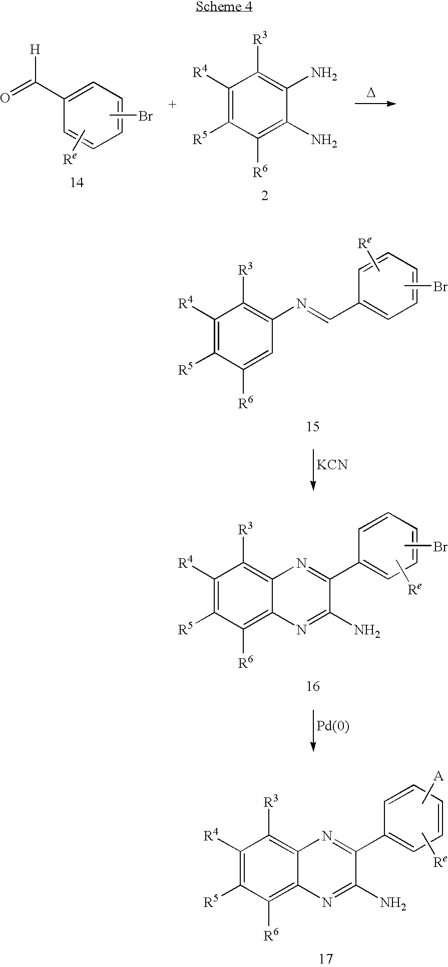

According to Scheme 5 condensation of diamine 2 with bis-carbonyls such as pyruvates or glyoxalates gives quinoxalinones such as 18, along with the undesired isomer. 18 can be treated with chlorinating agents under standard conditions to provide the chloro-quinoxalines 19. Under basic conditions these can be treated with phenols to provide quinoxaline ethers such as 20 which can be substituted using an aryl boronic acid or ester under catalysis with a palladium catalyst to provide 21. Quinoxaline amines (analogs of 21 in which the —O— linkage is replaced with —NH—) can be made by analogous procedures using anilines in place of phenols.

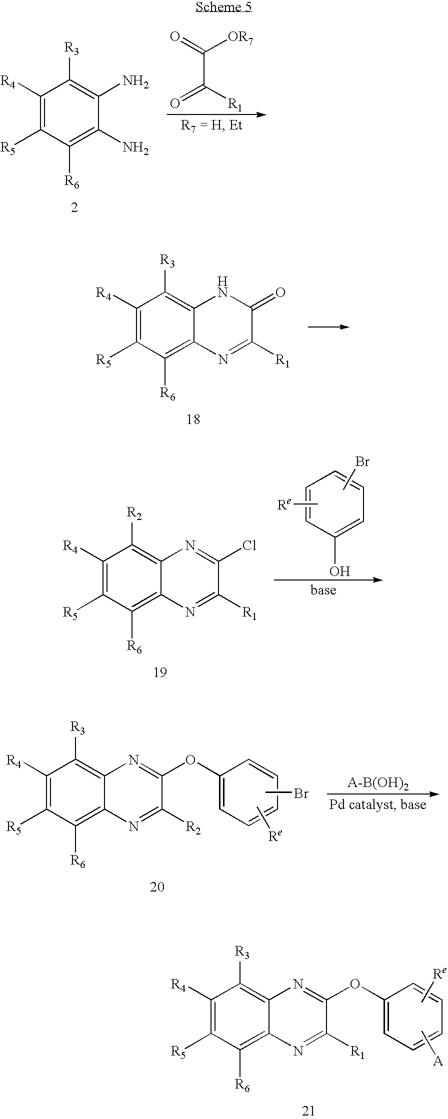

The compounds of this invention may contain one or more asymmetric centers and thus occur as racemates and racemic mixtures, enantiomerically enriched mixtures, single enantiomers, individual diastereomers and diastereomeric mixtures. All such isomeric forms of these compounds are expressly included in the present invention. The compounds of this invention may also contain linkages (e.g., carbon-carbon bonds, carbon-nitrogen bonds such as amide bonds) wherein bond rotation is restricted about that particular linkage, e.g. restriction resulting from the presence of a ring or double bond. Accordingly, all cis/trans and E/Z isomers and rotational isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include C₁₋₆ alkyl esters of carboxylic acid groups, which, upon administration to a subject, are capable of providing active compounds.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

The term “pharmaceutically acceptable carrier or adjuvant” refers to a carrier or adjuvant that may be administered to a subject (e.g., a patient), together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts, or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α, β, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

In general, the compounds described herein can be used for, treating (e.g., controlling, relieving, ameliorating, alleviating, slowing the progression of, delaying the onset of, or reducing the risk of developing) or preventing one or more diseases, disorders, conditions or symptoms mediated by LXRs (e.g., cardiovascular diseases (e.g., acute coronary syndrome, restenosis, or coronary artery disease), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging (e.g., skin aging is derived from chronological aging, photoaging, steroid-induced skin thinning, or a combination thereof), or connective tissue disease (e.g., osteoarthritis or tendonitis).

A disorder or physiological condition that is mediated by LXR refers to a disorder or condition wherein LXR can trigger the onset of the condition, or where inhibition of a particular LXR can affect signaling in such a way so as to treat, control, ameliorate, alleviate, prevent, delay the onset of, slow the progression of, or reduce the risk of developing the disorder or condition. Examples of such disorders include, but are not limited to cardiovascular diseases (e.g., acute coronary syndrome, restenosis, or coronary artery disease), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging (e.g., skin aging is derived from chronological aging, photoaging, steroid-induced skin thinning, or a combination thereof), or connective tissue disease (e.g., osteoarthritis or tendonitis).

While not wishing to be bound by theory, it is believed that LXR modulators that activate cholesterol efflux (e.g., upregulate ABCA1), but do not substantially increase SREBP-1c expression and triglyceride synthesis in liver, can both reduce atherosclerotic risk and minimize the likelihood of concommitantly increasing serum and hepatic triglyceride levels. Candidate compounds having differential activity for regulating ABCA1 (ABCG1) vs. SREBP-1c can be can be evaluated using conventional pharmacological test procedures, which measure the affinity of a candidate compound to bind to LXR and to upregulate the gene ABCA1.

In some embodiments, LXR ligands can be identified initially in cell-free LXR beta and LXR alpha competition binding assays. LXR ligands can be further characterized by gene expression profiling for tissue selective gene regulation.

In some embodiments, the compounds described herein have agonist activity for ABCA1 transactivation but do not substantially affect (e.g., inhibit) SREBP-1c gene expression in differentiated THP-1 macrophages. Gene expression analysis in an antagonist mode can be used to further delineate differential regulation of ABCA1 and SREBP-1c gene expression. In certain embodiments, the compounds described herein preferentially antagonize SREBP-1c activation (a marker for genes involved in cholesterol and fatty acid homeostasis) but do not substantially affect (e.g., have relatively minimal or additive effects) on ABCA1 gene expression or genes known to enhance HDL biogenesis (based on a competition assay with known potent synthetic LXR agonists). Cell type or tissue specificity may be further evaluated in additional cell lines, intestinal, CaCo2 or liver, HepG2 and Huh-7 cells where ABCA1 activity is believed to influence net cholesterol absorption and reverse cholesterol transport. The test procedures performed, and results obtained therefrom are described in the Examples section.

In some embodiments, the compounds described herein have agonist activity for ABCA1 and antagonist activity for SREBP-1c (e.g., as determined by gene specific modulation in cell based assays). In certain embodiments, the compounds described herein (in the agonist mode) have at least about 20% efficacy for ABCA1 activation by LXR and do not substantially agonize SREBP-1c (at most about 25% efficacy relative to a reference compound N-(2,2,2-trifluoro-ethyl)-N-[4-(2,2,2-trifluoro-1-hydroxy-1-trifluoromethyl-ethyl)-phenyl]-benzenesulfonamide (Schultz, Joshua R., Genes & Development (2000), 14(22), 2831-2838)). In certain embodiments, the compounds described herein (in the antagonist mode) do not substantially antagonize ABCA1 gene expression. While not wishing to be bound by theory, it is believed that there may be an additive effect on ABCA1 gene expression relative to the reference compound at their EC₅₀ concentration. In certain embodiments, the compounds described herein (in the antagonist mode) inhibited agonist-mediated SREBP-1c gene expression in a dose dependent fashion.

In some embodiments, to study the effect of the compounds of formula (I) on skin aging, for example, in a clinical trial, cells can be isolated and RNA prepared and analyzed for the levels of expression of TIMP1, ABCA12, decorin, TNFα, MMP1, MMP3, and/or IL-8. The levels of gene expression (i.e., a gene expression pattern) can be quantified, for example, by Northern blot analysis or RT-PCR, by measuring the amount of protein produced, or by measuring the levels of activity of TIMP1, ABCA12, decorin, TNFα, MMP1, MMP3, and/or IL-8, all by methods known to those of ordinary skill in the art. In this way, the gene expression pattern can serve as a marker, indicative of the physiological response of the cells to the compounds of formula (I). Accordingly, this response state may be determined before, and at various points during, treatment of the individual with the compounds of formula (I).

In one embodiment, expression levels of cytokines and metalloproteases described herein can be used to facilitate design and/or identification of compounds that treat skin aging through an LXR-based mechanism. Accordingly, the invention provides methods (also referred to herein as “screening assays”) for identifying modulators, i.e., LXR modulators, that have a stimulatory or inhibitory effect on, for example, TIMP1, ABCA12, decorin, TNFα, MMP1, MMP3, and/or IL-8 expression.

An exemplary screening assay is a cell-based assay in which a cell that expresses LXR is contacted with a test compound, and the ability of the test compound to modulate TIMP1, ABCA12, decorin, TNFα, MMP1, MMP3, and/or IL-8 expression through an LXR-based mechanism. Determining the ability of the test compound to modulate TIMP1, ABCA12, decorin, TNFα, MMP1, MMP3, and/or IL-8 expression can be accomplished by monitoring, for example, DNA, mRNA, or protein levels, or by measuring the levels of activity of TIMP1, ABCA12, decorin, TNFα, MMP1, MMP3, and/or IL-8, all by methods known to those of ordinary skill in the art. The cell, for example, can be of mammalian origin, e.g., human.

In some embodiments, to study the effect of the compounds of formula (I) on osteoarthritis, for example, in a clinical trial, cells can be isolated and RNA prepared and analyzed for the levels of expression of ApoD and other genes implicated in osteoarthritis (for example, TNFα). The levels of gene expression (i.e., a gene expression pattern) can be quantified by Northern blot analysis or RT-PCR, by measuring the amount of protein produced, or by measuring the levels of activity of ApoD or other genes, all by methods known to those of ordinary skill in the art. In this way, the gene expression pattern can serve as a marker, indicative of the physiological response of the cells to the LXR modulator. Accordingly, this response state may be determined before, and at various points during, treatment of the individual with the LXR modulator.

An exemplary screening assay is a cell-based assay in which a cell that expresses LXR is contacted with a test compound, and the ability of the test compound to modulate ApoD expression and/or aggrecanase activity and/or cytokine elaboration through an LXR-based mechanism. Determining the ability of the test compound to modulate ApoD expression and/or aggrecanase activity and/or cytokine elaboration can be accomplished by monitoring, for example, DNA, mRNA, or protein levels, or by measuring the levels of activity of ApoD, aggrecanase, and/or TNFα, all by methods known to those of ordinary skill in the art. The cell, for example, can be of mammalian origin, e.g., human.

In some embodiments, the compounds described herein can be coadministered with one or more other therapeutic agents. In certain embodiments, the additional agents may be administered separately, as part of a multiple dose regimen, from the compounds of this invention (e.g., sequentially, e.g., on different overlapping schedules with the administration of one or more compounds of formula (I) (including any subgenera or specific compounds thereof)). In other embodiments, these agents may be part of a single dosage form, mixed together with the compounds of this invention in a single composition. In still another embodiment, these agents can be given as a separate dose that is administered at about the same time that one or more compounds of formula (I) (including any subgenera or specific compounds thereof) are administered (e.g., simultaneously with the administration of one or more compounds of formula (I) (including any subgenera or specific compounds thereof)). When the compositions of this invention include a combination of a compound of the formulae described herein and one or more additional therapeutic or prophylactic agents, both the compound and the additional agent can be present at dosage levels of between about 1 to 100%, and more preferably between about 5 to 95% of the dosage normally administered in a monotherapy regimen.

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/kg to about 1000 mg/kg, (e.g., from about 0.01 to about 100 mg/kg, from about 0.1 to about 100 mg/kg, from about 1 to about 100 mg/kg, from about 1 to about 10 mg/kg) every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, N.Y., 537 (1970). In certain embodiments, the compositions are administered by oral administration or administration by injection. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compositions of this invention is useful when the desired treatment involves areas or organs readily accessible by topical application. For application topically to the skin, the composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions of this invention may also be topically applied to the lower intestinal tract by rectal suppository formulation or in a suitable enema formulation.

In some embodiments, topical administration of the compounds and compositions described herein may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman and Kanig, published by Lea and Febiger (1970) and in Chapter 67 of Remington's Pharmaceutical Sciences, 21st Edition (2005) published by Mack Publishing Company, which is incorporated herein by reference in its entirety.

Topically-transdermal patches are also included in this invention. Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

The compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using any of the routes of administration described herein. In some embodiments, a composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

The invention will be further described in the following examples. It should be understood that these examples are for illustrative purposes only and are not to be construed as limiting this invention in any manner.

### EXAMPLES

The following describes the preparation of representative compounds of this invention. Compounds described as homogeneous are determined to be of 90% or greater purity (exclusive of enantiomers) by analytical reverse phase chromatographic analysis with 254 nM UV detection. Melting points are reported as uncorrected in degrees centigrade. Mass spectral data is reported as the mass-to-charge ratio, m/z; and for high resolution mass spectral data, the calculated and experimentally found masses, [M+H]⁺, for the neutral formulae M are reported. All reactions are stirred and run under a nitrogen atmosphere unless otherwise noted. Eluents for chromatography are indicated by E for ethyl acetate and H for hexanes. Thus, for example, the expression “30:70 E:H” refers to a mixture of 30% ethyl acetate and 70% hexanes by volume.

### Example 1

### 3-methyl-2-[3′-(methylsulfonyl)biphenyl-4-yl]-5-(trifluoromethyl)quinoxaline

Step 1: A mixture of 2-nitro-3-(trifluoromethyl)aniline (2.6 g, 12.6 mmol), ethanol (20 mL) and 10% Pd/C (1.0 g) was pressurized with 25 psi H₂ for 1.5 hours. The catalyst was then removed by filtering through a short pad of celite. The filtrate was used for the next reaction with out any purification.

Step 2: 1-(4-Methoxyphenyl)propane-1,2-dione (2.5 g, 14.0 mmol) was added to the solution of 3-(trifluoromethyl)benzene-1,2-diamine in ethanol which was obtained from Step 1. The mixture was stirred at room temperature for 1 hour and the solvent was removed. The residue was purified by flash chromatography eluted with EtOAc/hexane to give 2-(4-methoxyphenyl)-3-methyl-5-(trifluoromethyl)quinoxaline as a pale yellow solid (1.4 g, 35% for two steps); MS (ES) m/z 319.1. The regio isomer 3-(4-methoxyphenyl)-2-methyl-5-(trifluoromethyl)quinoxaline (2.0 g, 50%) was also isolated from the reaction mixture.

Step 3: A mixture of 2-(4-methoxyphenyl)-3-methyl-5-(trifluoromethyl)quinoxaline (1.4 g, 4.39 mmol), HBr (48% in water, 20 mL) in 20 mL of acetic acid was heated to 90° C. over night. 7 mL of HBr (45%) in acetic acid was added and the reaction mixture was heated to reflux for 5 hours. The reaction mixture was poured into ice, extracted with EtOAc. The organic was concentrated and purified by flash chromatography eluted with EtOAc/hexane to give 4-[3-methyl-5-(trifluoromethyl)quinoxalin-2-yl]phenol (1.20 g, 90%) as a gummy solid; MS (ESI) m/z 305.1; HRMS: calcd for C₁₆H₁₁F₃N₂O+H+, 305.08962. found (ESI, [M+H]+Obs'd), 305.0894.

Step 4: A mixture of 4-[3-methyl-5-(trifluoromethyl)quinoxalin-2-yl]phenol (1.20 g, 3.93 mmol), anhydrous THF (40 mL), and N-phenylbis(trifluoromethanesulphonimide) (2.11 g, 5.93 mmol) was cooled to 0° C. upon which potassium tert-butoxide (0.62, 5.53 mmol) was added. The resulting mixture was stirred at 0° C. for 1 hour. Another portion of N-phenylbis(trifluoromethanesulphonimide) (2.11 g, 5.93 mmol) and potassium tert-butoxide (0.62, 5.53 mmol) was added. After 1 more hour the reaction was quenched with water, partitioned between water and EtOAc, and the organic was dried over MgSO₄. The residue was subjected to flash silica gel chromatography (hexane:EtOAc) to afford 4-[3-methyl-5-(trifluoromethyl)quinoxalin-2-yl]phenyl trifluoromethanesulfonate (1.2 g, 65%) as a colored solid; MS (ES) m/z 436.9.

Step 5: A mixture of 4-[3-methyl-5-(trifluoromethyl)quinoxalin-2-yl]phenyl trifluoromethanesulfonate (1.2 g, 2.75 mmol), 3-methylsulfonylphenyl boronic acid (2.4 g, 12 mmol), K₃PO₄ (5.0 g, 23.6 mmol), Pd(PPh₃)₄ (0.5 g, 0.43 mmol) in 40 mL of dioxane was heated to 80° C. for 1 hour. The reaction mixture was poured into water, extracted with EtOAc. The organic was concentrated and purified by flash chromatography eluted with EtOAc/hexane to give the title compound (0.59 g, 48%) as a white solid; MS (ES) m/z 443.0; HRMS: calcd for C₂₃H₁₇F₃N₂O₂S+H+, 443.10356. found (ESI, [M+H]+Obs'd), 443.1040.

### Example 2

### 3-methyl-2-{3-[3-(methylsulfonyl)phenoxy]phenyl}-5-(trifluoromethyl)quinoxaline

Step 1: In a reaction flask equipped with a magnetic stir bar under CaSO₄ tube was placed 3-(trifluoromethyl)benzene-1,2-diamine (750 mg, 4.261 mmol) and 1-(3-methoxyphenyl)propane-1,2-dione (910 mg, 5.114 mmol) in 2-propanol (50 ml). The resulting dark red solution was heated at reflux for 1.5 h. Cooling to room temperature upon which the dark red solution was concentrated in vacuo to a dark red powder. To this same power purification by SiO₂ chromatography (Hex:EtOAc) and concentration in vacuo of the cleanest fractions afforded two regio isomers. The second isolated product being 2-(3-methoxyphenyl)-3-methyl-5-(trifluoromethyl)quinoxaline as a red powder (0.512 g, 38% yield). MS (ES) m/z 319.0.

Step 2: In a reaction vial equipped with a magnetic stir bar was placed 2-(3-methoxyphenyl)-3-methyl-5-(trifluoromethyl)quinoxaline (400 mg, 1.258 mmol) and Hydrobromic acid 48% in HOAc (10 ml). The vial was tightly capped and heated at 90° C. for 2 h. Cooling to room temperature upon which pouring into H₂O (20 ml) and EtOAc (10 ml). Neutralization with solid NaHCO₃ until pH<7. Extraction, separation and extraction of the aqueous layer with EtOAc (20 ml). All organics combined, dried MgSO₄, filtration concentration in vacuo to a red powder. To this same power purification by SiO₂ chromatography (Hex:EtOAc) and concentration in vacuo of the cleanest fractions afforded 3-[3-methyl-5-(trifluoromethyl)quinoxalin-2-yl]phenol as a red powder (346 mg, 90% yield). MS (ES) m/z 304.7; HRMS: calcd for C₁₆H₁₁F₃N₂O+H+, 305.08962. found (ESI, [M+H]⁺ Obs'd), 305.0899.

Step 3: To a reaction vial suitable for microwave reactions containing a magnetic stir bar was placed 3-(3-methyl-5-(trifluoromethyl)quinoxalin-2-yl)phenol (50 mg, 0.164 mmol), 1-fluoro-3-(methylsulfonyl)benzene (57 mg, 0.328 mmol) and K₂CO₃ (68 mg, 0.493 mmol) in DMA (4 ml). The vial was capped put in a microwave reactor (Personal Chemistry unit) and the sample was irradiated at 180° C. for 1 h. Cooling to room temperature and partition between EtOAc and H₂O (5 ml each), extraction, separation, extraction of the aqueous layer with EtOAc (4 ml). All organics combined, dried MgSO₄, filtration concentration in vacuo to a brown powder. To this same powder was purified by RP-HPLC (H₂O:AcCN) to afford the title compound as a red powder (36 mg, 48% yield). MS (ES) m/z 459.0; HRMS: calcd for C₂₃H₁₇F₃N₂O₃S+H+, 459.09847. found (ESI, [M+H]⁺ Obs'd), 459.0984.

### Example 3

### 2-methyl-3-{3-[3-(methylsulfonyl)phenoxy]phenyl}-5-(trifluoromethyl)quinoxaline

The title compound was prepared using a procedure analogous to that described in Example 2 but using 3-(3-methyl-8-(trifluoromethyl)quinoxalin-2-yl)phenol in place of 3-[3-methyl-5-(trifluoromethyl)quinoxalin-2-yl]phenol; MS (ES) m/z 459.0; HRMS: calcd for C₂₃H₁₇F₃N₂O₃S+H+, 459.09847. found (ESI, [M+H]+ Obs'd), 459.0988.

### Example 4

### 2-methyl-3-[3′-(methylsulfonyl)biphenyl-4-yl]-5-(trifluoromethyl)quinoxaline

The title compound was prepared using a procedure analogous to that described in Example 1 but using 3-(4-methoxyphenyl)-2-methyl-5-(trifluoromethyl)quinoxaline in place of 2-(4-methoxyphenyl)-3-methyl-5-(trifluoromethyl)quinoxaline; MS (ES) m/z 443.0; HRMS: calcd for C₂₃H₁₇F₃N₂O₂S+H+, 443.10356. found (ESI, [M+H]+ Obs'd), 443.1034.

### Example 5

### 5-chloro-3-methyl-2-{3-[3-(methylsulfonyl)phenoxy]phenyl}quinoxaline

The title compound was prepared using a procedure analogous to that described in Example 2 but using 3-chlorobenzene-1,2-diamine in place of 3-(trifluoromethyl)benzene-1,2-diamine; MS (ES) m/z 390.6; HRMS: calcd for C₂₂H₁₇ClN₂O₃S+H+, 425.07212. found (ESI, [M+H]+ Obs'd), 425.0719.

### Example 6

### 3-[3-fluoro-3′-(methylsulfonyl)biphenyl-4-yl]-2-methyl-5-(trifluoromethyl)quinoxaline

Step 1: 3-(2-fluoro-4-methoxyphenyl)-2-methyl-5-(trifluoromethyl)quinoxaline was prepared using a procedure analogous to that described in Example 1 Step 2 but using 1-(2-fluoro-4-methoxyphenyl)propane-1,2-dione in place of 1-(4-methoxyphenyl)propane-1,2-dione; MS (ESI) m/z 337.1; HRMS: calcd for C₁₇H₁₂F₄N₂O+H+, 337.09585. found (ESI, [M+H]+ Obs'd), 337.0963.

Step 2: 3-fluoro-4-[3-methyl-8-(trifluoromethyl)quinoxalin-2-yl]phenol was prepared using a procedure analogous to that described in Example 1 Step 3 but using 3-(2-fluoro-4-methoxyphenyl)-2-methyl-5-(trifluoromethyl)quinoxaline in place of 2-(4-methoxyphenyl)-3-methyl-5-(trifluoromethyl)quinoxaline; MS (ESI) m/z 323.1; HRMS: calcd for C₁₆H₁₀F₄N₂O+H+, 323.08020. found (ESI, [M+H]+ Obs'd), 323.0802.

Step 3: The title compound was prepared using procedures analogous to those described in Example 1 Step 4 and Step 5 but using 3-fluoro-4-[3-methyl-8-(trifluoromethyl)quinoxalin-2-yl]phenol in place of 4-[3-methyl-5-(trifluoromethyl)quinoxalin-2-yl]phenol; MS (ESI) m/z 461.1; HRMS: calcd for C₂₃H₁₆F₄N₂O₂S+H+, 461.09414. found (ESI, [M+H]+ Obs'd), 461.0943.

### Example 7

### 2-methyl-3-{4-[3-(methylsulfonyl)phenoxy]phenyl}-5-(trifluoromethyl)quinoxaline

The title compound was prepared using a procedure analogous to that described in Example 2 but using 4-(3-methyl-8-(trifluoromethyl)quinoxalin-2-yl)phenol in place of 3-(3-methyl-5-(trifluoromethyl)quinoxalin-2-yl)phenol; MS (ES) m/z 459.0.

### Example 8

### 5-chloro-2-{3-[3-(methylsulfonyl)phenoxy]phenyl}quinoxaline

The title compound was prepared using a procedure analogous to that described in Example 2 but using 2-(4-methoxyphenyl)-2-oxoacetaldehyde in place of 1-(4-methoxyphenyl)propane-1,2-dione; HRMS: calcd for C₂₁H₁₅ClN₂O₃S+H+, 411.05647. found (ESI, [M+H]+ Obs'd), 411.0569.

### Example 9

### 2-{3-[3-(methylsulfonyl)phenoxy]phenyl}-8-(trifluoromethyl)quinoxaline

The title compound was prepared using a procedure analogous to that described in Example 2 but using 2-(4-methoxyphenyl)-2-oxoacetaldehyde in place of 1-(4-methoxyphenyl)propane-1,2-dione; MS (ES) m/z 444.9.

### Example 10

### 5-chloro-2-methyl-3-{3-[3-(methylsulfonyl)phenoxy]phenyl}quinoxaline

The title compound was prepared using a procedure analogous to that described in Example 2 but using 3-(8-chloro-3-methylquinoxalin-2-yl)phenol in place of 3-(3-methyl-5-(trifluoromethyl)-quinoxalin-2-yl)phenol; HRMS: calcd for C₂₂H₁₇ClN₂O₃S+H+, 425.07212. found (ESI, [M+H]+ Obs'd), 425.0719.

### Example 11

### 3-[(3-{3-[8-(trifluoromethyl)quinoxalin-2-yl]phenoxy}phenyl)sulfonyl]propan-1-ol

The title compound was prepared using a procedure analogous to that described in Example 2 but using 4-(8-(trifluoromethyl)quinoxalin-2-yl)phenol and 3-(3-fluorophenylsulfonyl)propan-1-ol in place of 3-(3-methyl-5-(trifluoromethyl)quinoxalin-2-yl)phenol and 1-fluoro-3-(methylsulfonyl)-benzene; MS (ES) m/z 488.9.

### Example 12

### 3-methyl-2-{4-[3-(methylsulfonyl)phenoxy]phenyl}-5-(trifluoromethyl)quinoxaline

The title compound was prepared using a procedure analogous to that described in Example 2 but using 4-(3-methyl-5-(trifluoromethyl)quinoxalin-2-yl)phenol in place of 3-(3-methyl-5-(trifluoro-methyl)quinoxalin-2-yl)phenol; MS (ES) m/z 458.9.

### Example 13

### 8-chloro-2-{3-[3-(methylsulfonyl)phenoxy]phenyl}quinoxaline

The title compound was prepared using a procedure analogous to that described in Example 2 but using 3-(8-chloroquinoxalin-2-yl)phenol in place of 3-(3-methyl-5-(trifluoromethyl)quinoxalin-2-yl)phenol; MS (ES) m/z 410.5.

### Example 14

### 2-{3-[3-(methylsulfonyl)phenoxy]phenyl}-5-(trifluoromethyl)quinoxaline

The title compound was prepared using a procedure analogous to that described in Example 2 but using 3-(5-(trifluoromethyl)quinoxalin-2-yl)phenol in place of 3-(3-methyl-5-(trifluoromethyl)-quinoxalin-2-yl)phenol; MS (ES) m/z 445.0.

### Example 15

### 3-{4-[3-(ethylsulfonyl)phenoxy]phenyl}-2-methyl-5-(trifluoromethyl)quinoxaline

The title compound was prepared using a procedure analogous to that described in Example 2 but using 4-(3-methyl-8-(trifluoromethyl)quinoxalin-2-yl)phenol and 1-(ethylsulfonyl)-3-fluorobenzene in place of 3-(3-methyl-5-(trifluoromethyl)quinoxalin-2-yl)phenol and 1-fluoro-3-(methylsulfonyl)-benzene; MS (ES) m/z 473.0.

### Example 16

### 2-{3-[3-(ethylsulfonyl)phenoxy]phenyl}-8-(trifluoromethyl)quinoxaline

The title compound was prepared using a procedure analogous to that described in Example 2 but using 3-(8-(trifluoromethyl)quinoxalin-2-yl)phenol and 1-(ethylsulfonyl)-3-fluorobenzene in place of 3-(3-methyl-5-(trifluoromethyl)quinoxalin-2-yl)phenol and 1-fluoro-3-(methylsulfonyl)benzene; MS (ES) m/z 459.0.

### Example 17

### 2-{3-[3-(isobutylsulfonyl)phenoxy]phenyl}-8-(trifluoromethyl)quinoxaline

The title compound was prepared using a procedure analogous to that described in Example 2 but using 3-(8-(trifluoromethyl)quinoxalin-2-yl)phenol and 1-fluoro-3-(isobutylsulfonyl)benzene in place of 3-(3-methyl-5-(trifluoromethyl)quinoxalin-2-yl)phenol and 1-fluoro-3-(methylsulfonyl)benzene; MS (ES) m/z 487.0.

### Example 18

### 3-methyl-2-[4′-(methylsulfonyl)biphenyl-4-yl]-5-(trifluoromethyl)quinoxaline

The title compound was prepared using a procedure analogous to that described in Example 1 but using 4-methylsulfonylphenylboronic acid in place of 3-methylsulfonylphenylboronic acid; MS (ES) m/z 443.0; HRMS: calcd for C₂₃H₁₇F₃N₂O₂S+H+, 443.10356. found (ESI, [M+H]+ Obs'd), 443.1039.

### Example 19

### 3-methyl-2-[3′-(methylsulfonyl)biphenyl-3-yl]-5-(trifluoromethyl)quinoxaline

The title compound was prepared using a procedure analogous to that described in Example 1 but using 1-(3-methoxyphenyl)propane-1,2-dione in place of 1-(4-methoxyphenyl)propane-1,2-dione; MS (ES) m/z 443.0; HRMS: calcd for C₂₃H₁₇F₃N₂O₂S+H+, 443.10356. found (ESI, [M+H]+ Obs'd), 443.1037.

### Example 20

### 3-methyl-2-[4′-(methylsulfonyl)biphenyl-3-yl]-5-(trifluoromethyl)quinoxaline

The title compound was prepared using a procedure analogous to that described in Example 1 but using 1-(3-methoxyphenyl)propane-1,2-dione and 4-methylsulfonylphenylboronic acid in place of 1-(4-methoxyphenyl)propane-1,2-dione and 3-methylsulfonylphenylboronic acid; HRMS: calcd for C₂₃H₁₇F₃N₂O₂S+H+, 443.10356. found (ESI, [M+H]+ Obs'd), 443.1038.

### Example 21

### 3-methyl-2-[2′-(methylsulfonyl)biphenyl-4-yl]-5-(trifluoromethyl)quinoxaline

The title compound was prepared using a procedure analogous to that described in Example 1 but using 1-(4-methoxyphenyl)propane-1,2-dione and 2-methylsulfonylphenylboronic acid in place of 1-(3-methoxyphenyl)propane-1,2-dione and 3-methylsulfonylphenylboronic acid; HRMS: calcd for C₂₃H₇F₃N₂O₂S+H+, 443.10356. found (ESI, [M+H]+ Obs'd), 443.1043.

### Example 22

### 2-[3′-(methylsulfonyl)biphenyl-4-yl]-5-(trifluoromethyl)quinoxaline

The title compound was prepared using a procedure analogous to that described in Example 1 but using 2-(4-methoxyphenyl)-2-oxoacetaldehyde in place of 1-(3-methoxyphenyl)propane-1,2-dione; HRMS: calcd for C₂₂H₁₅F₃N₂O₂S+H+, 429.08791. found (ESI, [M+H]+ Obs'd), 429.0883.

### Example 23

### 2-[4′-(methylsulfonyl)biphenyl-4-yl]-8-(trifluoromethyl)quinoxaline

The title compound was prepared using a procedure analogous to that described in Example 1 but using 2-(4-methoxyphenyl)-2-oxoacetaldehyde and 4-methylsulfonylphenylboronic acid in place of 1-(3-methoxyphenyl)propane-1,2-dione and 3-methylsulfonylphenylboronic acid; HRMS: calcd for C₂₂H₁₅F₃N₂O₂S+H+, 429.08791. found (ESI, [M+H]+ Obs'd), 429.0873.

### Example 24

### 2-[2′-(methylsulfonyl)biphenyl-4-yl]-8-(trifluoromethyl)quinoxaline

The title compound was prepared using a procedure analogous to that described in Example 1 but using 2-(4-methoxyphenyl)-2-oxoacetaldehyde and 2-methylsulfonylphenylboronic acid in place of 1-(3-methoxyphenyl)propane-1,2-dione and 3-methylsulfonylphenylboronic acid; HRMS: calcd for C₂₂H₁₅F₃N₂O₂S+H+, 429.08791. found (ESI, [M+H]+ Obs'd), 429.0873.

### Example 25

### 2-[3′-(methylsulfonyl)biphenyl-4-yl]-8-(trifluoromethyl)quinoxaline

The title compound was prepared using a procedure analogous to that described in Example 1 but using 2-(4-methoxyphenyl)-2-oxoacetaldehyde in place of 1-(3-methoxyphenyl)propane-1,2-dione; HRMS: calcd for C₂₂H₁₅F₃N₂O₂S+H+, 429.08791. found (ESI, [M+H]+ Obs'd), 429.0874.

### Example 26

### 2-[3′-(methylsulfonyl)biphenyl-4-yl]-3-(trifluoromethyl)quinoxaline

Step 1: Benzene-1,2-diamine (1.08 g, 10 mmol) and methyl 3,3,3-trifluoro-2-oxopropanoate (1.70 g, 10 mmol) were heated in ethanol (10 mL) to reflux for 1 hour. The reaction was cooled to room temperature and the product was filtered to give 3-(trifluoromethyl)quinoxalin-2-ol as a pale yellow solid (1.50 g); HRMS: calcd for C₉H₅F₃N₂O+H+, 215.04267. found (ESI, [M+H]+ Obs'd), 215.0426.

Step 2: 3-(Trifluoromethyl)quinoxalin-2-ol (1.0 g, 4.67 mmol) and PCl₅ (2 g) was heated to reflux in phosphorus oxychloride (30 mL) for 1 hour. The reaction was concentrated and purified by column chromatography, eluting with a gradient of 0-100% ethyl acetate in hexane to afford 2-chloro-3-(trifluoromethyl)quinoxaline as a white solid (0.6 g).

Step 3: A mixture of 2-chloro-3-(trifluoromethyl)quinoxaline (0.6 g, 2.59 mmol), 4-hydroxylphenyl boronic acid (1.2 g, 8.7 mmol), K₃PO₄ (3.0 g, 14.2 mmol), dicyclohexyl(2′,6′-dimethoxybiphenyl-2-yl)phosphine (0.4 g, 0.98 mmol), Pd(OAc)₂ (0.2 g, 0.90 mmol) in 10 mL of 1-butanol was heated to 80° C. for 30 minutes. The reaction mixture was concentrated and purified by flash chromatography eluted with EtOAc/hexane to give 4-(3-(trifluoromethyl)quinoxalin-2-yl)phenol (0.35) as a pale yellow solid; MS (ESI) m/z 291.1; HRMS: calcd for C₁₅H₉F₃N₂O+H+, 291.07397. found (ESI, [M+H]+ Obs'd), 291.0745.

Step 4: The title compound was prepared using a procedure analogous to that described in Example 1 but using 4-(3-(trifluoromethyl)quinoxalin-2-yl)phenol in place of 4-(3-methyl-5-(trifluoromethyl)quinoxalin-2-yl)phenol; MS (ESI) m/z 429.1; HRMS: calcd for C₂₂H₁₅F₃N₂O₂S+H+, 429.08791. found (ESI, [M+H]+ Obs'd), 429.0877.

### Example 27

### 2-{4-[3-(methylsulfonyl)phenoxy]phenyl}-3-(trifluoromethyl)quinoxaline

The title compound was prepared using a procedure analogous to that described in Example 2 but using 4-(3-(trifluoromethyl)quinoxalin-2-yl)phenol in place of 3-(3-methyl-5-(trifluoromethyl)quinoxalin-2-yl)phenol; MS (ESI) m/z 445.1; HRMS: calcd for C₂₂H₁₅F₃N₂O₃S+H+, 445.08282. found (ESI, [M+H]+ Obs'd), 445.0828.

### Example 28

### 2-[3-fluoro-3′-(methylsulfonyl)biphenyl-4-yl]-3-methyl-5-(trifluoromethyl)quinoxaline

Step 1: 1-(2-Fluoro-4-methoxyphenyl)propan-2-one was oxidized with pyridinium chlorochromate in the presence of pyridine in CH₂Cl₂ to give 1-(2-fluoro-4-methoxyphenyl)propane-1,2-dione (55%).

Step 2: The title compound was prepared using a procedure analogous to that described in Example 1 but using 1-(2-fluoro-4-methoxyphenyl)propane-1,2-dione in place of 1-(4-methoxyphenyl)propane-1,2-dione; MS (ESI) m/z 461.1; HRMS: calcd for C₂₃H₁₆F₄N₂O₂S+H+, 461.09414. found (ESI, [M+H]+ Obs'd), 461.0943.

### Example 29

### 3-{2-fluoro-4-[3-(methylsulfonyl)phenoxy]phenyl}-2-methyl-5-(trifluoromethyl)quinoxaline

The title compound was prepared using a procedure analogous to that described in Example 2 but using 1-(2-fluoro-4-methoxyphenyl)propane-1,2-dione in place of 1-(3-methoxyphenyl)propane-1,2-dione; MS (ESI) m/z 477.1; HRMS: calcd for C₂₃H₁₆F₄N₂O₃S+H+, 477.08905. found (ESI, [M+H]+ Obs'd), 477.0898.

### Example 30

### 2-{2-fluoro-4-[3-(methylsulfonyl)phenoxy]phenyl}-3-methyl-5-(trifluoromethyl)quinoxaline

The title compound was prepared using a procedure analogous to that described in Example 2 but using 1-(2-fluoro-4-methoxyphenyl)propane-1,2-dione in place of 1-(3-methoxyphenyl)propane-1,2-dione; MS (ESI) m/z 477.1; HRMS: calcd for C₂₃H₁₆F₄N₂O₃S+H+, 477.08905. found (ESI, [M+H]+ Obs'd), 477.0895.

### Example 31

### 2-[3′-(ethylsulfonyl)biphenyl-4-yl]-3-methyl-5-(trifluoromethyl)quinoxaline

The title compound was prepared using a procedure analogous to that described in Example 1 but using 3-ethylsulfonylphenylboronic acid in place of 3-methylsulfonylphenylboronic acid; MS (ESI) m/z 457.2; HRMS: calcd for C₂₄H₁₉F₃N₂O₂S+H+, 457.11921. found (ESI, [M+H]+ Obs'd), 457.1199.

### Example 32

### 3-ethyl-2-[3′-(methylsulfonyl)biphenyl-4-yl]-5-(trifluoromethyl)quinoxaline

The title compound was prepared using a procedure analogous to that described in Example 1 but using 1-(4-methoxyphenyl)butane-1,2-dione in place of 1-(4-methoxyphenyl)propane-1,2-dione; MS (ESI) m/z 457.1; HRMS: calcd for C₂₄H₁₉F₃N₂O₂S+H+, 457.11921. found (ESI, [M+H]+ Obs'd), 457.1191.

### Example 33

### 3-[3′-(methylsulfonyl)biphenyl-4-yl]-8-(trifluoromethyl)quinoxalin-2-amine

Step 1: 3-(Trifluoromethyl)benzene-1,2-diamine (1.28 g, 1.46 mmol) and 4-bromobenzaldehyde (0.257 g, 1.46 mmol) were warmed in anhydrous ethanol (30 mL) to 50° C., under a nitrogen atmosphere, for 8 hours. The solvent was removed, in vacuo, to give an amber colored oil (468 mg, 93% Yield). This crude imine was used, as is, in the next reaction.

Step 2: E,Z—N¹-(4-Bromobenzylidene)-3-(trifluoromethyl)benzene-1,2-diamine (150 mg, 0.437 mmol) and potassium cyanide (43 mg, 0.656 mmol) were stirred in anhydrous dimethylformamide at room temperature in a sealed vial for 18 hours. The reaction was partitioned between ethyl acetate and water and the organic phase dried (MgSO₄), filtered and the solvent removed, in vacuo, to give a tan solid which was adsorbed onto silica and purified by column chromatography, eluting with a gradient of 0% to 100% CH₂Cl₂/hexane to afford a light yellow solid (16 mg, 10% Yield). HRMS: calcd for C₁₅H₉BrF₃N₃+H+, 368.0005. found (ESI, [M+H]+, 368.0004.

Step 3: 3-(4-Bromophenyl)-8-(trifluoromethyl)quinoxalin-2-amine (11 mg, 0.030 mmol), 3-(methylsulfonyl)phenylboronic acid (18 mg, 0.090 mmol), potassium phosphate (19 mg, 0.090 mmol), and tetrakis(triphenylphosphine) palladium (0) (10 mg, 0.090 mmol) were heated in anhydrous 1,4-dioxane in a sealed vial to 80° C. for 18 hours. The reaction was allowed to cool to room temperature and adsorbed onto silica and purified by column chormatography, eluting with a gradient of 0-50% ethyl acetate in hexane to afford an off white solid (5 mg, 38% Yield); MS (ESI) m/z 444.1; HRMS: calcd for C₂₂H₁₆F₃N₃O₂S+H+, 444.09881. found (ESI, [M+H]+ Obs'd), 444.0996.

### Example 34

### 2-ethyl-3-[3′-(methylsulfonyl)biphenyl-4-yl]-5-(trifluoromethyl)quinoxaline

The title compound was prepared using a procedure analogous to that described in Example 1 but using 1-(4-methoxyphenyl)butane-1,2-dione in place of 1-(4-methoxyphenyl)propane-1,2-dione; MS (ESI) m/z 457.1; HRMS: calcd for C₂₄H₁₉F₃N₂O₂S+H+, 457.11921. found (ESI, [M+H]+ Obs'd), 457.1200.

### Example 35

### 5,8-dichloro-2-methyl-3-[3′-(methylsulfonyl)biphenyl-4-yl]quinoxaline

Step 1: To a solution of 1-(4-chlorophenyl)propane-1,2-dione (0.2 g, 1.1 mmol) in THF (10 mL) under N₂ was added potassium fluoride (0.19 g, 3.3 mmol), palladium (II) acetate (0.024 g, 0.11 mmol), 2-(dicyclohexylphosphino)biphenyl (0.08 g, 0.22 mmol) and 3-methylsulfonylphenyl boronic acid (0.33 g, 1.65 mmol). The reaction mixture was allowed to stir at room temperature for 18 then diluted with EtOAc, washed with water then dried (Na₂SO₄), filtered and chromatographed on silica (EtOAc/hexane gradient) to give 1-(3′-(methylsulfonyl)biphenyl-4-yl)propane-1,2-dione (0.07 g, 20%) as a yellow oil. MS (ES) m/z 302.9.

Step 2: To a solution of 3,6-dichlorobenzene-1,2-diamine (0.04 g, 0.23 mmol) in EtOH (3 mL) was added 1-(3′-(methylsulfonyl)biphenyl-4-yl)propane-1,2-dione, from step 1, and the reaction mixture allowed to stir for 18 h at room temperature. The reaction was concentrated and chromatographed on silica (EtOAC/hexane gradient) giving 5,8-dichloro-2-methyl-3-[3′-(methylsulfonyl)biphenyl-4-yl]quinoxaline (0.025 g, 25%) as a foamy solid; HRMS: calcd for C₂₂H₁₆O₂N₂O₂S+H+, 443.03823; found (ESI, [M+H]+ Obs'd), 443.0381;

### Example 36

### 2-{2-chloro-5-[3-(methylsulfonyl)phenoxy]phenyl}-3-methyl-5-(trifluoromethyl)quinoxaline

Step 1: A mixture of 1-(3-methoxyphenyl)propan-2-one (4.0 g, 24.4 mmol), Oxone (44.9 g, 73.2 mmol), KCl (5.46 g, 73.2 mmol) in 150 mL of acetonitrile was stirred over night. The solid was filtered off the liquid residue was purified by silica gel chromatropraphy (0 to 70% EtOAc/Hex) to give 1-(2-chloro-5-methoxyphenyl)propan-2-one as an oil (3.4 g, 70%); MS (ESI) m/z 199.0520; HRMS: calcd for C10H11ClO2+H+, 199.05203. found (ESI, [M+H]+), 199.0520.

Step 2: 1-(2-chloro-5-methoxyphenyl)propan-2-one was oxidized with pyridinium chlorochromate in the presence of pyridine in CH₂Cl₂ to give 1-(2-chloro-5-methoxyphenyl)propane-1,2-dione.

Step 3: The title compound was prepared using a procedure analogous to that described in Example 2 but using 1-(2-chloro-5-methoxyphenyl)propane-1,2-dione in place of 1-(3-methoxyphenyl)propane-1,2-dione; MS (ESI) m/z 493.1; HRMS: calcd for C₂₃H₁₆ClF₃N₂O₃S+H+, 493.05950. found (ESI, [M+H]+ Obs'd), 493.0592.

### Example 37

### 3-{2-chloro-5-[3-(methylsulfonyl)phenoxy]phenyl}-2-methyl-5-(trifluoromethyl)quinoxaline

The title compound was prepared using a procedure analogous to that described in Example 2 but using 1-(2-chloro-5-methoxyphenyl)propane-1,2-dione in place of 1-(3-methoxyphenyl)propane-1,2-dione; MS (ESI) m/z 493.1; HRMS: calcd for C₂₃H₁₆ClF₃N₂O₃S+H+, 493.05950. found (ESI, [M+H]+ Obs'd), 493.0592.

### Example 38

### [(4-{4-chloro-3-[3-methyl-8-(trifluoromethyl)quinoxalin-2-yl]phenoxy}phenyl)sulfonyl]-acetonitrile

The title compound was prepared using a procedure analogous to that described in Example 2 but using 1-(2-chloro-5-methoxyphenyl)propane-1,2-dione and 2-(4-fluorophenylsulfonyl)acetonitrile in place of 1-(3-methoxyphenyl)propane-1,2-dione and 1-fluoro-3-(methylsulfonyl)benzene; MS (ESI) m/z 518.1; HRMS: calcd for C₂₄H₁₅ClF₃N₃O₃S+H+, 518.05475. found (ESI, [M+H]+ Obs'd), 518.0548.

### Example 39

### 3-{2-chloro-5-[4-(methylsulfonyl)phenoxy]phenyl}-2-methyl-5-(trifluoromethyl)quinoxaline

The title compound was prepared using a procedure analogous to that described in Example 2 but using 1-(2-chloro-5-methoxyphenyl)propane-1,2-dione in place of 1-(3-methoxyphenyl)propane-1,2-dione; MS (ESI) m/z 493.1.

### Example 40

### 3-methyl-2-{4-[3-(methylsulfonyl)phenoxy]biphenyl-2-yl}-5-(trifluoromethyl)quinoxaline

A mixture of 2-{2-chloro-5-[3-(methylsulfonyl)phenoxy]phenyl}-3-methyl-5-(trifluoromethyl)-quinoxaline (0.017 g, 0.03 mmol), phenyl boronic acid (0.015 g, 0.12 mmol), K₃PO₄ (0.05 g, 0.23 mmol), Pd(OAc)₂ (5 mg), 2-dicyclohexylphosphino-2′,6′-dimethoxybiphenyl (10 mg) in 4 mL of 1-butanol was heated to 80° C. for 30 min. The reaction mixture was purified by flash chromatography eluted with EtOAc/hexane to give the title compound (6 mg) as a white solid; MS (ESI) m/z 535.

### Example 41

### [(4-{4-chloro-3-[3-methyl-5-(trifluoromethyl)quinoxalin-2-yl]phenoxy}phenyl)sulfonyl]-acetonitrile

The title compound was prepared using a procedure analogous to that described in Example 2 but using 1-(2-chloro-5-methoxyphenyl)propane-1,2-dione and 2-(3-fluorophenylsulfonyl)acetonitrile in place of 1-(3-methoxyphenyl)propane-1,2-dione and 1-fluoro-3-(methylsulfonyl)benzene; MS (ESI) m/z 518.1; HRMS: calcd for C₂₄H₁₅ClF₃N₃O₃S+H+, 518.05475. found (ESI, [M+H]+ Obs'd), 518.0544.

### Example 42

### 2-{2-chloro-5-[4-(methylsulfonyl)phenoxy]phenyl}-3-methyl-5-(trifluoromethyl)quinoxaline

The title compound was prepared using a procedure analogous to that described in Example 2 but using 1-(2-chloro-5-methoxyphenyl)propane-1,2-dione and 1-fluoro-4-(methylsulfonyl)benzene in place of 1-(3-methoxyphenyl)propane-1,2-dione and 1-fluoro-3-(methylsulfonyl)benzene; MS (ESI) m/z 493.1; HRMS: calcd for C₂₃H₁₆ClF₃N₂O₃S+H+, 493.05950. found (ESI, [M+H]+ Obs'd), 493.0594.

### Example 44

### 3-{2-chloro-5-[2-(methylsulfonyl)phenoxy]phenyl}-2-methyl-5-(trifluoromethyl)quinoxaline

The title compound was prepared using a procedure analogous to that described in Example 2 but using 1-(2-chloro-5-methoxyphenyl)propane-1,2-dione and 1-fluoro-2-(methylsulfonyl)benzene in place of 1-(3-methoxyphenyl)propane-1,2-dione and 1-fluoro-3-(methylsulfonyl)benzene; MS (ESI) m/z 493.1.

### Example 45

### 3-[(3-{4-chloro-3-[3-methyl-5-(trifluoromethyl)quinoxalin-2-yl]phenoxy}phenyl)sulfonyl]-propan-1-amine

Treatment of 2-{3-[(3-{4-chloro-3-[3-methyl-5-(trifluoromethyl)quinoxalin-2-yl]phenoxy}phenyl)sulfonyl]propyl}-1H-isoindole-1,3(2H)-dione with hydrazine in ethanol gave the title compound; MS (ESI) m/z 535.9.

### Example 46

### 2-{2-chloro-5-[2-(methylsulfonyl)phenoxy]phenyl}-3-methyl-5-(trifluoromethyl)quinoxaline

The title compound was prepared using a procedure analogous to that described in Example 2 but using 1-(2-chloro-5-methoxyphenyl)propane-1,2-dione and 1-fluoro-2-(methylsulfonyl)benzene in place of 1-(3-methoxyphenyl)propane-1,2-dione and 1-fluoro-3-(methylsulfonyl)benzene; MS (ESI) m/z 493.1.

### Example 47

### N-{4′-[3-methyl-8-(trifluoromethyl)quinoxalin-2-yl]biphenyl-3-yl}methanesulfonamide

The title compound was prepared using a procedure analogous to that described in Example 1 but using 3-(methylsulfonamido)phenylboronic acid in place of 3-methylsulfonylphenylboronic acid; MS (ESI) m/z 458.1; HRMS: calcd for C₂₃H₁₈F₃N₃O₂S+H+, 458.11446. found (ESI, [M+H]+ Obs'd), 458.1144.

### Example 48

### N-methyl-4′-[3-methyl-8-(trifluoromethyl)quinoxalin-2-yl]biphenyl-3-sulfonamide

The title compound was prepared using a procedure analogous to that described in Example 1 but using 3-(N-methylsulfamoyl)phenylboronic acid in place of 3-methylsulfonylphenylboronic acid; MS (ESI) m/z 458.2; HRMS: calcd for C₂₃H₁₈F₃N₃O₂S+H+, 458.11446. found (ESI, [M+H]+ Obs'd), 458.1143.

### Example 49

### N-{4′-[3-methyl-5-(trifluoromethyl)quinoxalin-2-yl]biphenyl-3-yl}methanesulfonamide

The title compound was prepared using a procedure analogous to that described in Example 1 but using 3-(methylsulfonamido)phenylboronic acid in place of 3-methylsulfonylphenylboronic acid; MS (ESI) m/z 458.1; HRMS: calcd for C₂₃H₁₈F₃N₃O₂S+H+, 458.11446. found (ESI, [M+H]+ Obs'd), 458.1140.

### Example 50

### 4′-[3-methyl-5-(trifluoromethyl)quinoxalin-2-yl]biphenyl-3-carboxamide

The title compound was prepared using a procedure analogous to that described in Example 1 but using 3-carbamoylphenylboronic acid in place of 3-methylsulfonylphenylboronic acid; MS (ESI) m/z 408.1; HRMS: calcd for C₂₃H₁₆F₃N₃O+H+, 408.13182. found (ESI, [M+H]+ Obs'd), 408.1318.

### Example 51

### N-{4′-[3-methyl-5-(trifluoromethyl)quinoxalin-2-yl]biphenyl-3-yl}acetamide

The title compound was prepared using a procedure analogous to that described in Example 1 but using 3-acetamidophenylboronic acid in place of 3-methylsulfonylphenylboronic acid; MS (ESI) m/z 422.2; HRMS: calcd for C₂₄H₁₈F₃N₃O+H+, 422.14747. found (ESI, [M+H]+ Obs'd), 422.1475.

### Example 52

### N-methyl-4′-[3-methyl-5-(trifluoromethyl)quinoxalin-2-yl]biphenyl-3-carboxamide

The title compound was prepared using a procedure analogous to that described in Example 1 but using 3-(methylcarbamoyl)phenylboronic acid in place of 3-methylsulfonylphenylboronic acid; MS (ESI) m/z 422.2; HRMS: calcd for C₂₄H₁₈F₃N₃O+H+, 422.14747. found (ESI, [M+H]+ Obs'd), 422.1475.

### Example 53

### N-methyl-4′-[3-methyl-5-(trifluoromethyl)quinoxalin-2-yl]biphenyl-3-sulfonamide

The title compound was prepared using a procedure analogous to that described in Example 1 but using 3-(N-methylsulfamoyl)phenylboronic acid in place of 3-methylsulfonylphenylboronic acid; MS (ESI) m/z 458.1; HRMS: calcd for C₂₃H₁₈F₃N₃O₂S+H+, 458.11446. found (ESI, [M+H]+ Obs'd), 458.1142.

### Example 54

### 3-methyl-2-[4′-(pyrrolidin-1-ylcarbonyl)biphenyl-4-yl]-5-(trifluoromethyl)quinoxaline

The title compound was prepared using a procedure analogous to that described in Example 1 but using 3-(pyrrolidine-1-carbonyl)phenylboronic acid in place of 3-methylsulfonylphenylboronic acid; MS (ESI) m/z 462.2; HRMS: calcd for C₂₇H₂₂F₃N₃O+H+, 462.17877. found (ESI, [M+H]+ Obs'd), 462.1789.

### Example 55

### 3-{4′-[3-methyl-5-(trifluoromethyl)quinoxalin-2-yl]biphenyl-4-yl}propanoic acid

The title compound was prepared using a procedure analogous to that described in Example 1 but using 3-(4-boronophenyl)propanoic acid in place of 3-methylsulfonylphenylboronic acid; HRMS: calcd for C₂₅H₁₉F₃N₂O₂+H+, 437.14714. found (ESI, [M+H]+ Obs'd), 437.1469.

### Example 56

### 2-{2-chloro-5-[3-(ethylsulfonyl)phenoxy]phenyl}-3-methyl-5-(trifluoromethyl)quinoxaline

The title compound was prepared using a procedure analogous to that described in Example 36 but using 1-(2-chloro-5-methoxyphenyl)propane-1,2-dione and 1-fluoro-3-(ethylsulfonyl)benzene in place of 1-(3-methoxyphenyl)propane-1,2-dione and 1-fluoro-3-(methylsulfonyl)benzene; HRMS: calcd for C₂₄H₁₈ClF₃N₂O₃S+H+, 507.07515. found (ESI, [M+H]+ Obs'd), 507.0748.

### Example 57

### 2-(2-chloro-5-{3-[(1-methylethyl)sulfonyl]phenoxy}phenyl)-3-methyl-5-(trifluoromethyl)-quinoxaline

The title compound was prepared using a procedure analogous to that described in Example 36 but using 1-(2-chloro-5-methoxyphenyl)propane-1,2-dione and 1-fluoro-3-(isopropylsulfonyl)benzene in place of 1-(3-methoxyphenyl)propane-1,2-dione and 1-fluoro-3-(methylsulfonyl)benzene; MS (ESI) m/z 521.1; HRMS: calcd for C₂₅H₂₀ClF₃N₂O₃S+H+, 521.09080. found (ESI, [M+H]+ Obs'd), 521.0905.

### Example 58

### 3-[(3-{4-chloro-3-[3-methyl-5-(trifluoromethyl)quinoxalin-2-yl]phenoxy}phenyl)sulfonyl]-propan-1-ol

The title compound was prepared using a procedure analogous to that described in Example 36 but using 1-(2-chloro-5-methoxyphenyl)propane-1,2-dione and 3-(3-fluorophenylsulfonyl)propan-1-ol in place of 1-(3-methoxyphenyl)propane-1,2-dione and 1-fluoro-3-(methylsulfonyl)benzene; MS (ESI) m/z 537.1.

### Example 59

### 2-{2-chloro-5-[3-fluoro-5-(methylsulfonyl)phenoxy]phenyl}-3-methyl-5-(trifluoromethyl)-quinoxaline

The title compound was prepared using a procedure analogous to that described in Example 36 but using 1-(2-chloro-5-methoxyphenyl)propane-1,2-dione and 1,3-difluoro-5-(methylsulfonyl)benzene in place of 1-(3-methoxyphenyl)propane-1,2-dione and 1-fluoro-3-(methylsulfonyl)benzene; MS (ESI) m/z 511.1.

### Example 60

### 2-{2-chloro-5-[3-chloro-5-(methylsulfonyl)phenoxy]phenyl}-3-methyl-5-(trifluoromethyl)-quinoxaline

The title compound was prepared using a procedure analogous to that described in Example 36 but using 1-(2-chloro-5-methoxyphenyl)propane-1,2-dione and 1-chloro-3-fluoro-5-(methylsulfonyl)-benzene in place of 1-(3-methoxyphenyl)propane-1,2-dione and 1-fluoro-3-(methylsulfonyl)-benzene; MS (ESI) m/z 527.0.

### Example 61

### 3-{2-chloro-5-[3-(ethylsulfonyl)phenoxy]phenyl}-2-methyl-5-(trifluoromethyl)quinoxaline

The title compound was prepared using a procedure analogous to that described in Example 36 but using 1-(2-chloro-5-methoxyphenyl)propane-1,2-dione and 1-fluoro-3-(ethylsulfonyl)benzene in place of 1-(3-methoxyphenyl)propane-1,2-dione and 1-fluoro-3-(methylsulfonyl)benzene; HRMS: calcd for C₂₄H₁₈ClF₃N₂O₃S+H+, 507.07515. found (ESI, [M+H]+ Obs'd), 507.0747.

### Example 62

### 3-(2-chloro-5-{3-[(1-methylethyl)sulfonyl]phenoxy}phenyl)-2-methyl-5-(trifluoromethyl)quinoxaline

The title compound was prepared using a procedure analogous to that described in Example 36 but using 1-(2-chloro-5-methoxyphenyl)propane-1,2-dione and 1-fluoro-3-(isopropylsulfonyl)benzene in place of 1-(3-methoxyphenyl)propane-1,2-dione and 1-fluoro-3-(methylsulfonyl)benzene; MS (ESI) m/z 521.1; HRMS: calcd for C₂₅H₂₀ClF₃N₂O₃S+Na+, 543.07274. found (ESI, [M+Na]+ Obs'd), 543.0723.

### Example 63

### 3-[(3-{4-chloro-3-[3-methyl-8-(trifluoromethyl)quinoxalin-2-yl]phenoxy}phenyl)sulfonyl]-propan-1-ol

The title compound was prepared using a procedure analogous to that described in Example 36 but using 1-(2-chloro-5-methoxyphenyl)propane-1,2-dione and 3-(3-fluorophenylsulfonyl)propan-1-ol in place of 1-(3-methoxyphenyl)propane-1,2-dione and 1-fluoro-3-(methylsulfonyl)benzene; MS (ESI) m/z 537.1; HRMS: calcd for C₂₅H₂₀ClF₃N₂O₄S+Na+, 559.06766. found (ESI, [M+Na]+ Obs'd), 559.0669.

### Example 64

### 3-{2-chloro-5-[3-fluoro-5-(methylsulfonyl)phenoxy]phenyl}-2-methyl-5-(trifluoromethyl)-quinoxaline

The title compound was prepared using a procedure analogous to that described in Example 36 but using 1-(2-chloro-5-methoxyphenyl)propane-1,2-dione and 1,3-difluoro-5-(methylsulfonyl)benzene in place of 1-(3-methoxyphenyl)propane-1,2-dione and 1-fluoro-3-(methylsulfonyl)benzene; MS (ESI) m/z 511.1; HRMS: calcd for C₂₃H₁₅ClF₄N₂O₃S+Na+, 533.03202. found (ESI, [M+Na]+ Obs'd), 533.0323.

### Example 65

### 3-{2-chloro-5-[3-chloro-5-(methylsulfonyl)phenoxy]phenyl}-2-methyl-5-(trifluoromethyl)-quinoxaline

The title compound was prepared using a procedure analogous to that described in Example 36 but using 1-(2-chloro-5-methoxyphenyl)propane-1,2-dione and 1-chloro-3-fluoro-5-(methylsulfonyl)benzene in place of 1-(3-methoxyphenyl)propane-1,2-dione and 1-fluoro-3-(methylsulfonyl)benzene; MS (ESI) m/z 527.0.

### Example 66

### 2-{2-chloro-5-[3-(methylsulfonyl)phenoxy]phenyl}-3-methylquinoxaline

The title compound was prepared using a procedure analogous to that described in Example 2 but using 1-(2-chloro-5-methoxyphenyl)propane-1,2-dione and benzene-1,2-diamine in place of 1-(3-methoxyphenyl)propane-1,2-dione and 3-(trifluoromethyl)benzene-1,2-diamine; MS (ESI) m/z 425.1; HRMS: calcd for C₂₂H₁₇ClN₂O₃S+H+, 425.07212. found (ESI, [M+H]+ Obs'd), 425.0727.

### Example 67

### 5-chloro-3-methyl-2-[3′-(methylsulfonyl)biphenyl-4-yl]quinoxaline

The title compound was prepared using a procedure analogous to that described in Example 1 but using 3-chlorobenzene-1,2-diamine in place of 3-(trifluoromethyl)benzene-1,2-diamine; MS (ESI) m/z 409.1; HRMS: calcd for C₂₂H₁₇ClN₂O₂S+H+, 409.07720. found (ESI, [M+H]+ Obs'd), 409.0774.

### Example 68

### 5-chloro-2-methyl-3-[3′-(methylsulfonyl)biphenyl-4-yl]quinoxaline

The title compound was prepared using a procedure analogous to that described in Example 1 but using 3-chlorobenzene-1,2-diamine in place of 3-(trifluoromethyl)benzene-1,2-diamine; MS (ESI) m/z 409.1; HRMS: calcd for C₂₂H₁₇ClN₂O₂S+H+, 409.07720. found (ESI, [M+H]+ Obs'd), 409.0775.

### Example 69

### 5-methoxy-2-methyl-3-[3′-(methylsulfonyl)biphenyl-4-yl]quinoxaline

The title compound was prepared using a procedure analogous to that described in Example 1 but using 3-methoxybenzene-1,2-diamine in place of 3-(trifluoromethyl)benzene-1,2-diamine; MS (ESI) m/z 405.1; HRMS: calcd for C₂₃H₂₀N₂O₃S+H+, 405.12674. found (ESI, [M+H]+ Obs'd), 405.1259.

### Example 70

### 5-methoxy-3-methyl-2-[3′-(methylsulfonyl)biphenyl-4-yl]quinoxaline

The title compound was prepared using a procedure analogous to that described in Example 1 but using 3-methoxybenzene-1,2-diamine in place of 3-(trifluoromethyl)benzene-1,2-diamine; MS (ESI) m/z 405.2; HRMS: calcd for C₂₃H₂₀N₂O₃S+H+, 405.12674. found (ESI, [M+H]+ Obs'd), 405.1262.

### Example 71

### 3-methyl-2-[3′-(methylsulfonyl)biphenyl-4-yl]quinoxaline-5-carbonitrile

The title compound was prepared using a procedure analogous to that described in Example 1 but using 2,3-diaminobenzonitrile in place of 3-(trifluoromethyl)benzene-1,2-diamine; HRMS: calcd for C₂₃H₁₇N₃O₂S+H+, 400.11142. found (ESI, [M+H]+ Obs'd), 400.1115.

### Example 72

### 2-methyl-3-[3′-(methylsulfonyl)biphenyl-4-yl]quinoxaline-5-carbonitrile

The title compound was prepared using a procedure analogous to that described in Example 1 but using 2,3-diaminobenzonitrile in place of 3-(trifluoromethyl)benzene-1,2-diamine; HRMS: calcd for C₂₃H₁₇N₃O₂S+H+, 400.11142. found (ESI, [M+H]+ Obs'd), 400.1114.

### Example 73

### 2-methyl-3-{4-[5-(methylsulfonyl)pyridin-3-yl]phenyl}-5-(trifluoromethyl)quinoxaline

The title compound was prepared using a procedure analogous to that described in Example 1 but using 5-(methylsulfonyl)pyridin-3-ylboronic acid in place of 3-(methylsulfonyl)phenylboronic acid; MS (ESI) m/z 444.1; HRMS: calcd for C₂₂H₁₆F₃N₃O₂S+H+, 444.09881. found (ESI, [M+H]+ Obs'd), 444.0997.

### Example 74

### 3-methyl-2-{4-[5-(methylsulfonyl)pyridin-3-yl]phenyl}-5-(trifluoromethyl)quinoxaline

The title compound was prepared using a procedure analogous to that described in Example 1 but using 5-(methylsulfonyl)pyridin-3-ylboronic acid in place of 3-(methylsulfonyl)phenylboronic acid; MS (ESI) m/z 444.1; HRMS: calcd for C₂₂H₁₆F₃N₃O₂S+H+, 444.09881. found (ESI, [M+H]+ Obs'd), 444.0991.

### Example 75

### 2-(2-chloro-5-{3-[(3-fluoropropyl)sulfonyl]phenoxy}phenyl)-3-methyl-5-(trifluoromethyl)-quinoxaline

Step 1: In a flash at 0° C. under a nitrogen atmosphere was placed 3-(3-(4-chloro-3-(3-methyl-5-(trifluoromethyl)quinoxalin-2-yl)phenoxy)phenylsulfonyl)propan-1-ol (660 mg, 1.229 mmol) in CH₂Cl₂ (20 ml) followed by in order methanesulfonyl chloride (155 mg, 1.352 mmol), triethylamine (148 mg, 1.478 mmol). The resulting yellow solution was stirred at 0° C. for 30 min. Complete conversion was confirmed by LCMS upon which the reaction was quenched with H₂O (10 ml). Extraction, separation, drying with MgSO₄, and concentration in vacuo of the organic phase to a yellow syrup. Purification of this same syrup by chromatography SiO₂ (elution with Hexane: EtOAc) afforded 3-[(3-{4-chloro-3-[3-methyl-5-(trifluoromethyl)quinoxalin-2-yl]phenoxy}phenyl)sulfonyl]propyl methanesulfonate as a yellow powder (592 mg, 78% yield); MS (ESI) m/z 615.1. HRMS: calcd for C₂₆H₂₂ClF₃N₂O₆S₂+H+, 615.06326. found (ESI, [M+H]+ Obs'd), 615.0639.

Step 2: In an open reaction vial at room temperature was placed 3-[(3-{4-chloro-3-[3-methyl-5-(trifluoromethyl)quinoxalin-2-yl]phenoxy}phenyl)sulfonyl]propyl methanesulfonate (40 mg, 0.065 mmol) and potassium fluoride (4 mg, 0.071 mmol) in DMF (1 ml). The reaction vial was capped and heated at 70° C. for 3 h. Complete conversion was confirmed by LCMS upon which the reaction was quenched with H₂O (5 ml). Partition with EtOAc (5 ml) extraction, separation, extraction of the aqueous layer with EtOAc (3 ml). All organics were combined, dried with MgSO₄, and concentration in vacuo of the organic phase to a yellow powder. This same yellow powder was purified by reversed phase chromatography (CH₃CN:H₂O) affording the title compound as a white powder (9 mg, 26% yield). MS (ESI) m/z 539.1.

### Example 76

### 2-(2-chloro-5-{3-[(3-chloropropyl)sulfonyl]phenoxy}phenyl)-3-methyl-5-(trifluoromethyl)-quinoxaline

The title compound was prepared using a procedure analogous to that described in Example 75 but using sodium chloride in place of potassium fluoride; MS (ESI) m/z 555.1. HRMS: calcd for C₂₅H₁₉Cl₂F₃N₂O₃S+H+, 555.05183. found (ESI, [M+H]+ Obs'd), 555.0526.

### Example 77

### 4-[(3-{4-chloro-3-[3-methyl-5-(trifluoromethyl)quinoxalin-2-yl]phenoxy}phenyl)sulfonyl]-butanenitrile

The title compound was prepared using a procedure analogous to that described in Example 75 but using potassium cyanide in place of potassium fluoride; MS (ESI) m/z 546.1. HRMS: calcd for C₂₆H₁₉ClF₃N₃O₃S+H+, 546.08605. found (ESI, [M+H]+ Obs'd), 546.0866.

### Example 78

### 5-chloro-2-{[3′-(methylsulfonyl)biphenyl-3-yl]oxy}quinoxaline

Step 1: 3-chloro-2-nitroaniline (5.0 g, 29 mmol), zinc dust (18.9 g, 290 mmol) and ammonium chloride (31.0 g, 579 mmol) were stirred in 100 mL of a 1:1 solution of ethanol and water, at room temperature for 18 hours. The reaction was filtered and the filtrate concentrated to give a brown solid, which was transferred to a separatory funnel and partitioned between methylene chloride and water. The organic extracts were separated, combined, dried (MgSO₄), filtered and the solvent removed, in vacuo, to give 3-chlorobenzene-1,2-diamine as a brown solid (4.00 g, 97% Yield).

Step 2: 3-chlorobenzene-1,2-diamine (3.60 g, 25.3 mmol) and ethyl glyoxylate solution (50% in toluene; 6.0 mL, 30.3 mmol) were heated in ethanol (87 mL) to 75° C. for 18 hours. The reaction was placed in a refrigerator to cool and the product filtered to give a rust colored solid (3.42 g). This material was purified by supercritical fluid chromatography to give 5-chloroquinoxalin-2(1H)-one; MS (ESI) m/z 180.0.

Step 3: 5-chloroquinoxalin-2(1H)-one (0.60 g, 3.32 mmol) was heated to 90° C. in phosphorus oxychloride (10 mL, 109 mmol) for 3 hours. The reaction was poured onto ice and extracted with ethyl acetate. The organic extracts were combined, dried (MgSO₄), filtered and the solvent removed to give a dark brown solid. This material was adsorbed onto silica and purified by column chromatography, eluting with a gradient of 0-30% ethyl acetate in hexane to afford 2,5-dichloroquinoxaline as a white solid (210 mg, 32% Yield).

Step 4: 2,5-dichloroquinoxaline (90 mg, 0.452 mmol), 3-bromophenol (86 mg, 0.497 mmol) and potassium carbonate (82 mg, 0.542 mmol) were heated to 90° C. in anhydrous acetonitrile (3 mL) for 18 hours. The reaction was allowed to cool to room temperature and transferred to a separatory funnel with ethyl acetate and washed with water, 1N aqueous sodium hydroxide solution, brine, dried (MgSO₄), filtered, and the solvent removed in vacuo, to give an off white solid. This material was adsorbed onto silica and purified by column chromatography, eluting with a gradient of 0-25% ethyl acetate in hexane to afford 2-(3-bromophenoxy)-5-chloroquinoxaline as a white solid (130 mg, 86% Yield); MS (ESI) m/z 333.95087; HRMS: calcd for C₁₄H₈BrClN₂O+H+, 334.9581. found (ESI, [M+H]+ Obs'd), 334.9583.

Step 5: 2-(3-bromophenoxy)-5-chloroquinoxaline (50 mg, 0.149 mmol), 3-methylsulfonylphenyl-boronic acid (45 mg, 0.223 mmol), tetrakis(triphenylphosphine) palladium (0) (17 mg, 0.0149 mmol), and 2M aqueous sodium carbonate solution (149 μL, 0.298 mmol) were heated to 80° C. in a 1.5 mL of a 2:1 solution of toluene:ethanol for 18 hours. The reaction was filtered through celite and the filtrate adsorbed onto silica and purified by column chromatography eluting with a gradient of 0-75% ethyl acetate in hexane to give impure product which was purified by supercritical fluid chromatography to give the title compound as a white solid (26 mg, 43% Yield); MS (ESI) m/z 411.1; HRMS: calcd for C₂₁H₁₅ClN₂O₃S+H+, 411.05647. found (ESI, [M+H]+ Obs'd), 411.0567.

### Example 79

### 5-chloro-2-{[3′-(methylsulfonyl)biphenyl-4-yl]oxy}quinoxaline

Step 1: 2-(4-bromophenoxy)-5-chloroquinoxaline was prepared using 4-bromophenol and utilizing essentially the same conditions as Example 78 Step 4; MS (ESI) m/z [M+H]+334.9; HRMS: calcd for C₁₄H₈BrClN₂O+H+, 334.9581. found (ESI, [M+H]+ Obs'd), 334.9582.

Step 2: The title compound was prepared utilizing essentially the same conditions as Example 78 Step 5; MS (ESI) m/z 411.1; HRMS: calcd for C₂₁H₁₅ClN₂O₃S+H+, 411.05647. found (ESI, [M+H]+ Obs'd), 411.0564.

### Example 80

### 5-chloro-3-methyl-2-{[3′-(methylsulfonyl)biphenyl-3-yl]oxy}quinoxaline

Step 1: 5-chloro-3-methylquinoxalin-2(1H)-one was prepared using ethyl pyruvate and utilizing essentially the same conditions as Example 78 Step 2; MS (ESI) m/z 195.0; HRMS: calcd for C₉H₇ClN₂O+H+, 195.0320. found (ESI, [M+H]+ Obs'd), 195.0321.

Step 2: 2,5-dichloro-3-methylquinoxaline was prepared using 5-chloro-3-methylquinoxalin-2(1H)-one and utilizing essentially the same conditions as Example 78 Step 3; MS (ESI) m/z [M+H]+213.0.

Step 3: 2-(3-bromophenoxy)-5-chloro-3-methylquinoxaline was prepared using 2,5-dichloro-3-methylquinoxaline and utilizing essentially the same conditions as Example 78 Step 4; MS (ESI) m/z [M+H]+ 349.0; HRMS: calcd for C₁₅H₁₀BrClN₂O+H+, 348.9738. found (ESI, [M+H]+ Obs'd), 348.9737.

Step 4: The title compound was prepared utilizing essentially the same conditions as Example 78 Step 5; MS (ESI) m/z 425.1; HRMS: calcd for C₂₂H₁₇ClN₂O₃S+H+, 425.07212. found (ESI, [M+H]+ Obs'd), 425.0718.

### Example 81

### 5-chloro-2-{[3′-(ethylsulfonyl)biphenyl-3-yl]oxy}-3-methylquinoxaline

The title compound was prepared using 3-ethylsulfonylphenylboronic acid and utilizing essentially the same conditions as Example 80; MS (ESI) m/z 439.1; HRMS: calcd for C₂₃H₁₉ClN₂O₃S+H+, 439.08777. found (ESI, [M+H]+ Obs'd), 439.0881.

### Example 82

### 5-chloro-3-methyl-2-{[3′-(methylsulfonyl)biphenyl-4-yl]oxy}quinoxaline

Step 1: 2-(4-bromophenoxy)-5-chloro-3-methylquinoxaline was prepared utilizing essentially the same conditions as Example 78 Step 4; MS (ESI) m/z [M+H]+ 349.0; HRMS: calcd for C₁₅H₁₀BrClN₂O+H+, 348.9738. found (ESI, [M+H]+ Obs'd), 348.9735.

Step 2: The title compound was prepared utilizing essentially the same conditions as Example 78 Step 5; MS (ESI) m/z 425.1; HRMS: calcd for C₂₂H₁₇ClN₂O₃S+H+, 425.07212. found (ESI, [M+H]+ Obs'd), 425.0720.

### Example 83

### 5-chloro-2-{[3′-(ethylsulfonyl)biphenyl-4-yl]oxy}-3-methylquinoxaline

The title compound was prepared utilizing essentially the same conditions as Example 80 but using 3-ethylsulfonylphenylboronic acid in place of 3-methylsulfonylphenylboronic acid; MS (ESI) m/z 439.1; HRMS: calcd for C₂₃H₁₉ClN₂O₃S+H+, 439.08777. found (ESI, [M+H]+ Obs'd), 439.0876.

### Example 84

### 3′-[(5-Chloroquinoxalin-2-yl)oxy]-N-methylbiphenyl-3-sulfonamide

Step 1: 3-Chloro-2-nitroaniline (5.0 g, 29 mmol), zinc (18.9 g, 290 mmol) and ammonium chloride (31.0 g, 579 mmol) were stirred in 100 mL of a 1:1 solution of ethanol and water, at room temperature for 18 hours. The reaction was filtered and the filtrate concentrated to give a brown solid which was transferred to a separatory funnel and partitioned between methylene chloride and water. The organic extracts were separated, combined, dried (MgSO₄), filtered and the solvent removed, in vacuo, to give 3-chlorobenzene-1,2-diamine as a brown solid (4.00 g, 97% yield).

Step 2: 3-Chlorobenzene-1,2-diamine (3.60 g, 25.3 mmol) and ethyl glyoxylate solution (50% in toluene; 6.0 mL, 30.3 mmol) were heated in ethanol (87 mL) to 75° C. for 18 hours. The reaction was placed in a refrigerator to cool and the product filtered to give 5-chloroquinoxalin-2(1H)-one as a rust colored solid (3.42 g). This material was purified by supercritical fluid chromatography to give 5-chloroquinoxalin-2(1H)-one and 8-chloroquinoxalin-2(1H)-one.

Step 3: 5-Chloroquinoxalin-2(1H)-one (0.60 g, 3.32 mmol) was heated to 90° C. in phosphorus oxychloride (10 mL, 109 mmol) for 3 hours. The reaction was poured onto ice and extracted with ethyl acetate. The organic extracts were combined, dried (MgSO₄), filtered and the solvent removed to give a dark brown solid. This material was adsorbed onto silica and purified by column chromatography, eluting with a gradient of 0-30% ethyl acetate in hexane to afford 2,5-dichloroquinoxaline as a white solid (210 mg, 32% yield).

Step 4: 2,5-Dichloroquinoxaline (90 mg, 0.452 mmol), 3-bromophenol (86 mg, 0.497 mmol) and potassium carbonate (82 mg, 0.542 mmol) were heated to 90° C. in anhydrous acetonitrile (3 mL) for 18 hours. The reaction was allowed to cool to room temperature and transferred to a separatory funnel with ethyl acetate and washed with water, 1N aqueous sodium hydroxide solution, brine, dried (MgSO₄), filtered, and the solvent removed in vacuo, to give an off white solid. This material was adsorbed onto silica and purified by column chromatography, eluting with a gradient of 0-25% ethyl acetate in hexane to afford 2-(3-bromophenoxy)-5-chloroquinoxaline as a white solid (130 mg, 86% yield); MS (ESI) m/z 334.0; HRMS: calcd for C₁₄H₈BrClN₂O+H+, 334.9581. found (ESI, [M+H]+ Obs'd), 334.9583.

Step 5: 2-(3-Bromophenoxy)-5-chloroquinoxaline (25 mg, 0.0745 mmol), methyl-3-boronobenzenesulfamide (24 mg, 0.0112 mmol), tetrakis(triphenylphosphine) palladium (0) (9 mg, 0.0745 mmol), and 2M aqueous sodium carbonate solution (74 μL, 0.149 mmol) were heated to 80° C. in a 1.5 mL of a 2:1 solution of toluene:ethanol for 18 hours. The reaction was filtered through celite and the filtrate adsorbed onto silica and purified by column chromatography eluting with a gradient of 0-50% ethyl acetate in hexane to afford the title compound as a white solid (11 mg, 34% yield). MS (ESI) m/z 425.0; HRMS: calcd for C₂₁H₁₆ClN₃O₃S+H+, 426.0674. found (ESI, [M+H]+ Obs'd), 426.0673.

### Example 85

### N-{3′-[(5-Chloroquinoxalin-2-yl)oxy]biphenyl-3-yl}methanesulfonamide

The title compound was prepared followed the same procedure as described in Example 84 Step 5 using 3-(methylsulfonamido)phenylboronic acid instead of methyl-3-boronobenzenesulfamide as a white solid (13 mg, 41% yield). MS (ESI) m/z 425.0; HRMS: calcd for C₂₁H₁₆ClN₃O₃S+H+, 426.0674. found (ESI, [M+H]+ Obs'd), 426.0672.

### Example 86

### 4′-[(5-Chloroquinoxalin-2-yl)oxy]-N-methylbiphenyl-3-sulfonamide

Step 1: 2-(4-Bromophenoxy)-5-chloroquinoxaline was prepared using 4-bromophenol and utilizing the same conditions as Example 78 Step 4 as a white solid (27 mg, 40% yield). MS (ESI) m/z 334.0; HRMS: calcd for C₁₄H₈BrClN₂O+H+, 334.9581. found (ESI, [M+H]+ Obs'd), 334.9582.

Step 2: The title compound was prepared followed the same procedure as described in Example 84 using 2-(4-bromophenoxy)-5-chloroquinoxaline instead of 2-(3-bromophenoxy)-5-chloro-quinoxaline as a white solid (21 mg, 55% Yield). MS (ESI) m/z 425.0; HRMS: calcd for C₂₁H₁₆ClN₃O₃S+H+, 426.0674. found (ESI, [M+H]+ Obs'd), 426.0675.

### Example 87

### N-{4′-[(5-chloroquinoxalin-2-yl)oxy]biphenyl-3-yl}methanesulfonamide

The title compound was prepared followed the same procedure as described in Example 85 using 2-(4-bromophenoxy)-5-chloroquinoxaline instead of 2-(3-bromophenoxy)-5-chloroquinoxaline as a white solid (20 mg, 53% Yield). MS (ESI) m/z 425.0; HRMS: calcd for C₂₁H₁₆ClN₃O₃S+H+, 426.0674. found (ESI, [M+H]+ Obs'd), 426.0675.

### Example 88

### 3′-[(5-chloro-3-methylquinoxalin-2-yl)oxy]-N-methylbiphenyl-3-sulfonamide

Step 1: 5-Chloro-3-methylquinoxalin-2(1H)-one was prepared using ethyl pyruvate and utilizing the same conditions as in the preparation of Example 84 Step 2. MS (ESI) m/z 195.0; HRMS: calcd for C₉H₇ClN₂O+H+, 195.0320. found (ESI, [M+H]+ Obs'd), 195.0321.

Step 2: 2,5-Dichloro-3-methylquinoxaline was prepared using 5-chloro-3-methylquinoxalin-2(1H)-one and utilizing the same conditions as in the preparation of Example 84 Step 3. MS (ESI) m/z [M+H]+213.0.

Step 3: 2-(3-Bromophenoxy)-5-chloro-3-methylquinoxaline was prepared using 2,5-dichloro-3-methylquinoxaline and utilizing the same conditions as in the preparation of Example 84 Step 4. MS (ESI) m/z [M+H]+ 349.0; HRMS: calcd for C₁₅H₁₀BrClN₂O+H+, 348.9738. found (ESI, [M+H]+ Obs'd), 348.9737.

Step 4: The title compound was prepared using the same procedure as described in Example 84 Step 5 using 2-(3-bromophenoxy)-5-chloro-3-methylquinoxaline instead of 2-(3-bromophenoxy)-5-chloroquinoxaline as a pink colored solid (33 mg, 52% Yield). MS (ESI) m/z 439.1; HRMS: calcd for C₂₂H₁₈ClN₃O₃S+H+, 440.0830. found (ESI, [M+H]+ Obs'd), 440.0827.

### Example 89

### 3′-[(5-chloro-3-methylquinoxalin-2-yl)oxy]biphenyl-3-sulfonamide

The title compound was prepared followed the same procedure as described in Example 88 using 3-sulfamoylphenylboronic acid instead of methyl-3-boronobenzenesulfamide as a pink colored solid (31 mg, 51% Yield). MS (ESI) m/z 425.1; HRMS: calcd for C₂₁H₁₆ClN₃O₃S+H+, 426.0674; found (ESI, [M+H]+ Obs'd), 426.0675.

### Example 90

### N-{3′-[(5-chloro-3-methylquinoxalin-2-yl)oxy]biphenyl-3-yl}methanesulfonamide

The title compound was prepared followed the same procedure as described in Example 88 using 3-methylsulfonylaminophenylboronic acid instead of methyl-3-boronobenzenesulfamide as a pink colored solid (38 mg, 60% Yield). MS (ESI) m/z 439.0; HRMS: calcd for C₂₂H₁₈ClN₃O₃S+H+, 440.0830. found (ESI, [M+H]+ Obs'd), 440.0830.

### Example 91

### 4′-[(5-chloro-3-methylquinoxalin-2-yl)oxy]-N-methylbiphenyl-3-sulfonamide

The title compound was prepared followed the same procedure as described in Example 84 Step 5 using 5-chloro-3-methylquinoxalin-2(1H)-one instead of 5-chloroquinoxalin-2(1H)-one as a pink colored solid (38 mg, 60% Yield). MS (ESI) m/z 439.0; HRMS: calcd for C₂₂H₁₈ClN₃O₃S+H+, 440.0830. found (ESI, [M+H]+ Obs'd), 440.0831.

### Example 92

### 4′-[(5-chloro-3-methylquinoxalin-2-yl)oxy]biphenyl-3-sulfonamide

The title compound was prepared followed the same procedure as described in Example 91 using 3-sulfamoylphenylboronic acid instead of methyl-3-boronobenzenesulfamide as a white solid (24 mg, 39% Yield). MS (ESI) m/z 425.1; HRMS: calcd for C₂₁H₁₆ClN₃O₃S+H+, 426.0674. found (ESI, [M+H]+ Obs'd), 426.0675.

### Example 93

### N-{4′-[(5-chloro-3-methylquinoxalin-2-yl)oxy]biphenyl-3-yl}methanesulfonamide

The title compound was prepared followed the same procedure as described in Example 91 using 3-methylsulfonylaminophenylboronic acid instead of methyl-3-boronobenzenesulfamide as a white solid (38 mg, 60% Yield). MS (ESI) m/z 439.1;

HRMS: calcd for C₂₂H₁₈ClN₃O₃S+H+, 440.0830. found (ESI, [M+H]+ Obs'd), 440.0827.

The structures of the title compounds of Examples 1-42 and 44-83 are set forth below.

| E.g. # | Structure of Title compound |
|---|---|
| 1 | 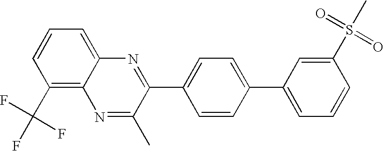 |
| 2 | 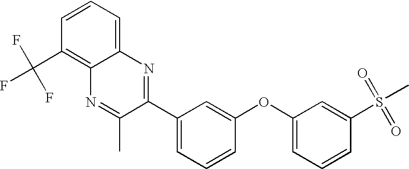 |
| 3 | 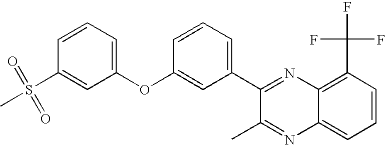 |
| 4 | 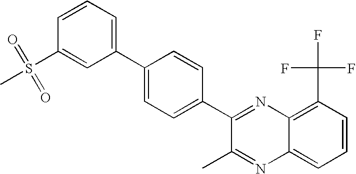 |
| 5 | 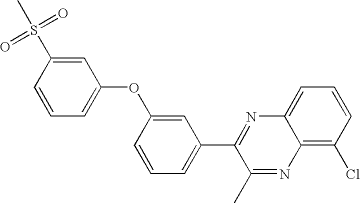 |
| 6 | 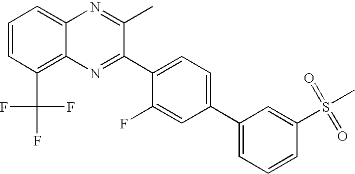 |
| 7 | 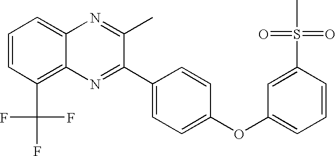 |
| 8 | 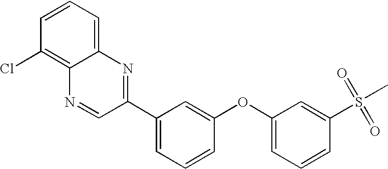 |
| 9 | 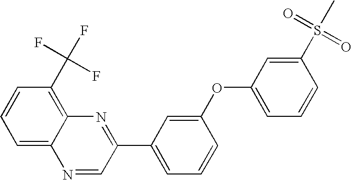 |
| 10 | 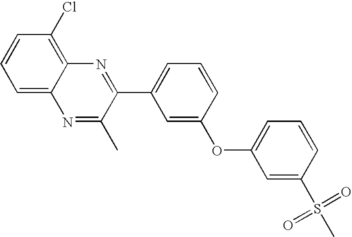 |
| 11 | 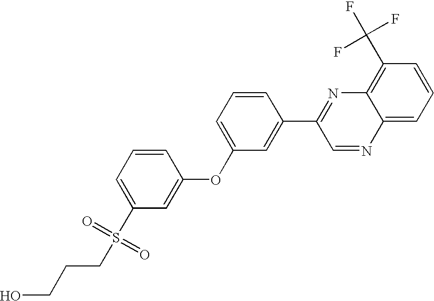 |
| 12 | 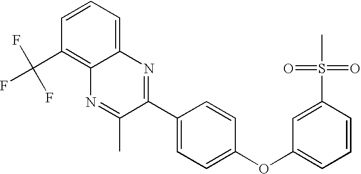 |
| 13 | 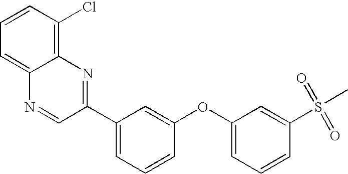 |
| 14 | 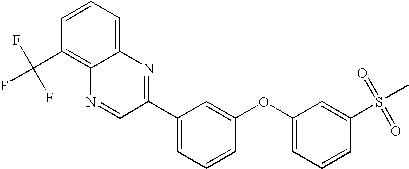 |
| 15 | 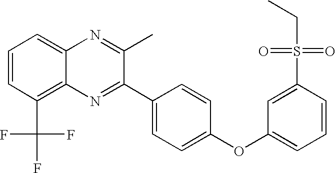 |
| 16 | 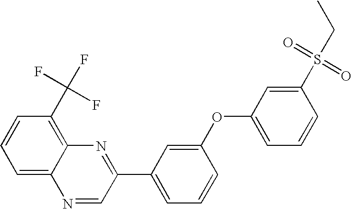 |
| 17 | 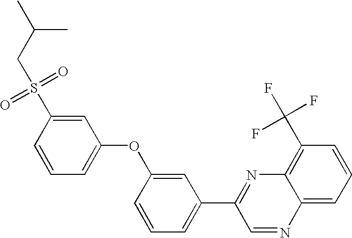 |
| 18 | 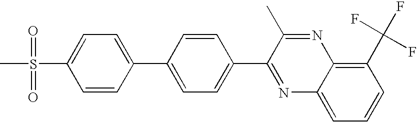 |
| 19 | 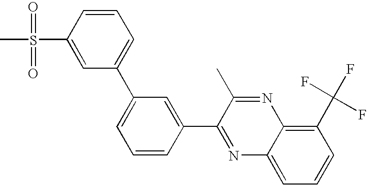 |
| 20 | 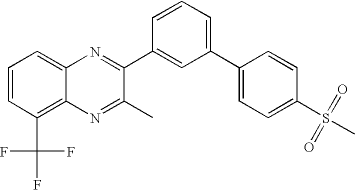 |
| 21 | 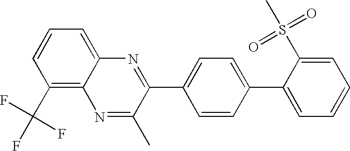 |
| 22 | 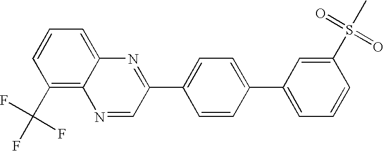 |
| 23 | 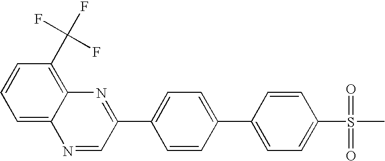 |
| 24 | 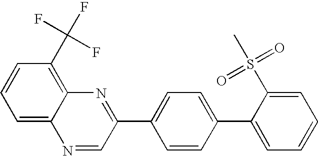 |
| 25 | 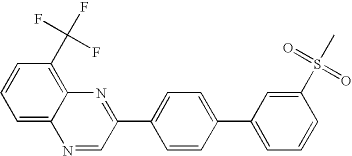 |
| 26 | 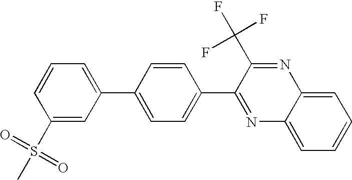 |
| 27 | 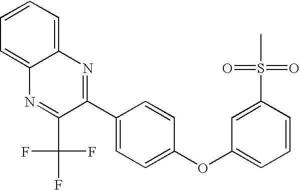 |
| 28 | 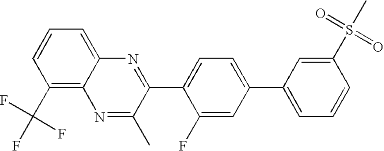 |
| 29 | 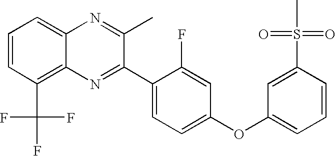 |
| 30 | 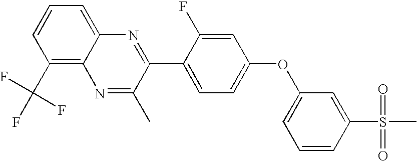 |
| 31 | 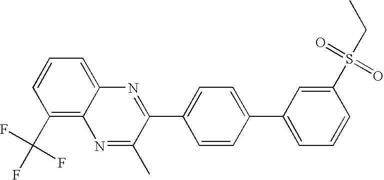 |
| 32 | 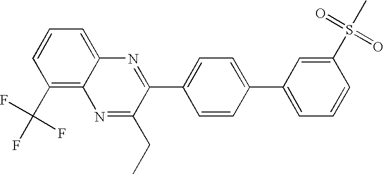 |
| 33 | 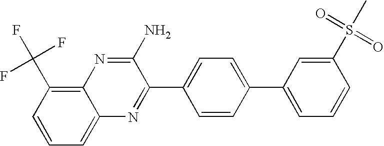 |
| 34 | 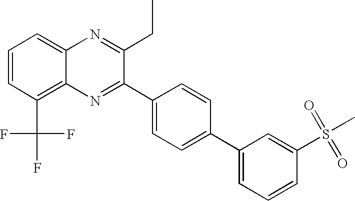 |
| 35 | 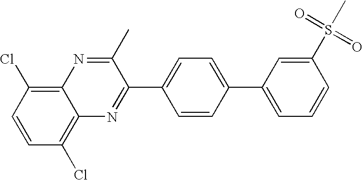 |
| 36 | 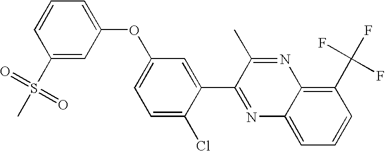 |
| 37 | 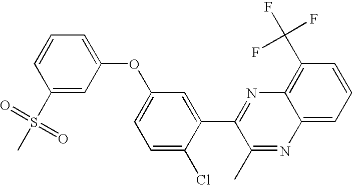 |
| 38 | 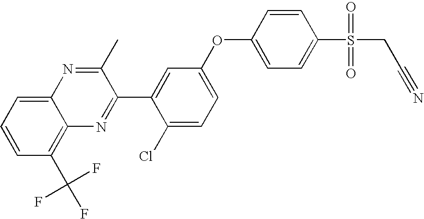 |
| 39 | 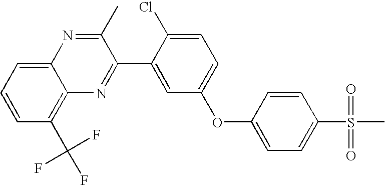 |
| 40 | 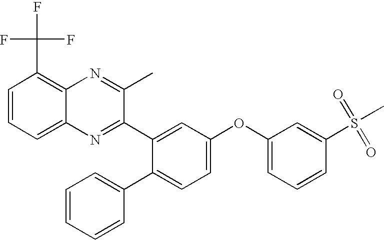 |
| 41 | 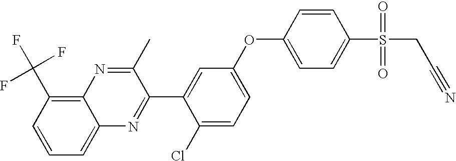 |
| 42 | 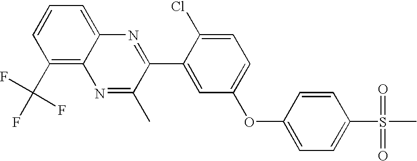 |
| 43 | This field intentionally left blank. |
| 44 | 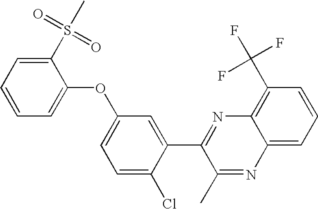 |
| 45 | 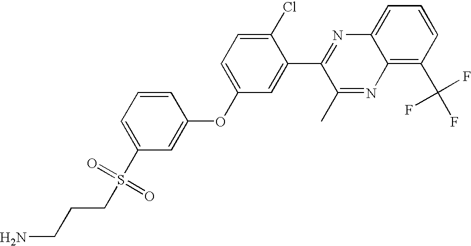 |
| 46 | 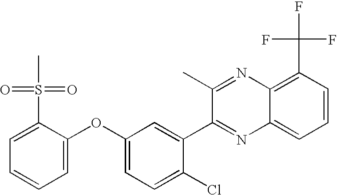 |
| 47 | 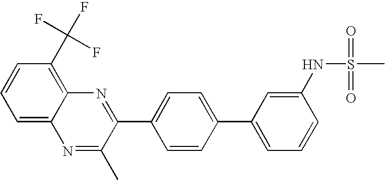 |
| 48 | 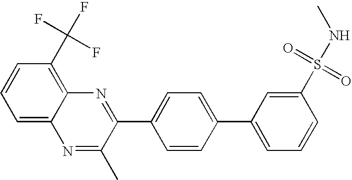 |
| 49 | 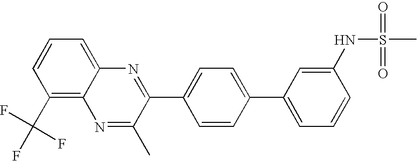 |
| 50 | 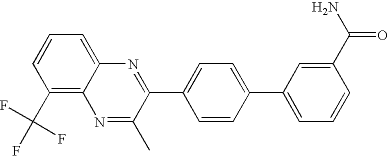 |
| 51 | 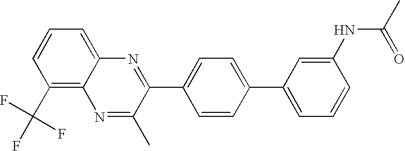 |
| 52 | 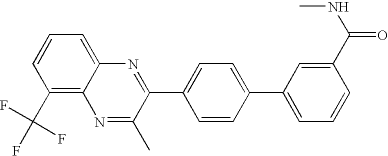 |
| 53 | 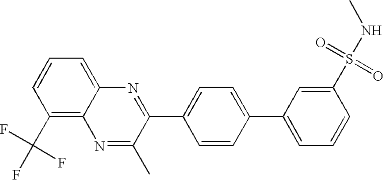 |
| 54 | 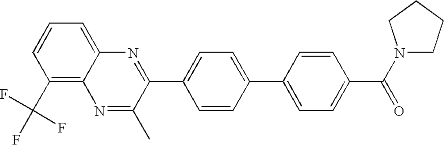 |
| 55 | 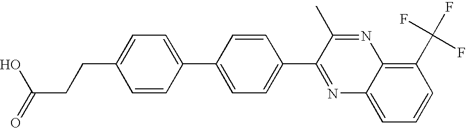 |
| 56 | 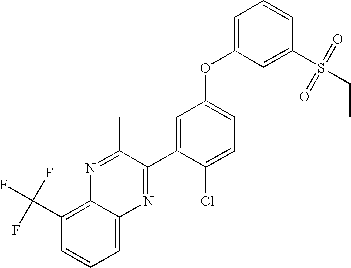 |
| 57 | 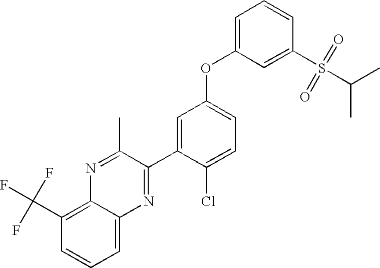 |
| 58 | 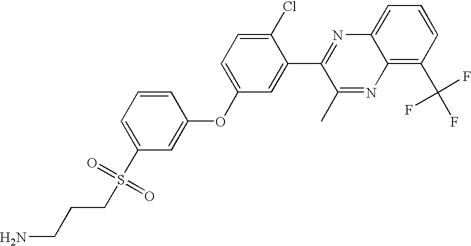 |
| 59 | 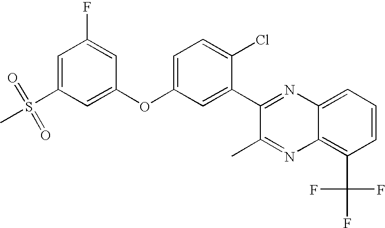 |
| 60 | 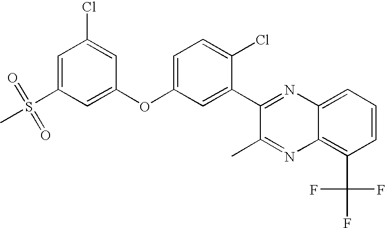 |
| 61 | 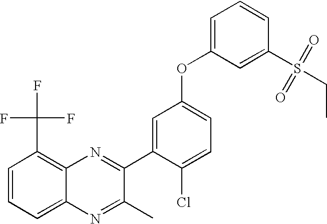 |
| 62 | 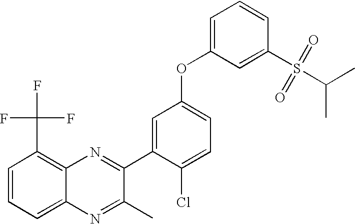 |
| 63 | 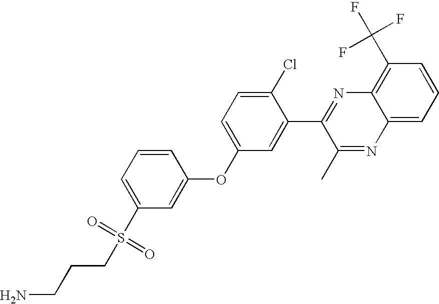 |
| 64 | 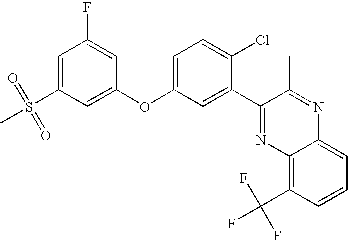 |
| 65 | 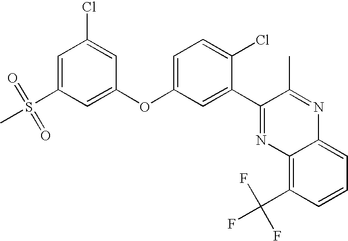 |
| 66 | 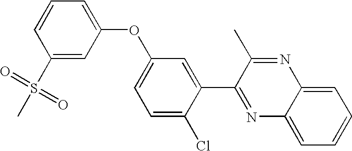 |
| 67 | 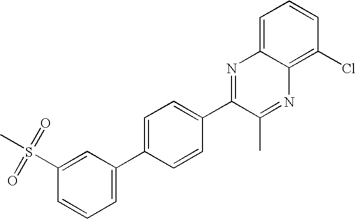 |
| 68 | 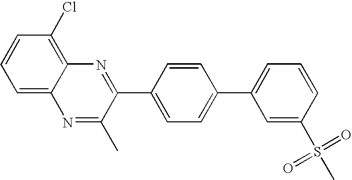 |
| 69 | 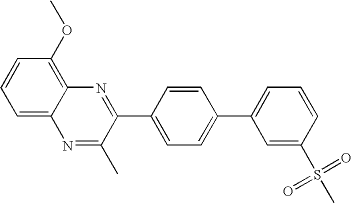 |
| 70 | 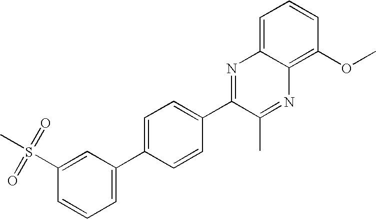 |
| 71 | 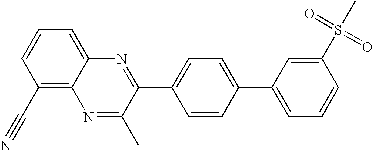 |
| 72 | 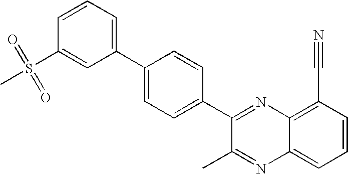 |
| 73 | 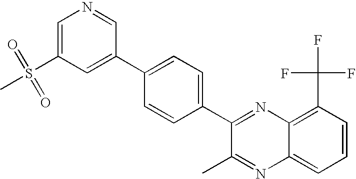 |
| 74 | 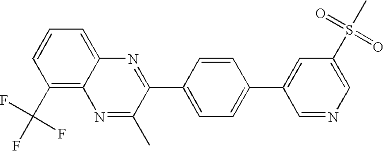 |
| 75 | 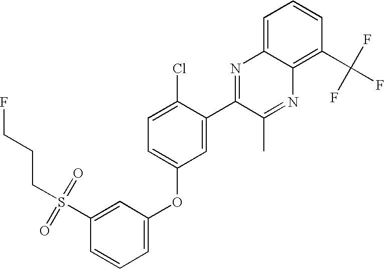 |
| 76 | 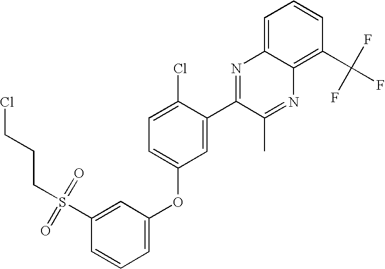 |
| 77 | 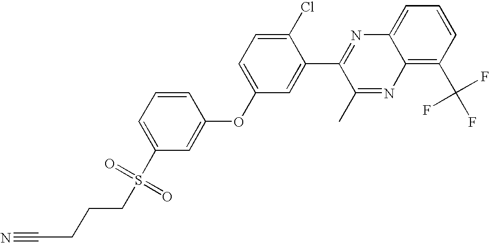 |
| 78 | 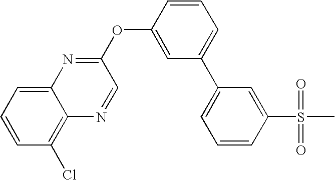 |
| 79 | 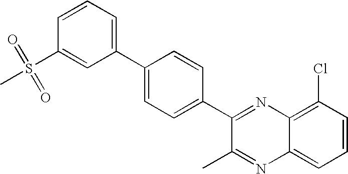 |
| 80 | 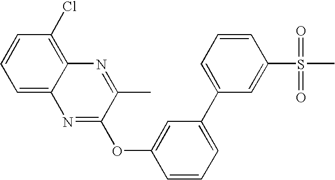 |
| 81 | 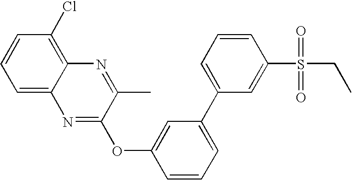 |
| 82 | 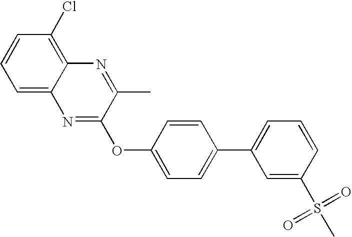 |
| 83 | 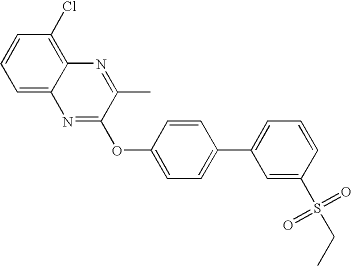 |
| 84 | 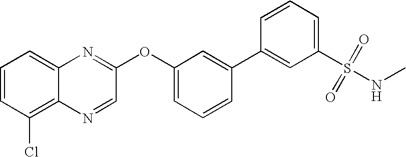 |
| 85 | 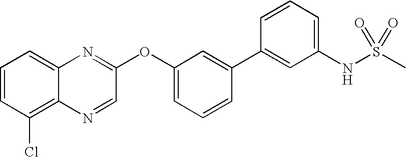 |
| 86 | 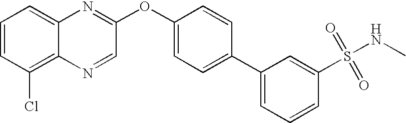 |
| 87 | 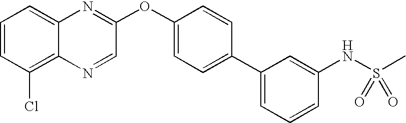 |
| 88 | 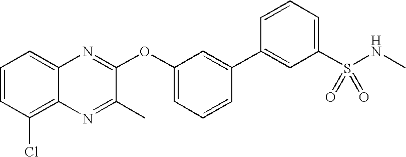 |
| 89 | 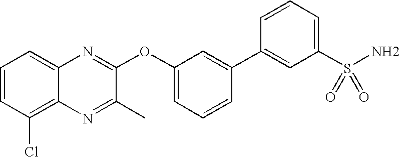 |
| 90 | 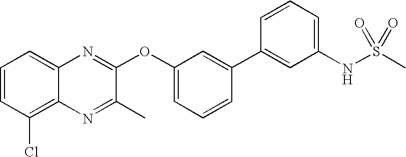 |
| 91 | 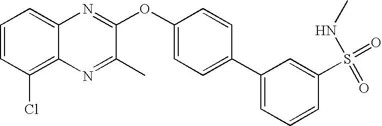 |
| 92 | 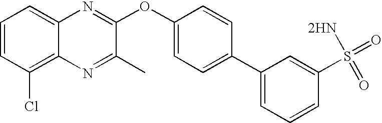 |
| 93 | 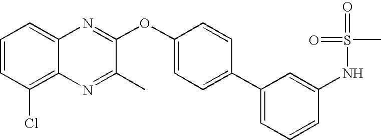 |

### Biological Testing

Representative compounds of this invention were evaluated in conventional pharmacological test procedures which measured their affinity to bind to LXR and to upregulate the gene ABCA1, which causes cholesterol efflux from atherogenic cells, such as macrophages.

LXR activation can be critical for maintaining cholesterol homeostasis, but its coincident regulation of fatty acid metabolism may lead to increased serum and hepatic triglyceride levels. Selective LXR modulators that activate cholesterol efflux with minimal impact on SREBP-1c expression and triglyceride synthesis in liver would be expected to reduce atherosclerotic risk with an improved therapeutic index and minimize the potential for deleterious effects on metabolic balance.

The test procedures performed, and results obtained, are briefly described in the following sections:

I. Ligand-Binding Test Procedure for Human LXRβ

II. Ligand-Binding Test Procedure for Human LXRα

III. Quantitative Analysis of ABCA1 Gene Regulation in THP-1 Cells

IV. Results

### I. Ligand-Binding Test Procedure for Human LXRβ.

Ligand-binding to the human LXRβ was demonstrated for representative compounds of this invention by the following procedure.

### Materials and Methods:

Buffer: 100 mM KCl, 100 mM TRIS (pH 7.4 at +4° C.), 8.6% glycerol, 0.1 mM PMSF*, 2 mM MTG*, 0.2% CHAPS (* not used in wash buffer)

### Tracer: ³H T0901317

Receptor source: E. coli extracted from cells expressing biotinylated hLXRβ. Extract was made in a similar buffer as above, but with 50 mM TRIS.

### Day 1

Washed streptavidin and coated flash plates with wash buffer.
Diluted receptor extract to give Bₘₐₓ˜4000 cpm and add to the wells.
Wrapped the plates in aluminum foil and stored them at +4° C. overnight.

### Day 2

Made a dilution series in DMSO of the test ligands.
Made a 5 nM solution of the radioactive tracer in buffer.
Mixed 250 μl diluted tracer with 5 μl of the test ligand from each concentration of the dilution series.
Washed the receptor-coated flash plates.
Added 200 μl per well of the ligand/radiolabel mixture to the receptor-coated flash plates.
Wrapped the plates in aluminum foil and incubate at +4° C. over night.

### Day 3

Aspirated wells, and washed the flashed plates. Sealed the plate.

Measured the remaining radioactivity in the plate.

### II. Ligand-Binding Test Procedure for Human LXRα.

Ligand-binding to the human LXRα was demonstrated for representative compounds of this invention by the following procedure.

### Materials and Methods:

Buffer: 100 mM KCl, 100 mM TRIS (pH 7.4 at +4° C.), 8.6% glycerol, 0.1 mM PMSF*, 2 mM MTG*, 0.2% CHAPS (* not used in wash buffer)

### Tracer: ³H T0901317

Receptor source: E. coli extract from cells expressing biotinylated hLXRα. Extract was made in a similar buffer as above, but with 50 mM TRIS.

### Day 1

Washed streptavidin and coated flash plates with wash buffer.
Diluted receptor extract to give Bmax˜4000 cpm and add to the wells.
Wrapped the plates in aluminum foil and stored them at +4° C. over night.

### Day 2

Made a dilution series in DMSO of the test ligands.
Made a 5 nM solution of the radioactive tracer in buffer.
Mixed 250 μl diluted tracer with 5 μl of the test ligand from each concentration of the dilution series.
Washed the receptor-coated flash plates.
Added 200 μl per well of the ligand/radiolabel mixture to the receptor-coated flash plates.
Wrapped the plates in aluminum foil and incubate at +4° C. over night.

### Day 3

Aspirated wells, and wash the flashed plates. Sealed the plate.
Measured the remaining radioactivity in the plate.

### III. Quantitative Analysis of ABCA1 Gene Regulation In THP-1 Cells.

The compounds of formula (I) effect on the regulation of the ABCA1 gene was evaluated using the following procedure.

### Materials and Methods

Cell culture: The THP-1 monocytic cell line (ATCC # TIB-202) was obtained from American Type Culture Collection (Manassas, Va.) and cultured in RPMI 1640 medium (Gibco, Carlsbad, Calif.) containing 10% FBS, 2 mM L-glutamine, and 55 uM beta-Mercaptoethanol (BME). Cells were plated in 96-well format at a density of 7.5×10⁴ in complete medium containing 50-100 ng/ml phorbal 12,13-dibutyrate (Sigma, St. Louis, Mo.) for three days to induce differentiation into adherent macrophages. Differentiated THP-1 cells were treated with test compounds or ligands dissolved in DMSO (Sigma, D-8779) in culture medium lacking phorbal ester. Final concentrations of DMSO did not exceed 0.3% of the media volume. Dose response effects were measured in duplicate, in the range of 0.001 to 30 micromolar concentrations and treated cells were incubated for an additional 18 hrs prior to RNA isolation. Unstimulated cells treated with vehicle were included as negative controls on each plate. An LXR agonist reference, N-(2,2,2-Trifluoro-ethyl)-N-[4-(2,2,2-trifluoro-1-hydroxy-1-trifluoromethyl-ethyl)-phenyl]-benzenesulfonamide (Schultz, Joshua R., Genes & Development (2000), 14(22), 2831-2838), was dosed at 1.0 uM and served as a positive control. In antagonist mode, the compound under study is analyzed in the presence of 150 nM GW3965, trifluoromethyl-benzyl)-(2,2-diphenyl-ethyl)-amino]-propoxy]-phenyl)-acetic acid (Collins, J. L., J. Med. Chem. (2000), 45:1963-1966.). Results of antagonist analysis are expressed as % antagonism and IC50 (in μM).

RNA isolation and quantitation: Total cellular RNA was isolated from treated cells cultured in 96-well plates using PrepStation 6100 (Applied Biosystems, Foster City, Calif.), according to the manufacturer's recommendations. RNA was resuspended in ribonuclease-free water and stored at −70° C. prior to analysis. RNA concentrations were quantitated with RiboGreen test procedure, #R-11490 (Molecular Probes, Eugene, Oreg.).

Gene expression analysis: Gene-specific mRNA quantitation was performed by real-time PCR with the Perkin Elmer Corp. chemistry on an ABI Prism 7700 Sequence detection system (Applied Biosystems, Foster City, Calif.) according to the manufacturer's instructions. Samples (50-100 ng) of total RNA were assayed in duplicate or triplicate in 50 μl reactions using one-step RT-PCR and the standard curve method to estimate specific mRNA concentrations. Sequences of gene-specific primer and probe sets were designed with Primer Express Software (Applied Biosystems, Foster City, Calif.). The human ABCA1 primer and probe sequences are: forward, CAACATGAATGCCATTTTCCAA, reverse, ATAATCCCCTGAACCCAAGGA, and probe, 6FAM-TAAAGCCATGCCCTCTGCAGGAACA-TAMRA. RT and PCR reactions were performed according to PE Applied Biosystem's protocol for Taqman Gold RT-PCR or Qiagen's protocol for Quantitect probe RT-PCR. Relative levels of ABCA1 mRNA are normalized using GAPDH mRNA or 18S rRNA probe/primer sets purchased commercially (Applied Biosystems, Foster City, Calif.).

### Statistics:

Mean, standard deviation and statistical significance of duplicate evaluations of RNA samples were assessed using ANOVA, one-way analysis of variance using SAS analysis.

### Reagents:

### GAPDH Probe and Primers—Taqman GAPDH Control Reagents 402869 or 4310884E

### 18S Ribosomal RNA—Taqman 18S Control Reagents 4308329

### 10 Pack Taqman PCR Core Reagent Kit 402930

Qiagen Quantitect probe RT-PCR 204443.

### IV. Results:

TABLE I

| | hLXRβ binding | hLXRα binding |
|---|---|---|
| Example | IC50 (μM) | IC50 (μM) |
| 1 | 0.049 | 0.752 |
| 2 | 0.1272 | 1.017 |
| 3 | 0.251 | 1.293 |
| 4 | 0.373 | >1.0 |
| 5 | 0.39 | >1.0 |
| 6 | 0.398 | >1.0 |
| 7 | 1.549 | >1.0 |
| 8 | 1.646 | >1.0 |
| 9 | 1.958 | >1.0 |
| 10 | 2.211 | >1.0 |
| 11 | 2.484 | >1.0 |
| 12 | 6.395 | >1.0 |
| 13 | >1.0 | |
| 14 | >1.0 | |
| 15 | >1.0 | |
| 16 | >1.0 | |
| 17 | >1.0 | |
| 18 | >1.0 | |
| 19 | >1.0 | |
| 20 | >1.0 | |
| 21 | >1.0 | |
| 22 | >1.0 | |
| 23 | >1.0 | |
| 24 | >1.0 | |
| 25 | >1.0 | |
| 26 | >1.0 | |
| 27 | >1.0 | |
| 28 | >1.0 | |
| 29 | >1.0 | |
| 30 | >1.0 | |
| 31 | 0.13202065 | 0.265 |
| 32 | 0.01802535 | 0.26545375 |
| 33 | 0.7226969 | |
| 34 | 0.3533397 | |
| 35 | 0.0994506 | |
| 36 | 0.0195 | 0.292 |
| 37 | 0.037 | 0.557 |
| 38 | >1.0 | |
| 39 | >1.0 | |
| 40 | >1.0 | |
| 41 | >1.0 | |
| 42 | 0.745 | 1.894 |
| 44 | >1.0 | |
| 45 | 0.063 | |
| 46 | 1.456 | 1.617 |
| 47 | >1 | |
| 48 | 1.09 | |
| 49 | >1 | |
| 50 | >1 | |
| 51 | >1 | |
| 52 | >1 | |
| 53 | 0.081 | >1 |
| 54 | >1 | |
| 55 | >1 | |
| 56 | 0.012 | 0.435 |
| 57 | 0.0789 | 0.947 |
| 58 | 0.0635 | 0.81 |
| 59 | 0.032 | 0.249 |
| 60 | 0.159 | >1 |
| 61 | 0.029 | 0.366 |
| 62 | 0.038 | 0.876 |
| 63 | 0.048 | 0.979 |
| 64 | 0.089 | 0.638 |
| 65 | 0.453 | >1 |
| 66 | 0.361 | >1 |
| 67 | 0.03 | 0.554 |
| 68 | 0.235 | >1 |
| 69 | >1 | |
| 70 | 0.395 | >1 |
| 71 | 0.265 | >1 |
| 72 | 3.023 | >1 |
| 73 | 0.123 | >1 |
| 74 | >1 | >1 |
| 75 | 0.287 | 2.187 |
| 76 | 0.204 | 0.401 |
| 77 | 0.128 | 0.405 |
| 78 | 0.263 | >1 |
| 79 | >1 | >1 |
| 80 | 0.108 | 0.818 |
| 81 | 0.24 | >1 |
| 82 | 1.027 | >1 |
| 83 | >1 | >1 |
| 84 | 0.64 | |
| 85 | 0.257 | |
| 86 | >1 | |
| 87 | >1 | |
| 88 | >1 | |
| 89 | >1 | |
| 90 | >1 | |
| 91 | 0.933 | |
| 92 | 2.467 | |
| 93 | 0.869 | 3.528 |

TABLE II

Gene regulation by LXR (THP1)

| | EC50 ABCA1 | Agonism ABCA1 |
|---|---|---|
| Example | (μM) | (%) |
| 1 | 1.094 | 79% |
| 31 | 0.89 | 65% |
| 53 | 1.4 | 55% |
| 77 | 4.42 | 45% |

Based on the results obtained in the standard pharmacological test procedures, the compounds of this invention can be useful in treating or inhibiting LXR mediated diseases. In particular, the compounds of this invention can be useful in the treatment or prevention of atherosclerosis and atherosclerotic lesions, lowering LDL cholesterol levels, increasing HDL cholesterol levels, increasing reverse cholesterol transport, inhibiting cholesterol absorption, treatment or inhibition of cardiovascular diseases (e.g., acute coronary syndrome, restenosis, coronary artery disease), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging (e.g., skin aging is derived from chronological aging, photoaging, steroid-induced skin thinning, or a combination thereof), or connective tissue disease (e.g., osteoarthritis or tendonitis).

A number of embodiments of the invention have been described. Nevertheless, it will be understood that various modifications may be made without departing from the spirit and scope of the invention. Accordingly, other embodiments are in the claims.

What is claimed is:

1. A compound having formula (I): 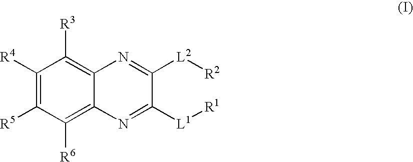
wherein:
each of L¹ and L² is, independently, a bond, —O— or —NH—;
R¹ is:
(i) hydrogen; or
(ii) C₁-C₆ alkyl or C₁-C₆ haloalkyl, each of which is optionally substituted with from 1-3 R^{a}; or
(iii) C₂-C₆ alkenyl or C₂-C₆ alkynyl, each of which is optionally substituted with from 1-3 R^{b}; or
(iv) C₃-C₇ cycloalkyl optionally substituted with from 1-3 R^{c}; or
(v) NR⁷R⁸, wherein R⁷ and R⁸ at each occurrence is, independently, hydrogen, C₁-C₆ alkyl, or C₃-C₇ cycloalkyl;
R² is C₆-C₁₀ aryl or heteroaryl including 5-10 atoms, each of which is:
(i) substituted with 1 R⁹, and
(ii) optionally further substituted with from 1-4 R^{e}; wherein:
R⁹ is WA, wherein:
W at each occurrence is, independently, a bond; —O—; —NR¹⁰— wherein R¹⁰ is hydrogen, C₁-C₆ alkyl, or C₃-C₇ cycloalkyl; C₁₋₆ alkylene, C₂₋₆ alkenylene, or C₂₋₆ alkynylene; or —(C₁₋₆ alkylene)W¹—;
W¹ at each occurrence is, independently, —O—, —NH— or —N(C₁₋₆ alkyl)-;
A at each occurrence is, independently, C₆-C₁₀ aryl or heteroaryl including 5-10 atoms, each of which is:
(i) substituted with 1 R¹¹, and
(ii) optionally further substituted with from 1-4 R^{g};
R¹¹ at each occurrence is, independently:
(i) —W²—S(O)ₙR¹² or —W²—S(O)ₙNR¹³R¹⁴; or
(ii) —W²—C(O)OR¹⁵; or
(iii) —W²—C(O)NR¹³R¹⁴; or
(iv) —W²—CN; or
(v) C₁-C₈ alkyl or C₁-C₈ haloalkyl, each of which is:
(a) substituted with 1 R^{h}, and
(b) optionally further substituted with from 1-3 R^{a}; or
(vi) —NR¹⁶R¹⁷;
wherein:
W² at each occurrence is, independently, a bond; C₁₋₆ alkylene; C₂₋₆ alkenylene;
C₂₋₆ alkynylene; C₃₋₆ cycloalkylene; —O(C₁₋₆ alkylene)-; —NH(C₁₋₆ alkylene)-; or —N(C₁-C₆ alkyl)(C₁₋₆ alkylene)-;
n at each occurrence is, independently, 1 or 2;
R¹² at each occurrence is, independently:
(i) C₁-C₆ alkyl or C₁-C₆ haloalkyl, each of which is optionally substituted with from 1-3 R^{a}; or
(ii) C₂-C₆ alkenyl or C₂-C₆ alkynyl, each of which is optionally substituted with from 1-3 R^{b}; or
(iii) C₃-C₈ cycloalkyl, C₃-C₈ cycloalkenyl, C₇-C₁₁ aralkyl, or heteroaralkyl including 6-11 atoms, each of which is optionally substituted with from 1-3 R^{c}; or
(iv) C₆-C₁₀ aryl or heteroaryl including 5-10 atoms, each of which is optionally substituted with from 1-3 R^{d};
R¹³ and R¹⁴ are each, independently, hydrogen; R¹² or heterocyclyl including 3-8 atoms or a heterocycloalkenyl including 3-10 atoms, each of which is optionally substituted with from 1-3 R^{c}; or
R¹³ and R¹⁴ together with the nitrogen atom to which they are attached form a heterocyclyl including 3-8 atoms or a heterocycloalkenyl including 3-8 atoms, each of which is optionally substituted with from 1-3 R^{c};
R¹⁵ at each occurrence is, independently, hydrogen or R¹²;
one of R¹⁶ and R¹⁷ is hydrogen or C₁-C₃ alkyl; and the other of R¹⁶ and R¹⁷ is:
(i) —S(O)ₙR¹²; or
(ii) —C(O)R¹²; or
(iii) —C(O)OR¹³; or
(iv) —C(O)NR¹³R¹⁴; or
(v) C₁-C₈ alkyl or C₁-C₈ haloalkyl, each of which is:
(a) substituted with 1 R^{h}, and
(b) optionally further substituted with from 1-3 R^{a};
each of R³ and R⁶ is, independently:
(i) hydrogen; or
(ii) halo; or
(iii) C₁-C₆ alkyl or C₁-C₆ haloalkyl, each of which is optionally substituted with from 1-3 R^{a}; or
(iv) nitro; hydroxy; C₁-C₆ alkoxy; C₁-C₆ haloalkoxy; C₁-C₆ thioalkoxy; C₁-C₆ thiohaloalkoxy; cyano; or S(O)_{z}(C₁-C₃ alkyl), wherein z is 1 or 2;
each of R⁴ and R⁵ is, independently:
(i) hydrogen; or
(ii) halo; or
(iii) C₁-C₆ alkyl or C₁-C₆ haloalkyl, each of which is optionally substituted with from 1-3 R^{a};
R^{a} at each occurrence is, independently:
(i) NR^{m}Rⁿ; hydroxy; C₁-C₆ alkoxy or C₁-C₆ haloalkoxy; or
(ii) C₃-C₇ cycloalkyl optionally substituted with from 1-3 substituents independently selected from NR^{m}Rⁿ; hydroxy; C₁-C₆ alkyl, C₁-C₆ alkoxy and C₁-C₆ haloalkoxy;
R^{b} at each occurrence is, independently:
(i) halo; NR^{m}Rⁿ; hydroxy; C₁-C₆ alkoxy or C₁-C₆ haloalkoxy; or
(ii) C₃-C₇ cycloalkyl optionally substituted with from 1-3 substituents independently selected from NR^{m}Rⁿ; hydroxy; C₁-C₆ alkyl, C₁-C₆ alkoxy and C₁-C₆ haloalkoxy;
R^{c} at each occurrence is, independently:
(i) halo; NR^{m}Rⁿ; hydroxy; C₁-C₆ alkoxy or C₁-C₆ haloalkoxy; or
(ii) C₁-C₆ alkyl or C₁-C₆ haloalkyl; or
(iii) C₂-C₆ alkenyl or C₂-C₆ alkynyl;
R^{d} at each occurrence is, independently:
(i) halo; NR^{m}Rⁿ; hydroxy; C₁-C₆ alkoxy or C₁-C₆ haloalkoxy; or cyano; or
(ii) C₁-C₆ alkyl or C₁-C₆ haloalkyl, each of which is optionally substituted with from 1-3 R^{a}; or
(iii) C₂-C₆ alkenyl or C₂-C₆ alkynyl, each of which is optionally substituted with from 1-3 R^{b};
R^{e} at each occurrence is, independently, C₁-C₆ alkyl; C₁-C₆ haloalkyl; halo; hydroxy; NR^{m}Rⁿ; C₁-C₆ alkoxy; C₁-C₆ haloalkoxy; cyano; or phenyl, which is optionally substituted with from 1-4 R^{d};
R^{g} at each occurrence is, independently, C₁-C₆ alkyl; C₁-C₆ haloalkyl; halo; hydroxy; NR^{m}Rⁿ; C₁-C₆ alkoxy; C₁-C₆ haloalkoxy; or cyano;
R^{h} at each occurrence is, independently, hydroxyl, C₁-C₆ alkoxy, or C₁-C₆ haloalkoxy; C₃-C₈ cycloalkoxy or C₃-C₈ cycloalkenyloxy, each of which is optionally substituted with from 1-3 R^{c}; or C₆-C₁₀ aryloxy or heteroaryloxy including 5-10 atoms, each of which is optionally substituted with from 1-3 R^{d};
each of R^{m} and Rⁿ at each occurrence is, independently, hydrogen, C₁-C₆ alkyl, or C₁-C₆ haloalkyl;
or an N-oxide and/or a pharmaceutically acceptable salt thereof.

2. The compound of claim 1, wherein each of L¹ and L² is a bond.

3. The compound of claim 1, wherein one of L¹ and L² is a bond, and the other of L¹ and L² is —O—.

4. The compound of claim 1, wherein R¹ is C₁-C₆ alkyl or C₁-C₆ haloalkyl.

5. The compound of claim 4, wherein R¹ is C₁-C₃ alkyl.

6. The compound of claim 5, wherein R¹ is CH₃.

7. The compound of claim 1, wherein R² is C₆-C₁₀ aryl, which is (a) substituted with 1 R⁹; and (b) optionally further substituted with from 1-4 R^{e}.

8. The compound of claim 1, wherein R² is phenyl, which is (a) substituted with 1 R⁹; and (b) optionally further substituted with from 1-4 R^{e}.

9. The compound of claim 8, wherein R² has formula (A-2): 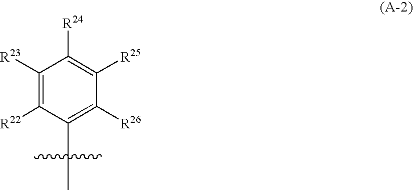
wherein:
one of R²³ and R²⁴ is R⁹, and the other of R²³ and R²⁴ is hydrogen, and each of R²², R²⁵, and R²⁶ is, independently, hydrogen or R^{e}.

10. The compound of claim 9, wherein R²³ is R⁹, and R²⁴ is hydrogen.

11. The compound of claim 9, wherein R²⁴ is R⁹, and R²³ is hydrogen.

12. The compound of claim 9, wherein each of R²², R²⁵, and R²⁶ is hydrogen.

13. The compound of claim 9, wherein one of R²², R²⁵, and R²⁶ is R^{e}, and the other two are hydrogen.

14. The compound of claim 13, wherein R²⁶ is R^{e}, and each of R²² and R²⁵ is hydrogen.

15. The compound of claim 14, wherein R²⁶ is halo.

16. The compound of claim 15, wherein R²⁶ is chloro.

17. The compound of claim 1, wherein W is —O—.

18. The compound of claim 1, wherein W is a bond.

19. The compound of claim 1, wherein A is C₆-C₁₀ aryl, which is (a) substituted with 1 R¹¹; and (b) optionally further substituted with from 1-4 R^{g}.

20. The compound of claim 1, wherein A is phenyl, which is (a) substituted with 1 R¹¹; and (b) optionally further substituted with from 1-4 R^{g}.

21. The compound of claim 20, wherein A has formula (B-1): 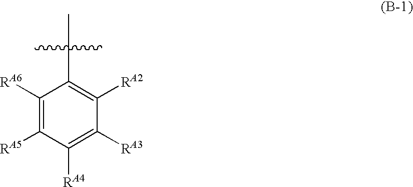
wherein:
one of R^{A3} and R^{A4} is R¹¹, the other of R^{A3} and R^{A4} is hydrogen; and
each of R^{A2}, R^{A5}, and R^{A6} is, independently, hydrogen or R^{g}.

22. The compound of claim 1, wherein R¹¹ is —W²—S(O)ₙR¹².

23. The compound of claim 22, wherein W² is a bond, and n is 2.

24. The compound of claim 22, wherein R¹² is C₁-C₆ alkyl, optionally substituted with from 1-2 R^{a}.

25. The compound of claim 24, wherein R¹² is CH₃.

26. The compound of claim 1, wherein R² has formula (A-2): 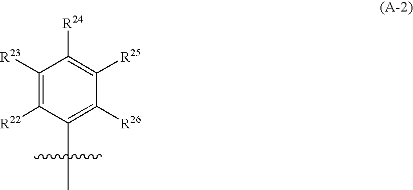
wherein:
one of R²³ and R²⁴ has formula (C-1): 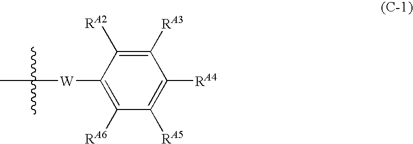
wherein one of R^{A2}, R^{A3}, R^{A4}, R^{A5}, and R^{A6} is R¹¹, and the others are each, independently, hydrogen or R^{g};
and the other of R²³ and R²⁴ is hydrogen; and
each of R²², R²⁵, and R²⁶ is, independently, hydrogen or R^{e}.

27. The compound of claim 26, wherein R²³ has formula (C-1), and R²⁴ is hydrogen.

28. The compound of claim 26, wherein each of R²², R²⁵, and R²⁶ is hydrogen.

29. The compound of claim 26, wherein one of R²², R²⁵, and R²⁶ is R^{e}, and the other two are hydrogen.

30. The compound of claim 29, wherein R²⁶ is R^{e}, and each of R²² and R²⁵ is hydrogen.

31. The compound of claim 30, wherein R²⁶ is halo.

32. The compound of claim 31, wherein R²⁶ is chloro.

33. The compound of claim 26, wherein W is —O—.

34. The compound of claim 26, wherein W is a bond.

35. The compound of claim 26, wherein one of R^{A3} and R^{A4} is R¹¹, and the other of R^{A3} and R^{A4} is hydrogen; and each of R^{A2}, R^{A5}, and R^{A6} is, independently, hydrogen or R^{g}.

36. The compound of claim 35, wherein R¹¹ is —W²—S(O)ₙR¹².

37. The compound of claim 35, wherein R^{A3} is R¹¹, and R^{A4} is hydrogen.

38. The compound of claim 37, wherein R¹¹ is —W²—S(O)ₙR¹².

39. The compound of claim 38, wherein W² is a bond, and n is 2.

40. The compound of claim 38, wherein R¹² is C₁-C₆ alkyl, optionally substituted with from 1-2 R^{a}.

41. The compound of claim 40, wherein R¹² is C₁-C₃ alkyl.

42. The compound of claim 41, wherein R¹² is CH₃.

43. The compound of claim 40, wherein R¹² is C₁-C₆ alkyl substituted with 1 R^{a}, wherein R^{a} is hydroxyl or NR^{m}Rⁿ.

44. The compound of claim 35, wherein each of R^{A2}, R^{A5}, and R^{A6} is hydrogen.

45. The compound of claim 35, wherein R^{A5} is R^{g}, and each of R^{A2} and R^{A6} is hydrogen.

46. The compound of claim 45, wherein R^{A5} is halo.

47. The compound of claim 37, wherein R¹¹ is —W²—S(O)ₙNR¹³R¹⁴.

48. The compound of claim 47, wherein W² is a bond, and one of R¹³ and R¹⁴ is C₁-C₃ alkyl, and the other of R¹³ and R¹⁴ is hydrogen.

49. The compound of claim 1, wherein one of R³ and R⁶ is:
(i) halo; or
(ii) C₁-C₆ alkyl or C₁-C₆ haloalkyl, each of which is optionally substituted with from 1-3 R^{a}; or
(iii) nitro; hydroxy; C₁-C₆ alkoxy; C₁-C₆ haloalkoxy; C₁-C₆ thioalkoxy; C₁-C₆ thiohaloalkoxy; cyano; or S(O)_{z}(C₁-C₃ alkyl), wherein z is 1 or 2;
and the other of R³ and R⁶ is:
(i) hydrogen; or
(ii) halo; or
(iii) C₁-C₆ alkyl or C₁-C₆ haloalkyl, each of which is optionally substituted with from 1-3 R^{a}; or
(iv) nitro; hydroxy; C₁-C₆ alkoxy; C₁-C₆ haloalkoxy; C₁-C₆ thioalkoxy; C₁-C₆ thiohaloalkoxy; cyano; or S(O)_{z}(C₁-C₃ alkyl), wherein z is 1 or 2.

50. The compound of claim 1, wherein one of R³ and R⁶ is:
(i) halo; or
(ii) C₁-C₆ alkyl or C₁-C₆ haloalkyl, each of which is optionally substituted with from 1-3 R^{a}; or
(iii) nitro; hydroxy; C₁-C₆ alkoxy; C₁-C₆ haloalkoxy; C₁-C₆ thioalkoxy; C₁-C₆ thiohaloalkoxy; cyano; or S(O)_{z}(C₁-C₃ alkyl), wherein z is 1 or 2;
and the other of R³ and R⁶ is hydrogen.

51. The compound of claim 1, wherein one of R³ and R⁶ is:
(i) halo; or
(ii) C₁-C₆ haloalkyl; or
(iii) C₁-C₆ alkoxy; C₁-C₆ haloalkoxy; or cyano;
and the other of R³ and R⁶ is hydrogen.

52. The compound of claim 1, wherein one of R³ and R⁶ is halo, the other of R³ and R⁶ is hydrogen.

53. The compound of claim 52, wherein one of R³ and R⁶ is chloro, the other of R³ and R⁶ is hydrogen.

54. The compound of claim 1, wherein one of R³ and R⁶ is C₁-C₄ haloalkyl, the other of R³ and R⁶ is hydrogen.

55. The compound of claim 54, wherein one of R³ and R⁶ is C₁-C₄ perfluoroalkyl, the other of R³ and R⁶ is hydrogen.

56. The compound of claim 55, wherein one of R³ and R⁶ is CF₃, the other of R³ and R⁶ is hydrogen.

57. The compound of claim 1, wherein each of R⁴ and R⁵ is hydrogen.

58. The compound of claim 1, wherein the compound has formula (VI): 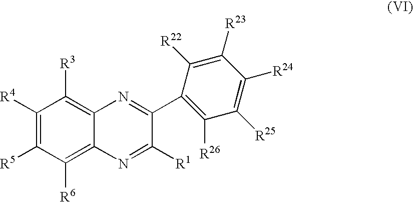
wherein:
R¹ is:
(i) hydrogen; or
(ii) C₁-C₃ alkyl or C₁-C₃ haloalkyl; or
(iii) NR⁷R⁸;
one of R³ and R⁶ is:
(i) halo; or
(ii) C₁-C₄ haloalkyl; or
(iii) C₁-C₆ alkoxy; C₁-C₆ haloalkoxy; or cyano;
and the other of R³ and R⁶ is hydrogen;
each of R⁴ and R⁵ is hydrogen;
one of R²³ and R²⁴ has formula (C-1): 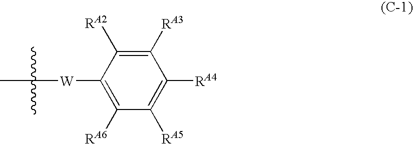
wherein one of R^{A2}, R^{A3}, R^{A4}, R^{A5}, and R^{A6} is R¹¹, and the others are each, independently, hydrogen or R^{g}; and W is a bond or —O—;
and the other of R²³ and R²⁴ is hydrogen, and
each of R²², R²⁵, and R²⁶ is, independently, hydrogen or R^{e}.

59. The compound of claim 58, wherein R¹ is CH₃.

60. The compound of claim 58, wherein R²³ has formula (C-1), and R²⁴ is hydrogen.

61. The compound of claim 58, wherein each of R²², R²⁵, and R²⁶ is hydrogen.

62. The compound of claim 58, wherein one of R²², R²⁵, and R²⁶ is R^{e}, and the other two are hydrogen.

63. The compound of claim 62, wherein R²⁶ is R^{e}, and each of R²² and R²⁵ is hydrogen.

64. The compound of claim 63, wherein R²⁶ is halo.

65. The compound of claim 64, wherein R²⁶ is chloro.

66. The compound of claim 58, wherein one of R^{A3} and R^{A4} is R¹¹, and the other of R^{A3} and R^{A4} is hydrogen; and each of R^{A2}, R^{A5}, and R^{A6} is, independently, hydrogen or R^{g}.

67. The compound of claim 66, wherein R^{A3} is R¹¹, and R^{A4} is hydrogen.

68. The compound of claim 67, wherein R¹¹ is —W²—S(O)ₙR¹².

69. The compound of claim 68, wherein W² is a bond, n is 2, and R¹² is C₁-C₆ alkyl, optionally substituted with from 1-2 R^{a}.

70. The compound of claim 69, wherein R¹² is CH₃.

71. The compound of claim 66, wherein each of R^{A2}, R^{A5}, and R^{A6} is hydrogen; or R^{A5} is R^{g}, and each of R^{A2} and R^{A6} is hydrogen.

72. The compound of claim 67, wherein R¹¹ is —W²—S(O)ₙNR¹³R¹⁴, wherein W² is a bond, and one of R¹³ and R¹⁴ is C₁-C₃ alkyl, and the other of R¹³ and R¹⁴ is hydrogen.

73. The compound of claim 58, wherein R³ is hydrogen, and R⁶ is CF₃.

74. The compound of claim 58, wherein R³ is CF₃, and R⁶ is hydrogen.

75. The compound of claim 1, wherein the compound is selected from the title compounds of Examples 1-42 and 44-83; or a pharmaceutically acceptable salt and/or N-oxide thereof.

76. A composition comprising a compound of claim 1 or a pharmaceutically acceptable salt thereof and a pharmaceutically acceptable carrier.

77. A method of preventing or treating a Liver X receptor-mediated disease or disorder, the method comprising administering to a subject in need of such treatment an effective amount of a compound of claim 1 or a pharmaceutically acceptable salt thereof.

78. A method of preventing or treating atherosclerosis, the method comprising administering to a subject in need of such treatment an effective amount of a compound of claim 1 or a pharmaceutically acceptable salt thereof.

79. A method of preventing or treating a cardiovascular disease, the method comprising administering to a subject in need of such treatment an effective amount of a compound of claim 1 or a pharmaceutically acceptable salt thereof.

80. The method of claim 79, wherein the cardiovascular disease is acute coronary syndrome or restenosis.

81. The method of claim 79, wherein the cardiovascular disease is coronary artery disease.

82. A method of preventing or treating Syndrome X, the method comprising administering to a subject in need of such treatment an effective amount of a compound of claim 1 or a pharmaceutically acceptable salt thereof.

83. A method of preventing or treating obesity, the method comprising administering to a subject in need of such treatment an effective amount of a compound of claim 1 or a pharmaceutically acceptable salt thereof.

84. A method of preventing or treating one or more lipid disorders selected from dyslipidemia, hyperlipidemia, hypertriglyceridemia, hypercholesterolemia, low HDL and/or high LDL, the method comprising administering to a subject in need of such treatment an effective amount of a compound of claim 1 or a pharmaceutically acceptable salt thereof.

85. A method of preventing or treating Alzheimer's disease, the method comprising administering to a subject in need of such treatment an effective amount of a compound of claim 1 or a pharmaceutically acceptable salt thereof.

86. A method of preventing or treating type I or type II diabetes, the method comprising administering to a subject in need of such treatment an effective amount of a compound of claim 1 or a pharmaceutically acceptable salt thereof.

87. A method of preventing or treating an inflammatory disease, the method comprising administering to a subject in need of such treatment an effective amount of a compound of claim 1 or a pharmaceutically acceptable salt thereof.

88. The method of claim 87, wherein the inflammatory disease is rheumatoid arthritis.

89. A method of treating a connective tissue disease, the method comprising administering to a mammal in need thereof an effective amount of a compound of claim 1 or a pharmaceutically acceptable salt thereof.

90. The method of claim 89, wherein the compound of formula (I) inhibits cartilage degradation and induces cartilage regeneration.

91. The method of claim 90, wherein the compound of formula (I) inhibits aggrecanase activity.

92. The method of claim 90, wherein the compound of formula (I) inhibits elaboration of pro-inflammatory cytokines in osteoarthritic lesions.

93. The method of claim 90, wherein the connective tissue disease is osteoarthritis or tendonitis.

94. The method of claim 90, wherein the mammal is a human.

95. A method of treating skin aging, the method comprising administering to a mammal in need thereof an effective amount of a compound claim 1 or a pharmaceutically acceptable salt thereof.

96. The method of claim 95, wherein the mammal is a human.

97. The method of claim 95, wherein the compound of formula (I) is topically administered.

98. The method of claim 95, wherein the skin aging is derived from chronological aging, photoaging, steroid-induced skin thinning, or a combination thereof.


SEQUENCE LISTING (3 sequences)

SEQ ID NO: 1 - DNA, 22 bp, Artificial
  Synthetic Forward Primer
caacatgaatgccattttccaa

SEQ ID NO: 2 - DNA, 21 bp, Artificial
  Synthetic Reverse Primer
ataatcccctgaacccaagga

SEQ ID NO: 3 - DNA, 25 bp, Artificial
  Synthetic Probe
taaagccatgccctctgcaggaaca